(19) United States
(12) Patent Application Publication
(10) Pub. No.: US 2013/0337463 A1
(43) Pub. Date: Dec. 19, 2013

(54) Cell-Based Models of Neurodegenerative Disease

(71) Applicants: Kurt R. Brunden, Media, PA (US); Virginia M.Y. Lee, Philadelphia, PA (US); John Q. Trojanowski, Philadelphia, PA (US); Kelvin C. Luk, Philadelphia, PA (US); Jing Guo, Philadelphia, PA (US); Laura Volpicelli-Daley, Birmingham, AL (US)

(72) Inventors: Kurt R. Brunden, Media, PA (US); Virginia M.Y. Lee, Philadelphia, PA (US); John Q. Trojanowski, Philadelphia, PA (US); Kelvin C. Luk, Philadelphia, PA (US); Jing Guo, Philadelphia, PA (US); Laura Volpicelli-Daley, Birmingham, AL (US)

(73) Assignee: The Trustees of the University of Pennsylvania, Philadelphia, PA (US)

(21) Appl. No.: 13/882,674

(22) PCT Filed: Nov. 4, 2011
(86) PCT No.: PCT/US2011/059360

§ 371 Date: Sep. 5, 2013

Related U.S. Application Data
(60) Provisional application No. 61/410,505, filed on Nov. 5, 2010.
(60) Provisional application No. 61/425,650, filed on Dec. 21, 2010.

(51) Int. Cl. G01N 33/68; G01N 33/50

(52) U.S. Cl. CPC G01N 33/6896; G01N 33/5058
USPC 435/6.13; 435/325; 435/29

(57) ABSTRACT

The present invention relates to a cell-based model useful for identifying molecules that modify intracellular pathways of α-synuclein and tau aggregation and degradation.

### BACKGROUND OF THE INVENTION

Alpha-synuclein (α-Syn) is a highly soluble natively unfolded protein expressed throughout the CNS. Although the close association of α-Syn with lipid membranes and enrichment at synaptic terminals suggest a role in synaptic maintenance and neurotransmitter release (Maroteaux et al., 1988, J. Neurosci. 8:2804-2815; Clayton et al., 1999, J Neurosci Res. 58:120-129), the precise physiological functions of α-Syn remain uncertain. Also, animals lacking the α-Syn gene (SNCA) show no obvious defects (Abeliovich et al., 2000, Neuron. 25:239-252). In contrast, intracellular accumulations comprised of highly organized α-Syn amyloid fibrils define a family of neurological disorders (the synucleinopathies) that includes Parkinson's disease (PD), dementia with Lewy bodies (LBs), multiple systems atrophy, and neurodegeneration with brain iron accumulation type 1 (Spillantini et al., 1997, Nature. 388:839-840).

Purified α-Syn readily assembles into amyloid-like fibrils similar to those in LBs under defined conditions in vitro and has been studied extensively (Conway et al., 1998, Nat. Med. 4:1318-1320; Uversly, 2007, J. Neurochem. 103:17-37). Fibrillization occurs through a two-step polymerization process, whereby soluble monomer is converted into conformationally distinct oligomeric intermediates, which then serve as nuclei for subsequent elongation (Wood et al., 1999, J Biol Chem, 274:19509-19512). Curiously, although α-Syn aggregation and pathology are prominent in humans and in animal models of synucleinopathies (Masliah et al., 2000, Science. 287:1265-1269; Lee et al., 2002, Proc Natl Acad Sci USA. 99:8968-8973; Giasson et al., 2002, Neuron. 34:521-533), overexpression of α-Syn in neuronal and normeuronal cells, as well as primary neurons derived from α-Syn transgenic mice, does not lead to significant α-Syn inclusion formation (Kahle et al., 2000, Ann N.Y. Acad Sci. 920:33-41). Indeed, this absence of cell models that recapitulate the morphological and biochemical features of LBs is a serious impediment to elucidating the pathological events or disease pathways leading to α-Syn aggregation in vivo.

Neurodegenerative tauopathies, including Alzheimer's disease (AD) and frontotemporal dementias (FTD), are characterized by neurofibrillary tangles (NFTs) composed of intracellular hyperphosphorylated tau aggregates (Kosik, et al., 1986, Proc. Natl. Acad. Sci. U.S.A. 83:4044-4048; Pollock, et al., 1986, Lancet. 2:1211; Goedert, et al., 1988, Proc. Natl. Acad. Sci. U.S.A. 85:4051-4055; Lee, et al., 1991, Science. 251:675-678). Predominantly expressed in neurons, tau is a microtubule (MT)-binding protein which stabilizes MTs and promotes their assembly (Witman, et al., 1976, Proc. Natl. Acad. Sci. U.S.A. 73:4070-4074; Drechsel, et al., 1992, Mol. Biol. Cell. 3:1141-1154), and tau-MT interactions are negatively regulated by phosphorylation of tau (Biernat, et al., 1993, Neuron. 11:153-163; Bramblett, et al., 1993, Neuron. 10:1089-1099). In adult brains, alternative splicing of the MAPT gene (Goedert, et al., 1989, Neuron. 3:519-526; Andreadis et al., 1992, Biochemistry. 31:10626-1063) generates 6 tau isoforms containing either 3 or 4 MT-binding repeats (3R or 4R tau) and 0-2 N-terminal inserts (0N, 1N or 2N tau).

A naturally unfolded soluble protein under normal conditions, tau acquires highly-ordered β-pleated sheet structures as it assembles into insoluble, hyperphosphorylated, 15-20 nm-diameter paired helical filaments as well as less frequent straight filaments that constitute NFTs in AD and related tauopathies (Kidd, M. 1963. Nature. 197:192-193, Berriman, et al., 2003, Proc. Natl. Acad. Sci. U.S.A. 100:9034-9038). Mechanisms underlying such dramatic conversions remain a conundrum. Significant correlations of total NFT burden with cognitive decline are observed in AD patients (Wilcock, et al., 1982, J. Neurol. Sci. 56:343-356; Arriagada et al., 1992, Neurology. 42:631-639) and, importantly, discoveries of over 30 dominantly inherited mutations in the MAPT gene in FTD with Parkinsonism linked to chromosome 17 (FTDP-17) (Hutton, et al., 1998, Nature. 393:702-705; Spillantini, et al., 1998, Am. J. Pathol. 153:1359-1363; Rizzu, et al., 1999, Am. J. Hum. Genet. 64:414-421; reviewed by Goedert, et al., 2005, Biochim. Biophys. Acta. 1739:240-250) strongly suggest a causal link between tau abnormality and neuronal dysfunction. Although the exact mechanisms of tau-mediated neurodegeneration are not well understood, both the loss of the MT-binding function of tau due to sequestration of soluble tau into tangles and toxic gains of function owing to the sheer physical occupancy of large intracellular aggregates have been proposed to explain the dire consequences of tau aggregation (reviewed by Lee, et al., 1994 Neurobiol. Aging. 15 Suppl 2:S87-9; Ballatore, et al., 2007, Nat. Rev. Neurosci. 8:663-672).

The common involvement of α-synuclein in a spectrum of diseases such as Parkinson's disease, dementia with Lewy bodies, multiple system atrophy and the Lewy body variant of Alzheimer's disease has led to the classification of these diseases under the umbrella term of “synucleinopathies.” Protecting neurons from the toxic effects of alpha-synuclein is a promising strategy for treating these diseases. Similarly, fibrillar aggregates of tau protein are found as NFTs in Alzheimer's disease and a number of related “tauopathies”, including Pick's disease, supranuclear palsy and certain Frontotemporal lobar degenerative diseases. There is thus a need in the art for systems that permit the studying of α-Syn and tau protein in the development of diseases. The present invention addresses this unmet need in the art.

### SUMMARY OF THE INVENTION

The invention provides a cell culture model for alpha synuclein inclusion formation. In one embodiment, the model comprises a cell population and an exogenous alpha synuclein fibril.

In one embodiment, the cell population comprises a neuronal cell. In another embodiment, the cell population comprises a non-genetically modified cell.

In yet another embodiment, the cell population comprises a cell engineered to express a nucleic acid encoding alpha synuclein. In one embodiment, the cell further comprises a nucleic acid encoding a detectable protein.

In one embodiment, the cell culture model comprises a culture medium comprising an exogenous alpha synuclein fibril.

In one embodiment, the cell culture model comprises wheat germ agglutinin (WGA).

In one embodiment, the exogenous alpha synuclein fibril is derived from a mammalian alpha synuclein. In one embodiment, the mammalian alpha synuclein is selected from the group consisting of mouse, rat, primate, and human.

In one embodiment, the exogenous alpha synuclein fibril is derived from a human alpha synuclein or a fragment thereof selected from the group consisting of full-length alpha synuclein (α-syn), α-syn-1-120, α-syn-1-89, α-syn-58-140, α-syn-61-95, and any combination thereof.

The invention also provides a cell culture model for tau inclusion formation. In one embodiment, the model comprises a cell engineered to express a nucleic acid encoding tau.

In one embodiment, the tau has a P301L mutation.

In one embodiment, the cell is a neuronal cell. In some instances, the cell further comprises a nucleic acid encoding a detectable protein.

In one embodiment, the cell culture model comprises a culture medium comprising exogenous tau.

The invention also provides a method of identifying a test agent that inhibits filament aggregation. In one embodiment, the method comprises contacting a cell culture model for filament aggregation with the test agent and comparing the amount of aggregation in a cell in the cell culture model with the amount of aggregation in a cell in an otherwise identical cell culture model not contacted with the test agent, wherein a lower level of aggregation in the presence of the test agent identifies the test agent as an inhibitor of filament aggregation.

In one embodiment, the cell culture model is a model for alpha synuclein inclusion formation, wherein the model comprises a cell population and an exogenous alpha synuclein fibril. In another embodiment, the cell culture model is a model for tau inclusion formation comprising a cell engineered to express a nucleic acid encoding tau.

The invention also provides a method of identifying a gene product that modulates filament aggregation in a cell. In one embodiment, the method comprises contacting a cell culture model for filament aggregation with a modulator of gene expression and comparing the amount of aggregation in a cell in the cell culture model with the amount of aggregation in a cell in an otherwise identical cell culture model not contacted with the modulator of gene expression, wherein a change in the level of aggregation in the presence of the modulator of gene expression identifies the modulator of gene expression as a gene product that modulates filament aggregation.

In one embodiment, the cell culture model is a model for alpha synuclein inclusion formation, wherein the model comprises a cell population and an exogenous alpha synuclein fibril. In another embodiment, the cell culture model is a model for tau inclusion formation comprising a cell engineered to express a nucleic acid encoding tau.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A through 1O, is a series of images depicting intracellular fibrils seeding α-Syn aggregation. (A-F) Monomeric fluorescently labeled α-Syn (α-Syn⁵⁹⁴) or α-Syn⁵⁹⁴ PFFs were delivered into QBI-WT-Syn cells by using a cationic-liposome reagent (Bioporter). Cells were passaged after 4 h and visualized under DIC and fluorescence-microscopy after fixation at 24 h. Both transduced α-Syn⁵⁹⁴ monomer (A-C, arrowheads) and pre-formed fibrils (PFF; D-F, arrows) could readily be detected within cell boundaries (dashed lines) indicating efficient intracellular delivery. (G-L) QBI cells stably expressing A53T-Syn were transduced with WT-α-Syn PFFs, fixed at 48 h, and immunostained with either a pan-α-Syn antibody (SNL4; G) or a monoclonal antibody specifically recognizing misfolded α-Syn (Syn506; J). Co-labeling with Alexa Fluor 488-conjugated PHA-L (PHA) was used to reveal the plasma membrane. Confocal microscopy shows large α-Syn-positive intracellular inclusions in fibril-seeded cells (G-I and J-L). (M-O) A 3D view of an inclusion-bearing cell reconstructed from serial confocal images. Removal of the PHA signal (green) reveals the juxtanuclear position of a single prominent α-Syn aggregate and confirmed its intracellular location. (P) Quantification of α-Syn inclusions in QBI-WT-Syn cells seeded with either WT-Syn monomer or PFFs (data from three separate transductions from two independent experiments; n>500 cells per condition). [Scale bars, 10 μm (F); 6 μm (G); 15 μm (j).]

FIG. 2, comprising FIGS. 2A through 2P, is a series of images depicting the resemblance between seeded inclusions and human LBs. LBs in the cingulate cortex of a PD patient with dementia showing strong immunoreactivity for α-Syn (A), phosphorylated α-Syn (pSyn) (E), ubiquitin (Ubi) (I), and positive staining with the amyloid-specific dye ThS (M). Intracellular inclusions formed by seeding with recombinant WT-α-Syn fibrils also stained positively with a monoclonal antibody specific to misfolded α-Syn (Syn 506; B-D). Inclusions were also strongly phosphorylated (F-H) and ubiquitinated (J-L), and detectable by using additional α-Syn antibodies SNL1 (B), SNL4 (G) and Syn303 (K). Confocal images demonstrating intense ThS labeling in the core region of inclusions, in contrast to phosphorylation, which was strongest in the periphery (N-P). Cell boundary is indicated in white. [Scale bars, 10 μm (A, E, I, and M); 10 μm (D, H, and L); 5 μm (P).]

FIG. 3, comprising FIGS. 3A through 3K, is a series of images demonstrating that soluble endogenous Syn is recruited into inclusions by fibrils. (A-C) QBI-WT-Syn cells were seeded with fibrils generated by using recombinant α-Syn containing a C-terminal Myc-tag. Double immunostaining for Myc and anti-α-Syn^{pSer129} (pSyn) revealed that fibril seeds form the core of inclusions whereas pSyn predominates in the periphery regions. (D) Immunoblot of detergent (Triton X-100)-soluble (TriX) and detergent-insoluble (SDS) fractions of cell lysates from control unseeded QBI-WT-Syn cells. (E and F) Lysates from cells transduced with Syn-Myc fibrils contained both WT (black arrowhead) and Syn-Myc (white arrowhead) in the TriX-insoluble, SDS-soluble fraction, indicating that α-Syn originating from the cell comprise the majority of α-Syn within inclusions. A smear representing high molecular weight α-Syn species (**) could also be detected in the SDS fraction. (G,H) Remaining Syn-Myc seeds within the SDS-fraction of transduced lysates were immunoprecipitated with anti-Myc (9E10). (G) Antibodies against α-Syn (SNL-4) indicate efficient pull-down of Syn-Myc seeds. (H) Probing with anti-pSyn indicates that phosphorylation occurs overwhelmingly in endogenous α-Syn but not exogenous fibrils *, IgG light chain; FT, flow-through fraction. (I and J) Inclusions detected in cells stably expressing Myc-tagged α-Syn (QBI-Syn-Myc) transduced with WT α-Syn PFFs. Positive staining for Myc (I) and Syn303 (J) indicates that inclusions contain α-Syn of cellular origin. [Scale bars, 2.5 μm (A); 5 μm (I)]

FIG. 4, comprising FIG. 4A through FIG. 4I, is a series of images demonstrating that inclusion formation does not require the α-Syn N- or C-terminal regions in seeds. (A-D) QBI-A53T-Syn cells were transduced with α-Syn PFFs lacking either the N-terminal (α-Syn²¹⁻¹⁴⁰) or C-terminal domain (α-Syn¹⁻¹²⁰). Transduced cells were immunostained using antibodies recognizing α-Syn at either the extreme N-terminal (Syn303; A) or C-terminal (Syn211; C); thus, detecting only endogenously produced α-Syn. Both truncated forms of fibrils recruited cellular α-Syn as indicated by inclusion formation. Inclusions were phosphorylated (pSyn; B,D), indicating that their formation does not depend on seed phosphorylation or interaction with membranes. (E and F) Lysates from cells transduced with either WT-α-Syn monomer (Mono), full-length (Syn PFF), or truncated α-Syn fibrils were probed with antibodies against α-Syn (SNL4) and α-Syn^{pser129}. Immunoblot with SNL-4 (E) shows full-length endogenous α-Syn within Triton-insoluble (SDS-soluble) fractions of cells transduced with WT, α-Syn²¹⁻¹⁴⁰, and α-Syn¹⁻¹²⁰ PFFs, indicating that cellular α-Syn is converted by fibril seeds. Transduction also resulted in the appearance of high molecular weight α-Syn species (*) consistent with ubiquitination. Fibril transduction also led to a dramatic increase in the amount of phosphorylated α-Syn found almost exclusively within SDS-soluble fractions (F Upper) consistent with its location within insoluble inclusions. GAPDH loading controls are also shown (F Lower). (G) QBI-Δ71-82-Syn cells transduced with WT fibrils did not form inclusions, indicating that the core fibril assembly region of α-Syn is critical to recruitment and incorporation. (H and I) QBI-cells stably expressing α-Syn mutated at Ser-129 (S129A) were transduced with PFFs prepared from α-Syn-S129A (H) or α-Syn¹⁻¹²⁰ (I), which lack this phosphorylation site. Double immunostaining with Syn506 and SNL4 indicate the formation of misfolded α-Syn inclusions. [Scale bars, 5 μm (A-D); 5 μm (G-I).]

FIG. 5, comprising FIGS. 5A through 5N, is a series of images demonstrating that fibril-seeded α-Syn inclusions are associated with vesicular bodies. (A-C) EM images of cells stably expressing A53T-α-Syn 24 h after transduction with either WT-α-Syn monomer (A) or PFFs (B and C). Electron dense inclusions (in) were found only in the cytoplasm of PFF-seeded cells. Nucleus (Nu) and cytoplasm (Cyt) are also indicated. (C) High-power magnification of the region delineated in B revealing a fibrillar core (black arrows and Inset) consistent with fibrils serving as a nidus for recruiting endogeous α-Syn. Multilamellar bodies are also present in the surrounding cytoplasm (arrowhead). (D and E) Immuno-EM using anti-Myc (9E10) in QBI-Syn-A53T cells transduced with Syn-Myc PFFs. Exogenous (Myc-tagged) fibrils are localized to the perinuclear region 6 h after treatment (D). At 16 h, vesicular organelles, likely containing α-Syn, are recruited to PFF seeds (E and F). [Scale bars, 2 μm (A, B, and E); 400 nm (C); 0.5 μm (D and F).]Double-immunostaining against pSyn and Hsp70 (G-I) reveal molecular chaperones colocalized to inclusions 48 h after transduction with Syn-Myc PFFs. Whereas pSyn is found at the periphery, Hsp70 is detected throughout inclusions, suggesting it is also recruited to PFF seeds. (J-L) Antibodies against α-Syn^{pSer129} (J) and GM130 (K) were used to label inclusions and the Golgi matrix, respectively. Cells lacking aggregates display compact Golgi morphology (asterisks), whereas inclusion-bearing cells show fragmented GM130 staining (arrows). (M and N) Golgi dispersal was assessed by measuring the area stained by GM130 (M) and average pixel intensity (N) in Myc-Syn PFF-transduced cells with or without phosphorylated α-Syn inclusions. The majority of inclusion-bearing cells displayed fragmentation as reflected by an increase in area positive for GM130 with a concomitant decrease in staining intensity. *, P<0.001; #, P<0.005 t test (results obtained from three separate transduction experiments, n=200).

FIG. 6, comprising FIGS. 6A through 6K, is a series of images depicting the ultrastructure of α-Syn fibrils and their transduction into cultured cells. (a-e) Fibrils assembled from full-length WT, C-terminal truncated (1-120), N-terminal truncated (21-140), or C-terminal Myc-tagged (SynMyc). Oligomers formed for dopamine-treated α-Syn were also prepared (e). Preparations were briefly sonicated and visualized by transmission EM after negative staining with 1% uranyl acetate. Electron micrographs represent preparations used in transduction experiments. (Scale bar, 100 nm.) (f-k) QBI-WT-Syn cells were transduced by using Bioporter with monomeric α-Syn594 (f-h) or α-Syn594 PFFs (i-k) and passaged onto PDL-coated glass coverslips after 4 h. Cells were fixed and visualized by fluorescence microscopy for Alexa Fluor 594 signal 24 h after transduction. Bright punctate staining was observed dispersed within the cytosol of monomer-transduced cells, whereas PFF-transduced cells contained larger amorphous aggregates. [Scale bars, 10 μm (h and k).]

FIG. 7, comprising FIGS. 7A through 7L, is a series of images depicting transduction of fibrillar and nonfibrillar α-Syn into WT-Syn cells. (a-i) QBI-WT-Syn cells transduced with either fibrillar WT-Syn or various forms of soluble α-Syn. After fixation 48 h after transduction, cells immunostained by using Syn506, which recognizes misfolded α-Syn, and a pan-α-Syn antibody (SNL4). (a-c) Fibril transduction led to the appearance of large intracellular α-Syn inclusions that contained misfolded α-Syn (arrows and Inset). In contrast, transduction with either recombinant α-Syn monomer (d-f), assembly-incompetent α-Syn Δ71-82 (g-i), or purified α-Syn oligomers (f-l) generated by exposure to dopamine did not lead to inclusion formation as detected by the two antibodies, indicating that fibrillar α-Syn alone can act as seeds for recruiting and converting α-Syn into intracellular inclusions. [Scale bars: 15 μm (c, f, i, and l).]

FIG. 8, comprising FIGS. 8A through 8G, is a series of images depicting intracellular α-Syn inclusions with multiple morphologies. (a-f) Inclusions in QBI-WT-Syn cells transduced with WT-Syn PFFs immunostained with anti-α-Syn-pSer129 (pSyn) and SNL4 revealing two main types of morphologies. Dense circular inclusions (a-c) stained strongly for α-Syn (as marked by SNL4) and were surrounded by a halo of pSyn. A second filamentous pattern of staining (d-f) was also observed, most frequently at proximal time points, likely representing inclusions at earlier stages of formation. Selected confocal slices of a filamentous inclusion stained against phosphorylated α-Syn (g) showing the elaborate network-like organization of these structures, which extends through the cytoplasm, but are not observed in the nucleus. [Scale bars, 10 μm (c, f and g).]

FIG. 9, comprising FIGS. 9A through 9F, is a series of images depicting the ability of preformed α-Syn fibrils to seed inclusion formation in multiple cell types. WT α-Syn or Syn-MycPFFs were transduced by using liposomes into HeLa cells transiently transfected with pcDNA3.1/WT-Syn or SH-SY5Y neuroblastoma cells stably expressing full-length human α-Syn. Round perinuclear α-Syn inclusions similar to those found in QBI-293 cells could be detected in both HeLa (a-c) and SH-SY5Y (d-f) cells. Inclusions in both cell types also contained phosphorylated α-Syn (b,e). [Scale bars, 20 μm (c); 10 μm (f).]

FIG. 10, comprising FIGS. 10A through 10G, is a series of images depicting the detergent solubility of phosphorylated intracellular α-Syn inclusions and preformed fibril preparations. QBI-WT-Syn cells transduced with Syn-Myc PFFs were fixed with 4% paraformaldehyde and extracted with 1% Triton X-100. Soluble cytoplasmic α-Syn was removed with extraction as shown by α-Syn (SNL4) staining. In contrast, phosphorylated inclusions with both filamentous (a and b) and dense (c and d) morphologies remained. Note that dense inclusions retain a halo pattern of staining with the α-Syn-pSer129 antibody, indicating that endogenous α-Syn within inclusions is also insoluble. (e and f) Dense inclusions stained with Syn506, demonstrating that α-Syn with abnormal conformation is distributed throughout insoluble inclusions. (g) Solubility was assessed in PFFs assembled from full-length WT, C-terminal truncated (1-120), N-terminal truncated (21-140), C-terminal Myc-tagged (Myc) α-Syn, or a Syn where residue 129 was mutated to alanine (S129A). Fibrils were sonicated in buffer containing 1% Triton-X followed by centrifugation at 100,000 μg for 30 min. Insoluble material was washed and resuspended in 1% SDS buffer. Triton-soluble (T) and SDS-soluble (S) fractions were separated by SDS/PAGE and probed by using SNL-4.

FIG. 11, comprising FIGS. 11A through 11L, is a series of images depicting the characterization of intracellular α-Syn inclusions. QBI-WT-Syn cells transduced with Syn-Myc PFFs were double-immunostained by using antibodies against intracellular markers. Phosphorylated α-Syn inclusions were colocalized to heat shock protein 90 (Hsp90; a-c), but not with cytoskeletal components such as microtubules (α-tubulin; d-f), actin filaments (g-i), or the intermediate filament vimentin (j-l). [Scale bars, 10 μm (c, i, and l); 20 μm (f).]

FIG. 12, comprising FIGS. 12A through 12X, is a series of images demonstrating the lack of efficient transmission of fibril-seeded inclusions between QBI-WT-Syn cells. To determine whether α-Syn inclusions formed by intracellular seeding with fibrils in this current model can be transferred to nonseeded cells, separate populations of fibril-transduced (donor) and nontransduced (recipient) cells were coincubated together. (a-c) Recipient cells (QBI-WT-Syn) were labeled by transfection with GFP and do not form inclusions as confirmed by lack of phosphorylated α-Syn (pSyn) staining (b). (d-f) Untransfected QBI-WT-Syn cells transduced with Syn-Myc fibrils form robust phosphorylated inclusions by 72 h post-delivery (e) and were used as donors. (g-o) Donor and recipient cells were cocultured at a 1:1 ratio and fixed at the indicated time points. Despite the abundance of inclusions, none colocalized with GFP, suggesting that inclusions were not transferred to recipient cells via coincubation. (p-u) High-power images showing examples of recipient cells (green) in direct contact with inclusion-bearing donor cells (white arrows). Inclusions (red) were found strictly within non-GFPcells, indicating that direct cell-cell contact does not result in transmission of α-Syn inclusions. (v-x) QBI-WT-Syn cells transfected with GFP followed by transduction with Syn-Myc fibrils were used as a positive control. Inclusions containing pSyn can be clearly detected within fluorescent cell bodies, indicating that recipient cells are capable of forming inclusions when fibrils are delivered intracellularly. [Scale bar, 5 μm (r).]

FIG. 13 is a schematic of cell-based model of tau inclusion formation.

FIG. 14, comprising FIGS. 14A through 14O, is a series of images depicting tau PFFs-seeded fibrillization in tau expressing WBI293 cells. (a-f) The intracellular fibrillization of tau can be seeded by both pre-formed myc-T40 fibrils (a-c) and mycK18 fibrils (d-f): green is monoclonal phopho-tau antibody PHF-1; red is polyclonal myc antibody. (g-i) The induced hyperphosphorylated tangle-like structures can be recognized by PHF-1 (green) and polyclonal tau antibody 17025 (red). (j-l) Intracellular tau aggregates show different morphologies (j:fibrillar; k:compact; l:mixed) revealed by MC-1 antibody (green) that recognizes tau in a pathological conformation. (m-o) Phosphorylated tau aggregates are occasionally ubiquitinated: green is PHF-1 antibody; red is ubiquitin antibody.

FIG. 15 is an image depicting tau PFF-seeded PS19 hippocampal neurons showing profound accumulation of phosphorylated and insoluble tau. The antibodies that were used to visualize insoluble tau aggregates that remain after fixation, permeabilization and extraction with 1% Triton X-100 are indicated in the figure.

FIG. 16, comprising FIGS. 16A through 16C, is a series of images demonstrating that Tau PFFs seed endogenous tau fibrillization in wtT40-transfected QBI-293 Cells. FIG. 16A is an image demonstrating that phosphorylated tau recognized by phospho-tau antibody PHF-1 was completely soluble in wtT40-transfected cells treated with BioPORTER reagent alone. Soluble phosphorylated tau wasextracted by 1% Triton-X100 during fixing in (b) but not in the absence of detergent (a). FIG. 16B is an image demonstrating that intracellular accumulation of insoluble tau recognized by PHF-1 can be induced by both myc-T40 and myc-K18 PFFs transduced using BioPORTER reagent. Exogenous PFFs were immunostained with polyclonal anti-myc antibody (red), and phosphorylated tau was immunstained with PHF-1 antibody (green). FIG. 16C is an image demonstrating that induced tau aggregates show a range of morphologies which can be recognized by the phospho-tau antibody AT8 and the conformational-dependent tau antibody MC-1. In FIGS. 16B and 16C, cells were extracted with 1% Triton-X100 during fixation. In all panels, cell nuclei were stained by DAPI (blue). Magnification: 20× for (FIG. 16A); 40× for (FIG. 16B) and (FIG. 16C). Scale bars: 100 μm for (FIG. 16A); 10 μm for (FIG. 16B) and (FIG. 16C).

FIG. 17, comprising FIGS. 17A through 17C, is a series of images demonstrating that the P301L mutation enhances PFF-induced tau aggregation. FIG. 17A is an image depicting QBI-293 cells transiently transfected with T40 harboring the P301L mutation (T40/P301L) and transduced with myc-K18 fibrils (a) or myc-K18/P301L fibrils (b) using BioPORTER reagent. Abundant tau aggregates were detected using mAb PHF-1 (green) following 1% Triton-X100 extraction during fixation to remove soluble proteins. FIG. 17B is an image illustrating protein distribution after sequential extraction was performed on T40/P301L-transfected cells treated with Bioporter reagent alone (control), or with myc-K18 fibrils (K18 fib) or myc-K18/P301L fibrils (K18/PL fib). T: cellular fraction recovered in 1% Triton-X100 lysis buffer. S: Triton-insoluble fraction solubilized in 1% SDS lysis buffer. Equal proportions of Triton and SDS fractions were loaded on SDS-PAGE gels. Immunobloting with polyclonal tau Ab 17025 and PHF-1 revealed accumulation of Triton-insoluble tau in fibril-transduced cells. GAPDH served as a loading control. FIG. 17C is an image depicting that a sandwich tau ELISA on cell lysates from 4 independent experiments confirmed induction of significant amount of Triton-insoluble tau after fibril transduction which was always accompanied by a reduction in soluble tau. Magnification: 40×. Scale bar: 50 μm.

FIG. 18, comprising FIGS. 18A and 18B, is a series of images demonstrating that internalization of small quantities of tau PFFs is sufficient to induce robust tau aggregation. QBI-293 cells were transiently transfected with mutant T40/P301L tau and transduced with myc-K18/P301L PFFs using BioPORTER reagent. FIG. 18A is an image demonstrating that incubation of fibril-transduced cells with polyclonal anti-myc antibody before fixing (live, red) followed by staining of fixed and permeabilized cells with anti-myc mAb 9E10 (fixed, green) showed extensive colocalization of the two antibodies, suggesting the majority of the PFFs detected were extracellular and associated with cell membranes. Arrows point to internalized fibrils which were only recognized by 9E10 antibody applied to fixed and permeabilized cells but not polyclonal anti-myc antibody used on live cells. FIG. 18B is an image demonstrating that double-labeling of phospho-aggregates by PHF-1 and exogenous PFFs by polyclonal anti-myc showed very little colocalization of the antibodies. Arrows point to aggregates with colocalizing PFF seeds. Magnification: 20×. Scale bar: 100 μm.

FIG. 19, comprising FIGS. 19A and 19B, is a series of images demonstrating that PFF-induced tau aggregates resemble neurofibrillary tangles in tauopathies. T40/P301L-transfected QBI-293 cells were transduced with myc-K18/P301L fibrils using BioPORTER reagent. FIG. 19A is an image depicting immunocytochemical staining with PHF-1 showing dense aggregates. FIG. 19B are images depicting immuno-EM of PFF-transduced cells using PHF-1 antibody to show abundant filamentous tau aggregates in the cytoplasm. Magnification: 20× for (FIG. 19A); 12,000× for (FIG. 19B) (a); 30,000× for (FIG. 19B) (b). Scale bars: 100 μm for (FIG. 19A); 2 μm for (FIG. 19B) (a); 500 nm for (FIG. 19B) (b).

FIG. 20, comprising FIGS. 20A through 20C, is a series of images depicting time-dependent development of insoluble tau aggregates. QBI-293 cells transfected with T40/P301L mutant tau were transduced with myc-K18/P301L PFFs using BioPORTER reagent. Soluble proteins were extracted by 1% Triton-X100 during fixation. FIG. 20A is an image demonstrating that at t=3 hr after the addition of PFF/reagent complex, a small percentage of cells started showing accumulations of insoluble tau (PHF-1) with focal inclusions co-localizing with PFF staining (myc). FIG. 20B is an image demonstrating that at t=6 hr, more cells developed aggregates, most of which are skein-like and diffusely distributed throughout the cytoplasm. A small population of cells showed large ThS-positive aggregates. FIG. 20C is an image demonstrating that large aggregates recognized by ThS were more frequently seen at t=24 hr after fibril addition. Magnification: 40×. Scale bar: 50 μm.

FIG. 21, comprising FIGS. 21A and 21B, is a series of images demonstrating that newly synthesized tau is rapidly recruited into insoluble aggregates. One day after myc-K18/P301L PFF transduction of QBI-293 cells transiently transfected with T40/P301L, cells were pulsed with [³⁵S]-methionine for 20 min and chased for different durations (0 to 6 hr). Cell lysates were sequentially extracted using 1% Triton-X100 followed by 1% SDS lysis buffer, and tau was immunoprecipitated from both fractions with the mAbs T46 and Tau 5. Equal proportions of Triton (T) and SDS (S) fractions were separated on SDS-PAGE for autoradiography. FIG. 21A is an autoradiograph indicating that newly synthesized tau remained soluble in cells treated with reagent alone (Rg control) but rapidly turned insoluble in fibril-transduced cells (Fib Td). FIG. 21B is an image depicting quantification from 2 independent experiments showing the distribution of radiolabeld tau in the soluble and insoluble fractions over time (Error bar: standard error of the mean).

FIG. 22, comprising FIGS. 22A through 22F, is a series of images demonstrating that Tau aggregation results in reduced MT stability. FIG. 22A is an image demonstrating that QBI-293 cells transfected with T40/P301L and transduced with Myc-K18 (K18 fib) and myc-K18/P301L (K18/PL fib) PFFs showed a significant reduction in MT-bundling compared to control cells treated with BioPORTER transduction reagent alone (control), as demonstrated by acetylated-tubulin (Ac-tub) staining. FIG. 22B is an image demonstrating by double-staining with PHF-1 and Ac-tub that cells with MT bundles exhibited diffuse tau immunostaining (arrows), whereas those with tau aggregates often lacked bundling (arrow heads). FIG. 22C is an image depicting quantification from 3 independent experiments which demonstrated statistically significant reduction in the percentage of cells with MT bundles in the presence of fibril transduction (Error bar: standard error; *: p<0.05). FIG. 22D is an image demonstrating a significant decrease in Ac-tub levels associated with fibril transduction using an Ac-tub sandwich ELISA performed on samples from 4 independent experiments (Error bar: standard error; *: p<0.05). FIGS. 22E and 22F are images demonstrating that the Ac-tub and tau sandwich ELISAs revealed a highly significant correlation of Ac-tub levels with Triton-soluble tau (E: p<0.0005), but not with Triton-insoluble tau (F: p>0.05), across control and fibril-transduced samples from 4 independent experiments. Magnification: 20× for (FIG. 22A); 40× for (FIG. 22B). Scale bar: 100 μm for (FIG. 22A); 50 μm for (FIG. 22B).

FIG. 23, comprising FIGS. 23A through 23F, is a series of images demonstrating that spontaneous uptake of PFFs without transduction reagent also induces intracellular tau aggregation. FIG. 23A is an image depicting T40/P301L aggregation induced by myc-K18/P301L PFFs alone in the absence of BioPORTER after 30 hr incubation at 37° C. (a) or 4 hr incubation at 37° C. (b). Minimal aggregation was observed with 4 hr incubation at 4° C. (c). For all conditions, cells were trypsinized and replated after the designated incubation period and fixed 48 hr after the initial addition of fibrils. Soluble proteins were extracted with 1% Triton-X100 during fixing. FIG. 23B is an image demonstrating that 48 hr incubation with myc-K18/P301L PFFs at 37° C. led to significant accumulation of Triton-insoluble T40/P301L detected on immunoblots. Control cells were transfected with T40/P301L without fibril transduction. FIG. 23C is an image demonstrating that 4 hr incubation with myc-K18/P301L fibrils at 37° C. but not at 4° C. resulted in detectable T40/P301L Triton-insoluble aggregates. FIG. 23D is an image demonstrating that quantification from 4 independent experiments showed significant reduction in aggregate-bearing cells when a 4 hr PFF incubation was performed at 4° C. instead of 37° C. (Error bar: standard error of the mean; *: p<0.05 compared to 4 hr incubation at 37° C.). Thirty-hour incubation with tau PFFs at 37° C. resulted in the highest incidence of aggregates (n=3; Error bar: standard error of the mean; **: p<0.01 compared to 4 hr incubation at 37° C.). FIG. 23E is an image demonstrating that Myc-tagged PFFs were barely visible by immunofluorescence in cells transduced with PFFs alone without BioPORTER reagent. FIG. 23F is an image demonstrating that exogenous myc-tagged tau PFFs were non-detectable with anti-myc mAb 9E10 via immunobloting under various transduction paradigms: transduction of PFFs with BioPORTER reagent (fib Td with Rg), 30 hr incubation with PFFs alone (30 hr fib alone), or 4 hr incubation with PFFs alone (4 hr fib alone). Cells were trypsinized and replated to new wells after the designated incubation time so that the majority of extracellular-associated PFFs were removed. Total fib input: the amount of tau PFFs used for transduction was added to cells immediately before lysing. *: non-specific bands. For FIGS. 23B, 23C and 23F, T: 1% Triton-X100 fraction; S: 1% SDS fraction. Equal proportions of Triton and SDS fractions were loaded on SDS-PAGE gels. Magnification: 20×. Scale bar: 100 μm.

FIG. 24, comprising FIGS. 24A through 24G, is a series of images demonstrating that wheat germ agglutinin (WGA) promotes PFF-induced tau aggregation by enhancing spontaneous PFF uptake. FIG. 24A is an image depicting T40/P301L-transfected cells that were incubated with myc-K18/P301L PFFs in the presence of 0, 5, 10 and 15 g/ml of WGA (0, 5, 10, 15) without BioPORTER reagent. Increasing doses of WGA resulted in more frequent phospho-tau aggregates, as visualized with PHF-1 antibody when 1% Triton-X100 was added during fixation. FIG. 24B is an image demonstrating that quantification from 3 independent experiments indicates that WGA dose-dependently increased the percentage of cells with aggregation (Error bar: standard error; *: p<0.05; **: p<0.01). FIG. 24C is an image demonstrating that WGA treatment increased accumulation of Triton-insoluble tau as shown on immunoblots. T: 1% Triton-X fraction; S: 1% SDS fraction. Equal proportions of Triton and SDS fractions were loaded on SDS-PAGE. FIG. 24D is an image demonstrating that WGA (0, 5, 10 and 15 g/ml) also increased the amount of cell-associated PFFs in a dose-dependent manner as revealed by myc immunostaining. FIG. 24E is an image demonstrating that two-stage immunostaining performed on cells treated with 15 μg/ml of WGA showed frequent occurrence of internalized PFFs that were only labeled by 9E10 that was applied to fixed and permeabilized cells, but not polyclonal anti-myc antibody that was incubated with live cells. Arrows point to examples of truly intracellular PFFs. FIG. 24F is an image demonstrating that N-acetyl glucosamine (GlcNAc), which inhibits the binding of WGA to plasma membrane, dramatically attenuated the aggregation-enhancing effects of WGA. FIG. 24G is an image demonstrating that quantification of results of 3 independent experiments showed significant reduction of tau aggregation in WGA-treated fibril-transduced cells in the presence of GlcNAc (Error bar: standard error of the mean; *: p<0.05). Magnification: 20× in (FIG. 24A) and (FIG. 24F); 40× in (FIG. 24D) and (FIG. 24E). Scale bars: 100 μm in (FIG. 24A) and (FIG. 24F); 50 μm in (FIG. 24D) and (FIG. 24E).

FIG. 25, comprising FIGS. 25A through 25F, is a series of images depicting transmission electron micrographs of negatively-stained PFFs composed of recombinant myc-T40 (FIGS. 25A and 25B), myc-K18 (FIGS. 25C and 25D), or myc-K18/P301L (FIGS. 25E and 25F). FIGS. 25A, 25C and 25D show fibril preparations prior to sonication; FIGS. 25B, 25D and 25F show sonicated fibrils used for transduction experiments. Magnification: 100,000×. Scale bar: 200 nm.

FIG. 26A is an image depicting Triton-insoluble tau aggregates after PHF-1 immunostaining in wtT40-transfected cells with myc-K18 (a) or myc-K18/P301L (b) PFF transduction. Soluble proteins were removed by 1% Triton-X100 extraction during fixing. FIG. 26B is an image demonstrating that a small amount of Triton-insoluble tau could be detected in a western blot with both myc-K18 and myc-K18/P301L PFF transduction (K18 fib and K19/PL fib, respectively). T: cellular fraction recovered in 1% Triton-X lysis buffer. S: Triton-insoluble fraction solubilized in 1% SDS lysis buffer. Equal proportions of Triton and SDS fractions were load on SDS-PAGE gels. Magnification: 40×. Scale bar: 50 μm.

FIG. 27A is an image demonstrating that one hour after the addition of myc-K18/P301L PFF/BioPORTER reagent complex to T40/P301L-transfected cells, a few rare cells showed local accumulation of insoluble tau (PHF-1 staining) that colocalized with exogenous pffs (myc staining). FIG. 27B is an image depicting ThS staining entirely overlapped with PFF staining (myc) 3 hr after PFF addition. 1% Triton-X100 was added during fixing to remove soluble proteins. Magnification: 40× for FIG. 27A; 20× for FIG. 27B. Scale bar: 10 μm in FIG. 27A; 100 μm in FIG. 27B.

FIG. 28A is an image depicting lysates from cells transiently transfected with T40/P301L mutant tau and treated with different doses of WGA. The immunoblots reveal that WGA did not dose-dependently increase the expression level of tau and there was no accumulation of Triton-insoluble tau without fibril transduction. T: 1% Triton-X fraction; S: 1% SDS fraction. Equal proportions of Triton and SDS fractions were loaded on SDS-PAGE. FIG. 28B is an image demonstrating that enhanced cellular association with PFFs induced by WGA was blocked by GlcNAc treatment. PFFs were recognized by polyclonal anti-myc Ab. Magnification: 40×. Scale bar: 50 μm.

FIG. 29, comprising FIG. 29A through FIG. 29D, is a series of images demonstrating that α-syn-hWT Pffs recruit endogenous α-syn in neuronal cultures to form pathologic, insoluble aggregates. Two weeks after pff addition, neurons were fixed with paraformaldehyde alone or paraformaldehyde with 1% Triton X-100 (Tx-100) to extract soluble proteins. FIG. 29A shows that in PBS-treated neurons, α-syn localized to the presynaptic terminal and was Tx-100 soluble. Addition of α-syn pffs formed Tx-100 insoluble aggregates which recruited α-syn away from synapses. Scale bar=20 μm. FIG. 29B depicts immunoblots from 2 independent studies in which neurons were treated with α-syn-hWT pffs and 2 weeks later were sequentially extracted with 1% Tx-100 followed by 2% SDS. Antibodies that either recognize the C-terminus of α-syn (top) or are specific for mouse α-syn (bottom) showed that in PBS-treated neurons, α-syn was soluble in Tx-100. α-syn-hWT pff treatment reduced soluble α-syn and increased Tx-100-insoluble α-syn. The first lane shows α-syn-hWT pffs alone to demonstrate that the C-terminal antibody recognizes both human and mouse α-syn, and the mouse specific antibody recognizes only mouse pffs. Furthermore, the α-syn-hWT pffs themselves are not phosphorylated. FIG. 29C shows that addition of α-syn pffs increased the accumulation of pathologic p-α-syn. Fixation with paraformaldehyde/Tx-100 demonstrated that the phosphorylated aggregates were insoluble. Scale bar=50 μm. Insert: Within the somata, aggregates appear as LB-like skein-like filaments and dense inclusions (arrow). PBS-treated neurons and neurons from α-syn −/− mice did not show p-α-syn. FIG. 29D shows that the phosphorylated aggregates are also ubiquitin positive (n=2). Scale bar=50 μm.

FIG. 30, comprising FIG. 30A and FIG. 30B, is a series of images demonstrating that a minimal domain of α-syn is necessary for aggregate formation. Pffs composed of full-length or indicated truncation α-syn mutants were added to DIV5 neurons and fixed 2 weeks later. FIG. 30A shows Tx-100 insoluble aggregates after addition of all constructs as detected by immunofluorescence using p-α-syn antibody. Scale bar=20 μm. α-syn-hWT or α-syn-mWT pffs were added to neurons on DIV5 and 2 weeks later were either fixed or extracted with 1% Tx-100 followed by 2% SDS. FIG. 30B depicts immunofluorescence and immunoblots which showed that α-syn-mWT pffs induced the appearance of phosphorylated, Tx-100-insoluble, α-syn. Data represent two independent experiments. Scale bar=20 μm.

FIG. 31, comprising FIG. 31A through FIG. 31F, is a series of images demonstrating the ultrastructure of the α-syn aggregates. FIG. 31A depicts transmission EM of α-syn-hWT pff-treated neurons showed filaments in the neuronal soma (see box highlight). FIG. 31B depicts immuno-EM of HRP-labeled p-α-syn inclusions were visualized in the neuronal soma near the nucleus. FIG. 31C shows filamentous inclusions in the neuronal soma that were labeled with nanogold particles. Insert: higher magnification of labeled filaments. FIG. 31D depicts presynaptic nanogold-labeled α-syn filaments. FIG. 31E shows a neuronal process with nanogold labeled α-syn filaments. FIG. 31F depicts HRP immunoreactivity for p-α-syn at a postsynaptic ending. Scale Bars: 1 μm (FIGS. 31A, 31B, 31C, and 31F) and 500 nm (FIG. 31D and FIG. 31E).

FIG. 32, comprising FIG. 32A through FIG. 32D, is a series of images demonstrating the time dependence of α-syn aggregate formation. α-syn-hWT pffs were added to DIV5 neurons, and fixed either 4, 7, or 10 days later. FIG. 32A (top row) shows that small puncta corresponding to neuritic p-α-syn were visible 4 days after pff addition, and by 7 days, neuritic p-α-syn levels increased, and accumulations were visible in some cell bodies. Ten days after addition of pffs, p-α-syn was seen throughout the neurites as small puncta, longer fibrous structures, and as somal accumulations. FIG. 32A (bottom row) shows that when α-syn-hWT pffs were added to DIV10 neurons when α-syn expression at the presynaptic terminal is higher, pathology progresses more quickly and are detectable at 2 days after pff addition and aggregates in the cell bodies as early as 4 days after pff addition. Scale bar=50 μm. FIG. 32B shows immunoblots of DIV5 neurons sequentially extracted with 1% Tx-100 and 2% SDS, 4, 7, 10, and 14 days after PBS or α-syn-hWT pff addition. Over time, soluble α-syn was reduced with a concomitant increase in total and p-α-syn in the Tx-100-insoluble fraction. FIG. 32C depicts double immunofluorescence for p-α-syn and the axonal marker, mouse tau (T49), and FIG. 32D depicts double immunofluorescence for p-α-syn and the dendritic marker, MAP2. P-α-syn predominantly colocalized with tau but not MAP2 4 days after pff addition. Two weeks after pff addition, aggregates were found in axons, cell bodies and dendrites where they colocalized with MAP2. Scale bar=20 μm

FIG. 33, comprising FIG. 33A through FIG. 33D, is a series of images demonstrating that α-syn-hWT pffs are internalized into neurons. FIG. 33A depicts labeling of extracellular and intracellular α-syn-hWT pffs. Live neurons were incubated with mAb Syn204 (red) to label extracellular α-syn-hWT pffs, followed by fixation, permeabilization, and incubation with LB509 (green) to label both intracellular and extracellular α-syn-hWT pffs. Extracellular α-syn-hWT pffs are visualized as yellow in the merged image. Arrowheads highlight examples of internal α-syn-hWT pffs (green). Scale bar=10 μm. FIG. 33B shows labeling of p-α-syn and α-syn-hWT pffs. Fixed and permeabilized neurons were double labeled with mAbs 81A (green) to detect p-α-syn and Syn 204 (red) to detect α-syn-hWT pffs. P-α-syn can be visualized accumulating from seeds of α-syn-hWT pffs. α-syn-hWT pffs were added to DIV5 neurons and fixed 14 days later. FIG. 33C shows immunofluorescence that was performed to label extracellular α-syn-hWT pffs (red) and p-α-syn (green). A sequential stack of confocal images shows that puncta corresponding to α-syn-hWT pffs colocalized with p-α-syn within a neurite, suggesting that pathologic p-α-syn grows from intracellular pffs. DIV5 neurons were treated with either α-syn-hWT pffs alone or pffs with 1 μg/mL or 5 μg/mL of WGA. To inhibit WGA endocytosis, neurons were preincubated with 0.1M GlcNAC followed by incubation with pffs, GlcNAC, and 5 μg/mL of WGA. Neurons were fixed 4 days later. FIG. 33D depicts immunoblots and immunofluorescence which showed that WGA dose-dependently increased the extent of insoluble p-α-syn.

FIG. 34, comprising FIG. 34A through FIG. 34H, is a series of images demonstrating intracellular propogation of pathologic α-syn aggregates. FIG. 34A is a schematic showing that hippocampal neurons grown in microfluidic chambers were interconnected by channels accessible only to neuronal processes. α-syn-1-120-myc pffs were added to the compartment containing exclusively neurites of DIV 5 neurons. FIG. 34B shows neurons stained with SNL-4 (total syn) 7 days after treatment with α-syn pffs showing the distribution of neurites, somata, and exogenous pffs which appear as large puncta in the neuritic compartment. P-α-syn is found within the microchannels as well as cell bodies in the somatic chamber, indicating propagation of α-syn pathology from neurites toward the somata. Inset shows high power images of the somatic (top) and neuritic (bottom) compartments. FIG. 34C and FIG. 34D depict α-syn-1-120-myc pff-treated neurons double-stained using anti-myc and syn202 (Syn) before (FIG. 34C) and after extraction with Tx-100 (FIG. 34D), demonstrating the presence of insoluble p-α-syn within neurons and processes. Myc-positive pffs were confined to the neuritic compartment. FIG. 34E depicts a schematic where α-syn-1-120-myc pffs were added to the somal-containing compartment of DIV5 neurons. FIG. 34F shows neurons immunostained for tau and p-α-syn showing pathology extending within axonal processes from the somal compartment into the neuritic compartment. FIG. 34G and FIG. 34H show α-syn-1-120-myc pff-treated neurons double-stained using anti-Myc and Syn202 before (G) and after extraction with Tx-100 (H). α-Syn within the axons is Tx-100-insoluble. α-Syn-1-120-myc pffs remained confined to the somal compartment and thus p-α-syn within the neuritic compartment resulted from propagation from the perikarya to the neurites. Scale bar=50 μm in (FIG. 34B) and 40 μm in (FIGS. 34C, 34D, 34F, 34G, and 34H).

FIG. 35, comprising FIG. 35A through FIG. 35C, is a series of images demonstrating the effects of aggregate formation on neuronal density and expression of synaptic proteins. Neurons were fixed 2 weeks after treatment with PBS or α-syn-hWT pffs. FIG. 35A shows that formation of α-syn aggregates caused recruitment of α-syn away from the presynaptic terminal such that it no longer colocalized with VAMP2. FIG. 35B depicts immunoblots for the indicated synaptic proteins from neurons 2 weeks after treatment with PBS or α-syn-hWT pffs and sequentially extracted with buffer containing 1% Tx-100 followed by 2% SDS. Equal amounts of protein were loaded in each lane. Band intensities were quantified and expressed as average percent change (±SEM) in protein levels from pff-treated neurons relative to PBS-treated neurons. *indicates p<0.05, ** indicates p<0.01. GAPDH, n=4; βsyn, n=3; CSPα, n=7; complexin, n=4, Synapsin I, n=3; Synapsin II, n=8; Snap25, n=8; VAMP2, n=6; Syntaxin I, n=7; Synaptophysin, n=7; Dynamin I, n=4; GlurI, n=4; PSD95, n=5. Neurons were fixed 4 (n=3), 7 (n=2), or 14 days (n=5) after addition of α-syn-hWT pffs or PBS. FIG. 35C depicts quantification of neuronal survival in neurons treated with α-syn-hWT pffs. Immunofluorescence was performed using NeuN to label neuronal nuclei. Numbers of nuclei were counted in cultures from WT neurons and α-syn −/− neurons (n=2). There was an approximately 40% decrease in cell number in α-syn-hWT pff-treated, WT, but not α-syn −/− neurons relative to PBS-treated controls only after 14 days of pff treatment.

FIG. 36, comprising FIG. 36A through FIG. 36F, is a series of images demonstrating the effect of aggregate formation on neural network activity in cultured neurons. FIG. 36A shows that PBS-treated WT neurons showed flickering events and simultaneous bursting. The spontaneous activity in α-syn-hWT pff-treated WT neurons showed reduced coordination and frequency of oscillations. FIG. 36B shows the level of coordinated spontaneous activity, quantified as the synchronization index. α-Syn-hWT pff-treated neurons (red) showed a significant decrease in synchronicity by day 4, relative to PBS-treated neurons (blue) and the deficit continued for longer treatment duration. Primary neurons from α-syn −/− mice treated with α-syn-hWT pffs (purple) did not show reductions in the synchronization index relative to PBS-treated neurons (green). FIG. 36C shows the excitatory tone in the network, as determined by recording spontaneous activity and forcing synchronous oscillations via network disinhibition with bicuculline. Incremental concentrations of NBQX were added until coordinated activity stopped and the excitatory tone was reported as [NBQX]/K_{d}. FIG. 36D shows that the excitatory tone in α-syn-hWT pff -treated neurons (red), but not PBS-treated neurons (blue), showed significant decreases by 10 as well as 14 days after pff treatment. Addition of α-syn-hWT pffs to α-syn −/− neurons did not affect excitatory tone. FIG. 36E shows functional network connectivity, derived from the rasters in FIG. 36A, depicted as nodes (neurons) of varying sizes, where the size of a given node is scaled to reflect the total number of connections to that particular node. FIG. 36F shows the average number of connections per neuron as determined from functional connectivity map. Compared to PBS, α-syn-hWT pff-treated neurons had fewer numbers of functional connections. Connectivity of α-syn-hWT pff-treated α-syn −/− neurons were similar to PBS-treated neurons. PBS-treated: day 4, n=9; day 7, n=11; day 10, n=10; day 14, n=9. PFF-treated: day 4, n=9; day 7, n=12; day 10, n=11; day 14, n=9.

FIG. 37, comprising FIG. 37A through FIG. 37C, is a series of images depicting neurons 2 weeks after α-syn-hWT pffs addition. FIG. 37A (upper panel) shows that α-syn-hWT pffs produced p-α-syn aggregates (labeled with mAb 81A) in neurites and somata of primary cortical neurons. FIG. 37A (lower panel) shows that α-Syn-hWT pffs-induced p-α-syn pathology in dopaminergic neurons (labeled with antibody to tyrosine hydroxylase) from mouse midbrain cultures. Scale bar=20 μm. FIG. 37B further shows that pathologic α-syn was not found in GFAP-positive astrocytes. Scale bar=50 μm. Wild type or α-syn −/− neurons were treated with PBS or α-syn-hWT pffs and 2 weeks later, were extracted with 1% Tx-100 followed by 2% SDS. FIG. 37C depicts immunoblots on these lysates performed with an antibody specific for mouse α-syn. α-syn −/− neurons did not show bands in either the Tx-100-soluble or Tx-100-insoluble fractions, demonstrating that α-syn in the insoluble fraction of pff-treated wild type neurons represents endogenous α-syn and not pffs. Immunoblot for GAPDH demonstrates the presence of protein in the α-syn −/− lysates.

FIG. 38 is an image demonstrating that p-α-syn pathological aggregates 2 weeks after treatment with varied concentrations of α-syn-hWT pffs. Wild type primary hippocampal neurons were treated with the following concentrations of α-syn-hWT pffs: 0.1, 1, 10, 100 ng/mL and fixed 2 weeks later. As low as 0.1 ng/mL of pffs can induce pathology in a subset of neurons. The amount of p-α-syn pathological aggregates increase progressively with higher concentrations of α-syn-hWT pffs. Scale Bar=50 μm.

FIG. 39 is an image depicting the co-labeling of α-syn and β-syn following addition of α-syn-hWT pffs. Wild type primary hippocampal neurons were treated with α-syn-hWT pffs and fixed with either paraformaldehyde alone or paraformaldehyde with Tx-100 to extract soluble proteins. Immunofluorescence was performed with β-syn antibody, mAb Syn207, and a rabbit polyclonal antibody, pSer 6.1, which recognizes p-α-syn. In both PBS and α-syn-hWT pff treated neurons, β-syn localizes to puncta at the presynaptic terminal and is Tx-100 soluble. Thus, pff treatment does not cause β-syn to redistribute to insoluble p-α-syn aggregates. Scale Bar=20 μm.

### DETAILED DESCRIPTION

The present invention relates generally to cellular models that provide a valuable research tool that can be exploited to identify molecules, gene products and intracellular pathways that affect either the formation or degradation of proteins involved in neurological diseases.

In one embodiment, the cellular model is based on the ability to produce fibrillar α-Syn inclusions in cultured cells that exhibit characteristics similar to authentic Lewy bodies (LBs). The intracellular α-Syn aggregation can be triggered by the introduction of exogenously produced recombinant α-Syn fibrils into cultured cells engineered to overexpress α-Syn. In another embodiment, intracellular α-Syn aggregation can be triggered by the introduction of exogenously produced recombinant α-Syn fibrils into cultured cells, wherein the cultured cells are not engineered to overexpress α-Syn. The exogenously produced recombinant α-Syn fibrils can be generated from full-length or truncated mammalian α-Syn. Unlike unassembled α-Syn, these α-Syn fibrils “seed” recruitment of endogenous soluble α-Syn protein and convert them into insoluble, hyperphosphorylated, and ubiquitinated pathological species. Thus, this α-Syn cell-based model recapitulates key features of LBs in diseased human brains.

In another embodiment, the cellular model is based on the ability to produce intracellular tau inclusions in cultured cells engineered to overexpress tau. That is, intracellular tau aggregation can be triggered by the introduction of exogenously produced pre-formed tau fibrils into cultured cells engineered to overexpress tau.

These cellular models (e.g., α-Syn and tau aggregation models) thus provide a valuable research tool that can be exploited to identify molecules, gene products and intracellular pathways that affect either the formation or degradation of neuropathological α-Syn and/or tau aggregates.

In one embodiment, the invention includes cells engineered to express α-Syn. In another embodiment, the invention includes cells engineered to express tau. Preferably, the cells are transduced with the desired gene using a cationic-liposome methodology optimized for intracellular delivery. In another embodiment, the invention includes cells that are not manipulated to express a given protein (e.g normal healthy cells) for use in cell based models for elucidating the pathological mechanisms underlying a number of diseases including but not limited to neurodegenerative diseases.

In another embodiment, the invention relates to compositions and assays to identify candidate agents and gene products that modulate tau protein or α-Syn aggregation. In one embodiment, the agents identified are therapeutically useful for the prevention and or treatment of Alzheimer's disease, Parkinson's disease, and other α-synuclein-associated and tau-associated diseases.

The invention also provides methods for identifying and characterizing modulators of protein filament formation. The methods are particularly useful for identifying those agents or gene products which inhibit or prevent protein filament formation within neurons of mammalian subjects, such as the formation of tau filaments in Alzheimer's patients, and α-synuclein filaments in Parkinson's patients. Thus, the mammalian subjects are preferably human subjects. According to the methods of the invention, protein monomers which are associated with formation of intra- or extra-cellular aggregates are combined under physiological conditions and the formation of protein aggregates is assessed in the absence or the presence of a test agent. A reduction in the size or stability of proteinaceous polymeric filaments, or their complete absence, in the presence of the test agent, as compared to what is observed in the absence of the test agent, is an indication that the test agent is an inhibitor of proteinaceous polymeric filament formation.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system.

Alternatively, the instructional material can be shipped separately from the container with the intention that the instructional material and the compound be used cooperatively by the recipient.

The terms “Lewy body” and “Lewy bodies” refer to abnormal aggregates of protein that develop in nerve cells. The primary protein aggregate in a Lewy body is composed of α-synuclein.

The term “microarray” refers broadly to both “DNA microarrays” and “DNA chip(s),” and encompasses all art-recognized solid supports, and all art-recognized methods for affixing nucleic acid molecules thereto or for synthesis of nucleic acids thereon.

“Modulation of α-synuclein aggregation” includes prevention, inhibition or decrease, change in time of onset, progress to or reversal of aggregation. Thus, “modulation” includes promoting the disaggregation of α-synuclein aggregates. The term “α-synuclein” includes mutant and wild-type polypeptides, and fragments thereof. An “activity” or “function” of α-synuclein includes, but is not limited to, formation of inclusions/aggregation in the cytoplasm, association with the cell membrane, interaction with an α-synuclein associated protein. In addition, α-synuclein can inhibit phospholipase D (PLD) activity, cause toxicity to cells, and lead to impaired proteasomal activity. For example, the identified agent may prevent α-synuclein misfolding, inhibit formation of α-synuclein inclusions/aggregation, or promote α-synuclein disaggregation. Accordingly, irrespective of the mechanism of action, agents identified by the screening methods described herein will provide therapeutic benefit to α-synuclein associated diseases.

“Modulation of tau aggregation” includes prevention, inhibition or decrease, change in time of onset, progress to or reversal of aggregation. Thus, “modulation” includes promoting the disaggregation of tau aggregates. The term “tau” includes mutant and wild-type polypeptides, and fragments thereof. An “activity” or “function” of tau includes, but is not limited to, formation of inclusions/aggregation in the cytoplasm, association with the cell membrane, interaction with a tau associated protein. The identified agent may prevent tau misfolding, inhibit formation of tau inclusions/aggregation, or promote tau disaggregation. Accordingly, irrespective of the mechanism of action, agents identified by the screening methods described herein will provide therapeutic benefit to tau associated diseases.

The term “neurodegenerative disease” as used herein, refers to a neurological disease characterized by loss or degeneration of neurons. Neurodegenerative diseases include neurodegenerative movement disorders and neurodegenerative conditions relating to memory loss and/or dementia. Neurodegenerative diseases include tauopathies and α-synucleopathies. Examples of neurodegenerative diseases include, but are not limited to, presenile dementia, senile dementia, Alzheimer's disease, Parkinsonism linked to chromosome 17 (FTDP-17), progressive supranuclear palsy (PSP), Pick's disease, primary progressive aphasia, frontotemporal dementia, corticobasal dementia, Parkinson's disease, Parkinson's disease with dementia, dementia with Lewy bodies, Down's syndrome, multiple system atrophy, amyotrophic lateral sclerosis (ALS) and Hallervorden-Spatz syndrome.

The term “neurofibrillary tangles” as used herein, refers to abnormal structures located in the brain and composed of dense arrays of paired helical filaments (neurofilaments and microtubules). Neurofibrillary tangles include tau proteins, particularly microtubule-associated tau proteins. The number of neurofibrillary tangles present in a brain is believed to correlate with the degree of dementia in the subject. Neurofibrillary tangles are a distinguishing characteristic of Alzheimer's disease.

“Naturally occurring” as used herein describes a composition that can be found in nature as distinct from being artificially produced. For example, a nucleotide sequence present in an organism, which can be isolated from a source in nature and which has not been intentionally modified by a person in the laboratory, is naturally occurring.

As used herein, “phenotypically distinct” is used to describe organisms, tissues, cells or components thereof, which can be distinguished by one or more characteristics, observable and/or detectable by current technologies. Each of such characteristics may also be defined as a parameter contributing to the definition of the phenotype. Wherein a phenotype is defined by one or more parameters an organism that does not conform to one or more of the parameters shall be defined to be distinct or distinguishable from organisms of the phenotype.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “tau protein” refers generally to any protein of the tau protein family. Tau proteins are characterised as being one among a larger number of protein families which co-purify with microtubules during repeated cycles of assembly and disassembly (Shelanski et al. (1973) Proc. Natl. Acad. Sci. USA, 70, 765-768), and are known as microtubule-associated-proteins (MAPs). Members of the tau family share the common features of having a characteristic N-terminal segment, sequences of approximately 50 amino acids inserted in the N-terminal segment, which are developmentally regulated in the brain, a characteristic tandem repeat region consisting of 3 or 4 tandem repeats of 31-32 amino acids, and a C-terminal tail.

The term “tauopathy” as used herein refers to a neurodegenerative disorder or disease involving the deposition of abnormal tau protein isoforms in neurons and glial cells in the brain. Taopathies include diseases and disorders in which tau proteins are abnormally phosphorylated, including tau protein which is hyperphosphorylated. Tauopathies include, but are not limited to, presenile dementia, senile dementia, Alzheimer's disease, Parkinsonism linked to chromosome 17 (FTDP-17), progressive supranuclear palsy (PSP), Pick's disease, primary progessive aphasia, frontotemporal dementia and corticobasal dementia.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition associated with alpha-synuclein and/or tau aggregation, including alleviating symptoms of such diseases.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to the development of cell-based models that provide significant insights into the events that regulate the formation of intracellular α-Syn and tau inclusions. The cell-based models of the invention represent an invaluable tool for further elucidating the pathological mechanisms underlying a number of diseases including but not limited to neurodegenerative diseases. The cell-based models include pre-formed α-Syn and/or tau fibrils exogenously applied to a cell population.

The invention is based, at least in part, on the discovery that that introduction of exogenously assembled α-Syn fibrils (e.g., seeded α-Syn fibrils) catalyzes intracellular α-Syn aggregation. The seeded α-Syn fibrils rapidly recruit endogenous soluble α-Syn protein, converting this into detergent-insoluble inclusions. Inclusions were found to occur both in cell populations that have and have not been engineered to overexpress α-Syn. Importantly, inclusions were formed when the exogenously applied α-Syn fibrils were generated from either full length or truncated α-Syn. This concept is consistent with the tau aggregation model of the invention where introduction of exogenously assembled tau PFFs (e.g., seed tau PFFs) catalyzes intracellular tau aggregation in a cell engineered to overexpress tau. The seeded tau PFFs rapidly recruit endogenous soluble tau protein, converting this into detergent-insoluble inclusions.

The cell-based model involves “seeding” a cell having been or not been engineered to overexpress α-Syn with exogenous α-Syn fibrils, where the seeded α-Syn fibrils serve as a nidus for the recruitment and association of endogenous α-syn. Similarly, the cell-based model involves “seeding” an engineered cell overexpressing tau with exogenous tau PFFs where the seeded tau PFFs serve as a nidus for the recruitment and associate of endogenous tau. These cell-based models are at least valuable in identifying molecules and gene products that modify intracellular pathways of α-syn and tau aggregation and degradation.

In one embodiment, the cell-based models of the invention further comprise the addition of wheat germ agglutinin (WGA) to the cell population. WGA binds N-acetyl-glucosamine and sialic acids at the cell surface and induces adsorptive-mediated endocytosis. Addition of WGA to the cell population increases the extent of α-Syn and tau aggregation.

In one embodiment, the cell-based models of the invention provide a system for developing agents that prevent the formation of nucleating cores (or block further aggregate growth from existing seed structures) as a therapeutic strategy for the treatment of patients with neurodegenerative protein misfolding diseases. This is because the cell-based models of the invention provides significant insights into the events that regulate the formation of intracellular α-syn and tau inclusions and represent an invaluable tool for further elucidating the pathological mechanisms underlying diseases associated with these types of inclusions.

In one embodiment, the cell-based models of the invention can be used to identify gene products that affect synuclein and/or tau inclusion formation. In turn, the identified gene product can be considered as a target gene for screening candidate agents that affect synuclein and/or tau inclusion formation. For example, a skilled artisan armed with the present disclosure would appreciate that the cell-based models can be used to screen for an siRNA to identify gene products that either increase or reduce inclusion formation. These gene products may then serve as drug targets to modulate synuclein or tau inclusions.

### Alpha-Synuclein

Parkinson's disease is a neurodegenerative disorder that is pathologically characterized by the presence of intracytoplasmic Lewy bodies (Lewy in Handbuch der Neurologie, M. Lewandowski, ed., Springer, Berlin, pp. 920-933, 1912; Pollanen et al., J. Neuropath. Exp. Neurol. 52:183-191, 1993), the major components of which are filaments consisting of α-synuclein (Spillantini et al., Proc. Natl. Acad. Sci. USA 95:6469-6473, 1998; Arai et al., Neurosc. Lett. 259:83-86, 1999), a 140-amino acid protein (Ueda et al., Proc. Natl. Acad. Sci. U.S.A. 90:11282-11286, 1993).

Accumulation of α-synuclein is also a cytopathological feature common to Lewy body disease and multiple system atrophy (Wakabayashi et al, Acta Neuropath. 96:445-452, 1998; Piao et al, Acta Neuropath. 101:285-293, 2001). Multiple system atrophy is a sporadic neurodegenerative disease in adults characterized by neuronal and glial cytoplasmic inclusions, containing α-synuclein.

Parkinson's disease α-synuclein fibrils, like the Aβ fibrils of Alzheimer's disease, also consist of a predominant beta-pleated sheet structure. It is therefore believed that compounds found to inhibit Alzheimer's disease Aβ amyloid fibril formation might also be effective in the inhibition of α-synuclein fibril formation. These compounds would therefore also serve as therapeutics for Parkinson's disease, in addition to having efficacy as a therapeutic for Alzheimer's disease and other amyloid disorders.

α-Synucleinopathies are conditions associated with the aggregation of α-synuclein and include Parkinson's disease, LB variant Alzheimer's disease, multiple system atrophy (MSA), LB dementia and Hallervorden-Spatz disease.

The compositions and methods disclosed herein use a protein comprising an alpha-synuclein polypeptide. The term “alpha synuclein” encompasses naturally occurring alpha synuclein sequences (e.g., naturally occurring wild type and mutant alpha synucleins) as well as functional variants thereof.

Unless otherwise apparent from the context, reference to α-Syn or its fragments includes the natural amino acid sequence indicated above, or fragments thereof, as well as analogs including allelic, species and induced variants. Amino acids of analogs are assigned the same numbers as corresponding amino acids in the natural sequence when the analog and human sequence are maximally aligned. Analogs typically differ from naturally occurring peptides at one, two or a few positions, often by virtue of conservative substitutions. Some natural allelic variants are genetically associated with hereditary LBD. The term “allelic variant” is used to refer to variations between genes of different individuals in the same species and corresponding variations in proteins encoded by the genes.

α-Syn, its fragments, and analogs can be synthesized by solid phase peptide synthesis or recombinant expression, or can be obtained from natural sources.

Automatic peptide synthesizers are commercially available from numerous suppliers, such as Applied Biosystems, Foster City, Calif. Recombinant expression can be in bacteria, such as E. coli, yeast, insect cells or mammalian cells. Procedures for recombinant expression are described by Sambrook et al., Molecular Cloning: A Laboratory Manual (C.S.H.P. Press, NY 2d ed., 2002).

In some embodiments, a full-length alpha-synuclein protein can be used. The term “full-length” refers to an alpha-synuclein protein that contains all the amino acids encoded by the alpha-synuclein cDNA. In other embodiments, different lengths of the alpha-synuclein protein may be used. For example, only functionally active domains of the protein can be used. Thus, a protein fragment of almost any length can be employed, provided it is functional. Mammalian α-Syn, that is α-Syn protein native to any mammal, may be used in the invention.

Also described herein are methods of preparing and transferring nucleic acids encoding an alpha-synuclein protein into a mammalian cell so that the cell expresses the alpha-synuclein protein. The term “alpha synuclein nucleic acid” encompasses a nucleic acid comprising a sequence of wild type alpha synuclein as well as a nucleic acid encoding any of the variants of alpha-synuclein described herein.

In certain embodiments, fusion proteins including at least a portion of the alpha-synuclein protein may be used. For example, a portion of the alpha-synuclein protein may be fused with a second domain. The second domain of the fusion protein can be selected from the group consisting of: an immunoglobulin element (e.g., an Fc fragment of an immunoglobulin molecule), a dimerizing donlain, a targeting domain, a stabilizing domain, and a purification domain. Alternatively, a portion of alpha-synuclein protein can be fused with a heterologous molecule such as a detection protein. Exemplary detection proteins include: (1) a fluorescent protein such as green fluorescent protein (GFP), cyan fluorescent protein (CFP) or yellow fluorescent protein (YFP); (2) an enzyme such as β-galactosidase or alkaline phosphatase (AP); and (3) an epitope such as glutathione-S-transferase (GST) or hemagluttin (HA). To illustrate, an alpha synuclein protein can be fused to GFP at the N- or C-terminus or other parts of the alpha-synuclein protein. These fusion proteins provide methods for rapid and easy detection and identification of the alpha-synuclein protein in the recombinant host cell, exemplified herein by the mammalian cell (e.g., a mammalian neuronal cell).

Given the naturally unfolded state of alpha-synuclein, it is believed that even highly elevated levels of α-Syn overexpression in cultured cells may not generate sufficient amounts of oligomeric or protofibrillar nuclei required to seed fibril elongation. Thus, in one embodiment, the present invention relates to the introduction of exogenously assembled α-Syn fibrils into a cell engineered to overexpress α-Syn which results in the catalysis of intracellular α-Syn aggregation in the cell. That is, the α-Syn fibrils introduced into the cell rapidly recruits endogenous soluble α-Syn protein and is converted into detergent-insoluble inclusions. In another embodiment, exogenously assembled α-Syn fibrils are introduced into a cell that is not engineered to overexpress α-Syn or any other protein. An advantage of generating inclusions following the method of the invention results in a pathological hyperphosphorylated and ubiquitinated α-Syn species thereby recapitulating key features of human LBs in a cell culture model. Accordingly, in one embodiment, the invention provides a method of generating α-Syn into insoluble forms having features typical of intracellular LB-like inclusions.

The cell-based model for α-Syn aggregation is able to recapitulate key features of LBs in human diseased brain. Therefore, the cell-based model of the invention is useful for understanding the pathology of diseases associated with α-Syn. In addition, the cell-based model is useful for screening agents that promote α-synuclein disaggregation and/or prevent aggregation. The α-syn aggregation model in which exogenous α-syn fibrils are introduced into cells can be used to screen for potential α-synuclein aggregation modulating activity. For example, the α-syn aggregation model can be used to screen for agents that prevent, inhibit or reverse aggregation.

An advantage of the cell-based model of the invention, in which exogenous α-syn fibrils are introduced into cells, is that the “seeded” intracellular fibrils serve as a nidus for the recruitment and association of endogenous α-syn, with the latter being phosphorylated and ubiquitinated as in LBs. Thus, the cell model of the invention recapitulates key features of LBs in human PD brains.

In one embodiment, initial screening methods described herein may include any assay that provides for the identification of agents or gene products that prevent α-synuclein misfolding, inhibit formation of α-synuclein inclusions/aggregation or promote α-synuclein disaggregation. Detection of the level of α-synuclein aggregation in such systems may be measured by immunostaining or fluorescence detection (e.g., Thioflavin T). (See, e.g., Sung et al., (2005) J. Biol. Chem.: 280 (25216-24). In other embodiments, electron microscopy will be used to visualize fibrillation and/or aggregation. Additionally, detergent extraction and centrifugation can be utilized in some embodiments to separate soluble and insoluble protein, followed by SDS-PAGE electrophoresis. In other embodiments atomic force microscopy images can be processed and viewed.

Other embodiments contemplate screening assays using fluorescence activated cell sorting (FACS) analysis. FACS is a technique well known in the art, and provides the means for scanning individual cells for the presence of fluorescently labeled/tagged moiety. The method is unique in its ability to provide a rapid, reliable, quantitative, and multiparameter analysis on either living or fixed cells. For example, a synuclein can be suitably labeled using a variety of fluorescent tags (e.g., ThioflavinT, fluorescent antibodies, etc.) providing a useful tool for the analysis and quantitation of α-synuclein aggregation and fibril and/or aggregate formation.

In some embodiments, a cell free in vitro assay can be followed by or substituted with an in vitro primary culture assay. In this assay, primary neuronal cultures consisting of pure dopaminergic neurons, mixed primary cultures with dopaminergic neurons and glial cells made to express or overexpress wild-type or mutant α-synuclein or a neuronal cell line stably transfected with a wild-type or mutant α-synuclein can be utilized for screening.

### Tau

Neuritic plaques, neurofibrillary tangles (NFTs), and neuropil threads are hallmark lesions of Alzheimer's disease (AD) that contain filamentous intraneuronal inclusions of tau protein (Buee et al., Brain Res. Rev. 33: 95-130 (2000)). Because tau filaments form in brain regions associated with memory retention, and because their appearance correlates well with the degree of dementia, they have emerged as robust markers of disease progression (Braak et al., Acta. Neuropathol. (Berl) 87: 554-567 (1991); Braak et al., Acta Neuropathol. (Berl) 87: 554-567 (1994)). Tau filaments also appear in other neurodegenerative tauopathies, including Pick's disease and corticobasal degeneration, with the neuronal populations affected being disease dependent (Feany et al., Ann. Neurol. 87: 554-567 (1996)). Thus tau filament formation heralds the onset of cytoskeletal disorganization that is characteristic of degenerating neurons, and may represent a fundamental pathobiological response of neurons to various insults.

NFTs have long been recognized as the hallmarks of Alzheimer's disease and the existence of a close correlation between the presence and distribution of NFTs and the degree of cognitive impairment in Alzheimer's disease further emphasizes the critical role of tau pathology in the development of the disease. Hyperphosphorylated tau proteins tend to dissociate from microtubule and assemble into paired helical filaments. Other factors proposed to facilitate the aggregation of tau include oxidation, polyanions, and nucleation. In vitro tests have demonstrated that all tau isoforms are able to aggregate, however, tau fragments containing the repeat domain exhibit faster kinetics in in vitro assembly tests. Thus, not wishing to be bound by a theory, fragmentation of tau could be a significant factor that enhances the aggregation of tau and causes the generation of tangle like structures.

Accumulation of early-stage oligomeric aggregated tau species is associated with the development of functional deficits during the pathogenic progression of tauopathy, and accumulation of pre-fibril granular oligomers correlates with Braak staging in post-mortem analysis of AD brains (Berger Z, et al. (2007) J. Neurosci. 27(14): 3650-62; Maeda S, et al. (2006) Neuroscience Res. 54:197-201). The role of misfolded tau in AD has been shown in a number of studies using antibodies specific for tau conformational epitopes. Levels of conformationally altered tau in postmortem neocortical specimen increased with progressing dementia in AD (Haroutunian V, et al. (2007) Neurobiology of Aging, 28: 1-7). The involvement of soluble tau as the primary causative factor of neurotoxicity in AD is supported by these findings. Tau has also been found to associate with Aβ (beta amyloid plaque) in brain tissue, and this interaction is thought to facilitate the aggregation of these proteins (Guo J-P et al. (2006) PNAS 103:1953-1958).

Tau protein exists in six isoforms of amino acid residues 352-441 in the adult brain (Goedert et al. (1989) Neuron, 3, 519-526). The term “tau protein” refers to any protein of the tau protein family including, but not limited to, native tau protein monomer, precursor tau proteins, tau peptides, tau intermediates, metabolites, tau derivatives that can be antigenic, or antigenic fragments thereof. Fragments include less than entire tau protein provided the fragment is antigenic and will cause antibodies or antibody binding fragments to react with the tau fragment.

The amino acid sequences of tau protein isoforms are also described in Ballatore C, Lee V M, Trojanowski J Q, Nat Rev Neurosci. 2007 September; 8(9):663-72. Review; Hasegawa M., Neuropathology. 2006 October; 26(5):484-90; and D'Souza I, and Schellenberg G D, Biochim Biophys Acta. 2005 Jan. 3; 1739(2-3):104-15. These references, mentioned in the specification, are incorporated herein by reference for all that they disclose.

In some cases, mutations in the gene encoding Tau (sometimes called MAPT) cause tauopathies, particularly in frontotemporal dementia and Parkinsonism linked to chromosome 17 (FTDP-17) and other frontotemporal dementias. Many FTDP-17 mutations decrease binding to microtubules in vitro and/or increase their propensity to form fibrils. Other tauopathy-associated mutations alter the splice pattern of Tau to generate predominantly 3R or 4R Tau. Yet another class of Tau mutations on the N-terminus alters the ability to bind to dynactin. All of these mutations have the potential to interfere with normal functions of Tau.

The present invention provides a cell model that is able to recapitulate features of tauopathies and therefore can be used to study the causes and consequences of tau aggregation. To date there are few cell models with robust tau aggregation. It is believed that no desirable cell model exists because high expression of tau over-stabilizes MTs and inhibits cell division, and is therefore not well tolerated by dividing cultured cells (Kanai, et al., 1989, J. Cell Biol. 109:1173-1184, Vogelsberg-Ragaglia, et al., 2000, Mol. Biol. Cell. 11:4093-4104). Moreover, because tau is a highly soluble protein, overexpressed tau resists aggregation despite spontaneous hyperphosphorylation in most cell lines. Prior to the present invention, the understanding of tau fibrillization has relied on studies using cell-free systems in which the formation of tau amyloid fibrils is greatly enhanced by polyanionic factors (Goedert, et al., 1996, Nature. 383:550-553; Kampers, et al., 1996, FEBS Lett. 399:344-349; Chirita, et al., 2003, J. Biol. Chem. 278:25644-25650). It has been shown that the MT-binding repeats of tau are both necessary and sufficient for in vitro fibrillization, and the repeat domain alone assembles into fibrils more readily than full-length tau (Wille, et al., 1992, J. Cell Biol. 118:573-584). Importantly, tau fibril assembly occurs by a nucleation-dependent mechanism, whereby the formation of oligomeric intermediates constitutes an initial lag phase followed by a relatively rapid elongation phase (Friedhoff, et al., 1998, Proc. Natl. Acad. Sci. U.S.A. 95:15712-15717).

Contrary to the prior art, the present invention is directed to “seeding” tau fibrillization reactions with preformed tau fibrils (tau PFFs) which bypasses the rate-limiting nucleation step and accelerates fibrillization of monomeric tau. Accordingly, the invention provides a method of “seeding” to promote tau aggregation in cultured cells.

Accordingly, the tau aggregation cell-based model of the present invention is based on a similar concept as the α-syn cell-based model of the invention discussed elsewhere herein. The tau aggregation model is based on the introduction of exogenous pre-formed tau fibrils (tau PFFs) into cells that overexpress tau, preferably a tau having the P301L mutation. In some instances, the internalized tau PFFs are in a state where the tau protein is misfolded, thereby recruiting endogenous tau that is expressed by the engineered cells. This results in the aggregation feature that resembles NFTs and neuropil threads.

In other instances, internalization of minute quantities of tau PFFs can recruit endogenous tau into detergent-insoluble aggregates, which can be enhanced by engineering the cells to have the P301L tau mutation. Using the P301L-expressing cells as a model for tau aggregation is useful for a research tool because the cells exhibit tau inclusions that resemble morphologically tau tangles in tauopathies in a mammal.

Cells expressing the P301L mutation can be isolated from a non-human transgenic animal that has been engineered to express a recombinant gene coding for P301L mutant tau. However, other tau gene sequences having a mutation linked to tau pathology may also be expressed as a transgene in the animal. Preferably, the animal is a mammal, in particular a rodent such as a mouse, a guinea pig, or a rat. An alternative to isolating a cell from a transgenic animal engineered to express a recombinant gene coding for P301L mutant tau, normal cells can be engineered to express a recombinant gene coding for P301L mutant tau or other tau gene sequences having a mutation linked to tau pathology.

In one embodiment, the tau aggregation cell-based model of the present invention is based on the introduction of exogenous pre-formed tau fibrils (tau PFFs) into cells that stably overexpress tau preferably the tau has the P301L mutation. In some instances, the internalized tau PFFs are in a state where the tau protein is misfolded thereby recruiting endogenous tau that is expressed by the engineered cells. This results in the aggregation feature that resembles NFTs and neuropil threads.

In one embodiment, the invention provides a cell-based model comprising a cell expressing a recombinant gene encoding tau protein, preferably tau that has the P301L mutation. The cell is capable of producing NFTs. In another embodiment, the cell that expresses a recombinant gene encoding tau protein is capable of producing an increased number of NFTs. In yet another embodiment, that cell that expresses a recombinant gene encoding tau protein is capable of accelerated production of NFTs. In another embodiment, the cell produces NFTs which are comparable to those commonly found in human neurodegenerative diseases.

According to the present invention, the cell based-model is used to screen a compound or modulatory agent for the ability of the compound or modulatory agent to modulate the formation of NFTs, and/or the formation of neuropil threads. Determining the formation of NFTs in the cell-based model can be accomplished using a variety of methods, for instance immunocytochemistry and electron microscopy, and it can comprise the utilization of conformation-dependent antibodies that are capable of recognizing and discriminating a tau molecule in the context of NFTs from tau molecules existing in other states of aggregation.

In a preferred embodiment, the conformation-dependent antibodies are optically labeled, preferably flourescently labeled, and can be detected, for instance, using a number of optical methodologies such as fluorescence polarization spectroscopy, fluorescence correlation spectroscopy, fluorescence cross-correlation spectroscopy, fluorescence intensity distribution analysis, fluorescence lifetime measurements, fluorescence anisotropy measurements, fluorescence resonance energy transfer, or combinations thereof. Alternatively, in another embodiment, antibodies that are capable of recognizing phosphorylated epitopes of tau, in particular epitopes whose degree of phosphorylation correlates with the state of tau aggregation and NFT formation as observed in Alzheimer's disease, in particular the tau epitope S422, can be used.

The invention also provides methods for testing the efficacy of compositions, including small molecules, siRNA, shRNA and/or peptides, to block, reverse or inhibit protein, e.g., tau, aggregation in a cell-based system. For example, a neuronal cell line can be used in the context of the tau aggregation cell-based system to assess aggregation, growth and survival of cells.

### Compositions

The invention provides compositions comprising protein aggregation inhibitors, pharmaceutical compositions comprising them, and methods for making and using them, including methods for preventing, reversing, slowing or inhibiting protein aggregation, e.g., for treating diseases that are characterized by protein aggregation—including some degenerative neurological diseases and conditions, such as Parkinson's disease, Alzheimer's Disease (AD), Lewy body disease (LBD) and Multiple system atrophy (MSA). In one aspect, the compositions of the invention specifically target synuclein, beta-amyloid and/or tau protein aggregates, and the methods of the invention can be used to specifically prevent, reverse, slow or inhibit synuclein, beta-amyloid and/or tau protein aggregation.

In alternative embodiments, the compositions and methods of the invention, including the synuclein, beta-amyloid and/or tau protein aggregation inhibiting (including aggregation-preventing, or aggregation-reversing) compositions of the invention, and the pharmaceutical compositions comprising them, are used to treat, prevent or ameliorate (including slowing the progression of) degenerative neurological diseases related to or caused by aggregation, e.g., alpha-synuclein, beta-amyloid and/or tau protein aggregation. In one aspect, the compositions and methods of this invention are used to treat, prevent or ameliorate (including slowing the progression of) Parkinson's disease, Alzheimer's Disease (AD), Lewy body disease (LBD) and Multiple system atrophy (MSA).

The invention provides methods and assays of screening for a candidate therapeutic agent (drug, compound or any of the other therapeutic agents named herein) and identifying an agent for treating a condition or disease associated with alpha-synuclein and/or tau aggregation. A “candidate agent” as used herein, is any substance with a potential to reduce, reverse, interfere with or block alpha-synuclein and/or tau aggregation. Various types of candidate agents may be screened by the methods described herein, including nucleic acids, polypeptides, small molecule compounds, and peptidomimetics.

Candidate agents include chemicals (including polymers, organic compounds, etc.); therapeutic molecules (including therapeutic drugs, antibiotics, etc.); biomolecules (including hormones, cytokines, proteins, lipids, carbohydrates, cellular membrane antigens); receptors (neural, hormonal, nutrient, and cell surface receptors) or their ligands; and viruses (including retroviruses, herpes viruses, adenoviruses, lentiviruses, etc.).

Candidate agents may be screened from large libraries of synthetic or natural compounds. One example is an FDA approved library of compounds that can be used by humans. In addition, synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.), and a rare chemical library is available from Aldrich (Milwaukee, Wis.). Combinatorial libraries are available and can be prepared. Alternatively, libraries of natural compounds in the form of bacterial, fungal, plant and animal extracts are also available, for example, Pan Laboratories (Bothell, Wash.) or MycoSearch (NC), or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. It should be understood, although not always explicitly stated that the agent can be used alone or in combination with another modulator, having the same or different biological activity as the agents identified by the subject screening method. Several commercial libraries can immediately be used in the screens.

Candidate agents may include a small molecule. Examples of small molecules include, but are not limited to, small peptides or peptide-like molecules (e.g., a peptidomimetic). As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics.

In other embodiments, candidate agents also encompass numerous chemical classes, though typically they are organic molecules, often small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl, sulphydryl or carboxyl group.

### Pharmaceutical

The invention also provides methods and systems for identifying those agents that may be used in a therapeutic composition for treating a disorder characterized by protein aggregation—including some degenerative neurological diseases and conditions, such as Parkinson's disease, Alzheimer's Disease (AD), Lewy body disease (LBD) and Multiple system atrophy (MSA). In one aspect, the compositions of the invention specifically target synuclein, beta-amyloid and/or tau protein aggregates, and the methods of the invention can be used to specifically prevent, reverse, slow or inhibit synuclein, beta-amyloid and/or tau protein aggregation.

In one embodiment, the invention also provides methods and systems for identifying those agents that may be used in a therapeutic composition for treating a disorder associated with alpha-synuclein and/or tau aggregation.

In certain embodiments, candidate agents (i.e., drugs or compounds) may be formulated in combination with a suitable pharmaceutical carrier. Such formulations comprise a therapeutically effective amount of the agent, and a pharmaceutically acceptable carrier (excipient). Examples of suitable carriers are well known in the art. To illustrate, the pharmaceutically acceptable carrier can be an aqueous solution or physiologically acceptable buffer. Optionally, the aqueous solution is an acid buffered solution. Such acid buffered solution may comprise hydrochloric, sulfuric, tartaric, phosphoric, ascorbic, citric, fumaric, maleic, or acetic acid. Alternatively, such carriers include, but are not limited to, saline, buffered saline, dextrose, water, glycerol, ethanol, and combinations thereof. Formulations will suit the mode of administration, and are well within the skill of the art.

In certain embodiments of such methods, one or more agents can be administered, together (simultaneously) or at different times (sequentially). In addition, such agents can be administered with another type(s) of drug(s) for treating a disease associated with alpha-synuclein and/or tau aggregation. For example, the identified agent may be administered together with Levodopa (L-DOPA) for treating Parkinson's disease.

The dosage range depends on the choice of the agent, the route of administration, the nature of the formulation, the nature of the subject's condition, and the judgment of the attending practitioner. Wide variations in the needed dosage, however, are to be expected in view of the variety of drugs available and the differing efficiencies of various routes of administration. For example, oral administration would be expected to require higher dosages than administration by intravenous injection. Variations in these dosage levels can be adjusted using standard empirical routines for optimization, as is well understood in the art.

Various delivery systems are known and can be used to administer a biologically active agent of the invention, e.g., encapsulation in liposomes, microparticles, microcapsules, expression by recombinant cells, receptor-mediated endocytosis (see, e.g., Wu and Wu, (1987), J. Biol. Chem. 262:4429-4432), construction of a therapeutic nucleic acid as part of a retroviral or other vector, etc. Methods of delivery include but are not limited to intra-arterial, intra-muscular, intravenous, intranasal, intracerebral and oral routes. In a specific embodiment, it may be desirable to administer the pharmaceutical compositions of the invention locally to the area in need of treatment; this may be achieved by, for example, and not by way of limitation, local infusion during surgery, by injection, by means of a catheter, or implantation of a device coated with the agent and a slow-release mechanism. In certain embodiments, the agents are delivered to a subject's nervous systems, preferably the central nervous system. In another embodiment, the agents are administered to neuronal tissues undergoing, or suspected of undergoing alpha-synuclein and/or tau deposition.

Administration of the selected agent can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician.

The preparation of pharmaceutical compositions of this invention is conducted in accordance with generally accepted procedures for the preparation of pharmaceutical preparations. See, for example, Remington's Pharmaceutical Sciences 18th Edition (1990), E. W. Martin ed., Mack Publishing Co., PA. Depending on the intended use and mode of administration, it may be desirable to process the active ingredient further in the preparation of pharmaceutical compositions. Appropriate processing may include mixing with appropriate non-toxic and non-interfering components, sterilizing, dividing into dose units, and enclosing in a delivery device.

Pharmaceutical compositions for oral, intracerebral, intramuscular, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or aerosol when used with an appropriate nebulizer device.

Pharmaceutically acceptable liquid compositions can, for example, be prepared by dissolving or dispersing an agent embodied herein in a liquid excipient, such as water, saline, aqueous dextrose, glycerol, or ethanol. The composition can also contain other medicinal agents, pharmaceutical agents, adjuvants, carriers, and auxiliary substances such as wetting or emulsifying agents, and pH buffering agents.

Where desired, the pharmaceutical compositions can be formulated in slow release or sustained release forms, whereby a relatively consistent level of the active compound are provided over an extended period.

The present invention further comprises a method of treating a mammal, preferably a human, having a disease characterized by protein aggregation—including some degenerative neurological diseases and conditions, such as Parkinson's disease, Alzheimer's Disease (AD), Lewy body disease (LBD) and Multiple system atrophy (MSA). In one embodiment, the disease is associated with alpha-synuclein and/or tau aggregation.

Certain embodiments of the invention provide a pharmaceutical preparation/dosage formulation provided in the form of a transdermal patch and formulated for sustained release formulation, in a therapeutically effective amount sufficient to treat a disease associated with filament aggregation in a patient, wherein the dosage formulation, when administered (provided as a patch) to the patient, provides a substantially sustained dose over at least about 2 hours, 4 hours, 6 hours, 8, hours, 12 hours, 20 hours, or at least about 24 hours.

The pharmaceutical compositions described herein can be prepared alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrasternal injection, bolus injections, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those that comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 0.1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles that comprise the active ingredient and that have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, and the like. Preferably, the compound is, but need not be, administered intraperitoneally.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Exogenous α-Synuclein Fibrils Seed the Formation of Lewy Body-Like Intracellular Inclusions in Cultured Cells

Cytoplasmic inclusions containing α-synuclein (α-Syn) fibrils, referred to as Lewy bodies (LBs), are the signature neuropathological hallmarks of Parkinson's disease (PD). Although α-Syn fibrils can be generated from recombinant α-Syn protein in vitro, the production of fibrillar α-Syn inclusions similar to authentic LBs in cultured cells has not been achieved. The results presented herein demonstrate that intracellular α-Syn aggregation can be triggered by the introduction of exogenously produced recombinant α-Syn fibrils into cultured cells engineered to overexpress α-Syn. Unlike unassembled α-Syn, these α-Syn fibrils “seeded” recruitment of endogenous soluble α-Syn protein and their conversion into insoluble, hyperphosphorylated, and ubiquitinated pathological species. Thus, this cell model recapitulates key features of LBs in human PD brains. Also, the results presented herein support the concept that intracellular α-Syn aggregation is normally limited by the number of active nucleation sites present in the cytoplasm and that small quantities of α-Syn fibrils can alter this balance by acting as seeds for aggregation.

The materials and methods employed in these experiments are now described.

### Materials and Methods

Recombinant α-Syn Proteins and Protein Labeling

Recombinant WT, Myc-tagged, and mutant human α-Syn proteins used in this study are detailed in Table 1. α-Syn proteins were purified from Escherichia coli BL21-RIL cells expressing α-Syn constructs from the PRK172 expression vector. Purification by gel filtration and ion exchange chromatography were performed as previously described (Murray et al., 2003 Biochemistry 42:8530-8540; Luk et al., 2008 Biochemistry 47:12614-12625). The purity of α-Syn proteins were verified by Coomassie blue staining and the concentrations of each determined by using a BCA assay (ThermoFisher) with BSA as a standard. To generate fluorescently tagged α-Syn protein (α-SynAlexa594), monomeric WT α-Syn was covalently labeled with the succinimidyl ester formofAlex_-Fluor 594 (Invitrogen) as described elsewhere (Luk et al., 2007 Biochemistry 46:12522-12529). Conjugation reactions were titrated to achieve a dye:protein molar ratio of ≈1:10.

TABLE 1
Recombinant proteins used

| Protein | Comments |
|---|---|
| α-Syn WT | Full-length α-Syn |
| α-Syn-Myc | α-Syn, C-terminal Myc tag |
| α-Syn Δ71-82 | Assembly deficient |
| α-Syn1-120 | Lacks final 20 residues on C terminus, |
| | accelerated fibril assembly |
| α-Syn21-140 | Lacks first 20 residues on N terminus |
| α-Syn S129A | Ser-129 mutated to Ala; no phosphorylation |
| | possible at residue 129 |

Fibril Assembly

Fibrils were prepared in reactions (200 μL per tube) containing 360 M (≈5 mg/mL) α-Syn monomer in assembly buffer (50 mM Tris/100 mM NaCl, pH 7.0). Reactions were incubated at 37° C. with constant agitation (1,000 rpm) in an orbital mixer. Fluorescent fibrils were assembled by including 1.8 M α-Syn⁵⁹⁴ in WT α-Syn assembly reactions. Reactions were stopped after 5 days, aliquoted, and stored at −80° C. until use. The presence of amyloid fibrils was confirmed by using thioflavin fluorimetry and electron microscopy (EM).

Cell Culture and Fibril Transduction in Mammalian Cells

Cells stably expressing α-Syn used in this study are listed in Table 2. Generation of stable QBI-HEK-293 (QBiogene) and SH-SY5Y cell lines is as follows. Expression vectors for mutant human α-Syn were generated from pcDNA3.1_/WT-α-Syn by using PCR site-directed mutagenesis (SLIM method) (Chiu et al., 2004 Nucleic Acids Res 32(21):e174). QBI-HEK-293 cells (QBiogene) stably expressing WT or mutant α-Syn were maintained in full media (DMEM, 10% FBS) supplemented with G418 (1000 g/mL; Gibco), penicillin/streptomycin, and L-glutamine. Stable cell lines used in this study are listed in Table 2. SH-SY5Y cells stably expressing α-Syn (Mazzulli et al., 2006 J Neurosci 26:10068-10078) were maintained in MEM (containing 10% FBS, L-glutamine, 1 mM sodium pyruvate, penicillin/streptomycin, 300 μg/mL G418). Untransfected HeLa cells were maintained in the same media without G418. HeLa cells were transiently transfected with pcDNA3.1+/WT-α-Syn by using Lipofectamine-2000 (Invitrogen) as per manufacturer's instructions.

QBI cells were plated in 35-mm tissue culture plates and allowed to reach 80-90% confluence for transduction experiments. Cells were transferred to serum-free media 1 h before transduction. For each plate, 10 μL of cationic-liposomal protein transduction reagent (Bioporter; Sigma) was added to a 1.5-mL Microfuge tube and evaporated as per the manufacturer's guidelines. The resulting reagent dry-film was then directly resuspended with 80 μL of PBS containing α-Syn fibrils (100 μg/mL) fragmented by brief sonication with a hand-held probe. Protein:reagent complexes were allowed to form at room temperature for 10 min, after which the mixture was further diluted in OptiMEM (Invitrogen) and added to cells. Cells were then further incubated for 4 h, washed twice with Versene and 0.5% trypsin/EDTA to remove extracellular α-Syn fibrils, and transferred onto 6-well tissue culture plates or poly(d-lysine)-coated glass coverslips. Transduced cells were maintained in media containing 0.5% FBS unless otherwise indicated.

TABLE 2
Stable cell lines used

| Designation | Cell type | Description |
|---|---|---|
| QBI-Syn-WT | QBI/293 | Full-length α-Syn |
| QBI-Syn-A53T | QBI/293 | α-Syn A53T |
| QBI-Syn-Δ71-82 | QBI/293 | α-Syn Δ71-82 |
| QBI-Syn-S129A | QBI/293 | α-Syn S129A |
| QBI-Syn-Myc | QBI/293 | Full-length α-Syn |
| | | (C-terminal Myc-tag) |
| SY5Y-Syn-WT | SH-SY5Y | Full-length α-Syn |
| SY5Y-Syn-Δ71-82 | SH-SY5Y | α-Syn Δ71-82 |

Immunocytochemistry and Thioflavin (Th)S Staining

Fluorescence immunocytochemistry was performed by using primary antibodies listed in Table 3. Cells on coverslips were washed with PBS and fixed with 4% paraformaldehyde. For staining of microtubules and cytoskeletal proteins, cells were fixed briefly with 0.3% glutaraldehyde in PEM buffer and extracted with 1% Triton X-100. To visualize cell membranes, PHA-L conjugated to Alexa Fluor 488 (2 μg/mL; Invitrogen) was added to cells at 4° C. for 15 min before fixation. Fixed cells were blocked in 3% BSA and 3% FBS and permeabilized with 0.01% saponin. Markers of interest were identified by using specific antibodies against as listed in Table 3. Staining was revealed with appropriate Alexa Fluor 488 or Alexa Fluor 594 conjugated secondary antibodies (Invitrogen). For ThS staining, fixed cells were incubated with 0.05% ThS for 15 min and differentiated with 70% ethanol. Cells were counterstained with DAPI to reveal nuclei. Epifluorescence images were obtained by using a Nikon Eclipse 3000 inverted microscope equipped with a CoolSNAP HQ camera (Roper Scientific) and analyzed by using Metamorph 6.0 (Molecular Devices). High-power images were deconvoluted by using AutoQuant X (Media Cybernetics). Confocal laser scanning microscopic images were obtained by using a Zeiss LSM510 microscope and analyzed by using Volocity software (Improvision).

Immunoblot Analysis and Immunoprecipitation

For sequential extractions, cells were washed twice with PBS and scraped into ice-cold lysis buffer (50 mM Tris/150 mM NaCl/1% Triton X-100, pH 7.6) containing protease and phosphatase inhibitors. Lysates were cleared after sonication by centrifugation at 100,000×g for 30 min. Pellets were sonicated in lysis buffer, cleared, and the resulting insoluble material was resuspended in lysis buffer containing 1% SDS. Samples were separated on SDS/15% polyacrylamide gels, transferred to nitrocellulose membranes, and blocked in 5% nonfat milk in TBS. For phosphorylation-specific antibodies, Protein-Free block (ThermoFisher) was used instead of milk. Primary antibodies (Table 3) were detected with horseradish peroxidase-conjugated secondary antibodies (Jackson ImmunoResearch), and revealed by using Luminal (PerkinElmer Life Sciences). For immunoprecipitation, SDS-soluble lysate (100 g total protein) was cleared by incubation with 25 μL of A/G protein beads (Santa Cruz Biotechnology) for 1 h at 4° C. Samples were then transferred to fresh A/G protein beads (50 μL), which had been preincubated with 5 μg of anti-Myc antibody (9E10). Beads were incubated at 4° C. for 4 h and washed three times with cold RIPA buffer, and bound proteins were eluted with sample buffer containing 2% SDS.

TABLE 3
Antibodies used

| Antibody | Antigen | Comments | Host species | Dilution |
|---|---|---|---|---|
| SNL-1 | α-Syn | Raised against residues | Rabbit | 1:1,000 (WB, ICC) |
| | | 104-119 | polyclonal | |
| SNL-4 | α-Syn | N terminus, residues | Rabbit | 1:2,000 (WB, ICC) |
| | | 2-12 | polyclonal | |
| LB509 | α-Syn | C terminus, human | Monoclonal | 1:5,000 (WB); |
| | | specific | | 1:2,000 (ICC |
| Syn211 | α-Syn | C terminus, human | Monoclonal | 1:1,000 (ICC) |
| | | specific | | |
| Syn303 | α-Syn | N terminus, conformation | Monoclonal | 1:1,000 (ICC) |
| | | specific | | |
| Syn506 | α-Syn | N terminus, conformation | Monoclonal | 1:1,000 (ICC) |
| | | specific | | |
| Syn514 | α-Syn | N terminus, conformation | Monoclonal | 1:1,000 (ICC) |
| | | specific | | |
| 81A (α-Syn^{pSer129}) | α-Syn | Phosphorylation at | Monoclonal | 1:1,000 (WB); |
| | | Ser-129 | | 1:10,000 (ICC) |
| SPA-810 | Hsp70 | Heat shock protein 70 | Monoclonal | 1:1,000 (ICC) |
| SPA-835 | Hsp90 | Heat shock protein 90 | Monoclonal | 1:200 (ICC) |
| SPA-400 | Hsp40 | Heat shock protein 40, | Monoclonal | 1:1,000 (ICC) |
| | | Hdj-1 | | |
| 1D3 | Protein disulflde | ER chaperone | Monoclonal | 1:500 (ICC) |
| | isomerase | | | |
| GM130 | Golgi | Golgi matrix marker | Monoclonal | 1:500 (ICC) |
| DM1A | α-Tubulin | — | Monoclonal | 1:1,000 (ICC) |
| TU-02 | γ-Tubulin | — | Monoclonal | 1:500 (ICC) |
| 1DB4 | Lysosomal associated | — | Monoclonal | 1:250 (ICC) |
| | membrane protein 1 | | | |
| | (LAMP1) | | | |
| V6630 | Vimentin | — | Monoclonal | 1:1,000 (ICC) |
| 9E10 | c-Myc | c-Myc tag | Monoclonal | 1:1,000 (WB, ICC) |
| Anti-c-Myc | c-Myc | c-Myc tag | Rabbit, | 1:1,000 (ICC) |
| | | | polyclonal | |
| Ubi | Ubiquitin | Poly-ubiquitin | Rabbit, | 1:500 (ICC) |
| | | | polyclonal | |

Electron Microscopy (EM)

Transduced cells were seeded onto 25-mm Thermanox coverslips (Nunc) and fixed at various time intervals after addition of fibril:reagent complexes. For routine EM, cells were fixed in 0.1 M cacodylate buffer (pH 7.4) containing 1% paraformaldehyde, 2.5% glutaraldehyde, and 0.002% CaCl2. Cells were then washed in cacodylate buffer, postfixed for 1 h in 0.05M cacodylate containing 1% OsO4 and 1.5% potassium ferrocyanide, dehydrated in ethanol, and embedded in Araldite. Sections were stained with uranyl acetate and bismuth subnitrate. For immuno-EM, cells were fixed in periodate-lysine-paraformaldehyde (McLean et al., 1974 J Histochem Cytochem 22:1077-1083) for 4 h, washed several times in 50 mM NH₄Cl in PBS, and stored in 10% sucrose in PBS containing 0.02% NaN3. Antibody dilutions were prepared in PBS with 2% fish gelatin (PBS-FG) with 0.0005% saponin. Cells were permeablized for 30 min in PBS-FG with 0.05% saponin and incubated overnight in anti-Myc (9E10) antibody. Cells were then incubated for 3 h in biotinylated horse anti-mouse IgG (Vector Laboratories), followed by ABC Elite for 2 h (Vector Laboratories). After 15-min fixation in 1.5% glutaraldehyde (in 0.1 M cacodylate buffer, 5% sucrose, pH 7.4; Tris-S), cells were washed in 50 mM Tris, pH 7.6, and incubated in 0.2% DAB containing 10 mM imidazole in Tris-S for 15 min without H2O2 and for 20 min with 0.01% H2O2 added. Grids containing μ-Syn fibrils were negatively stained with 1% uranyl acetate as previously described (Luk et al., 2007 Biochemistry 46:12522-12529). Transmission EM images were collected using a Jeol 1010 electron icroscope at the University of Pennsylvania Biomedical Imaging Core.

The results of the experiments are now described.

### Intracellular α-Syn Fibrils, but not Soluble α-Syn Species, Seed the Formation of LB-Like Inclusions

To effectively serve as seeds for aggregation, exogenously added α-Syn fibrils must first localize to cellular compartments accessible to soluble endogenous α-Syn and persist for a sufficient period to allow recruitment of endogenous α-Syn and promote growth of inclusions. Recent studies have revealed that fibrils comprised of polyglutamine repeats, as well as of tau protein accumulating in various neurodegenerative disorders, can be internalized by cells on addition to culture medium (Ren et al., 2009, Nat Cell Biol. 11:219-U232; Frost et al., 2009, J Biol. Chem. 284:3546-3551), although the mechanism by which fibrils reach the cytoplasm is not clearly understood.

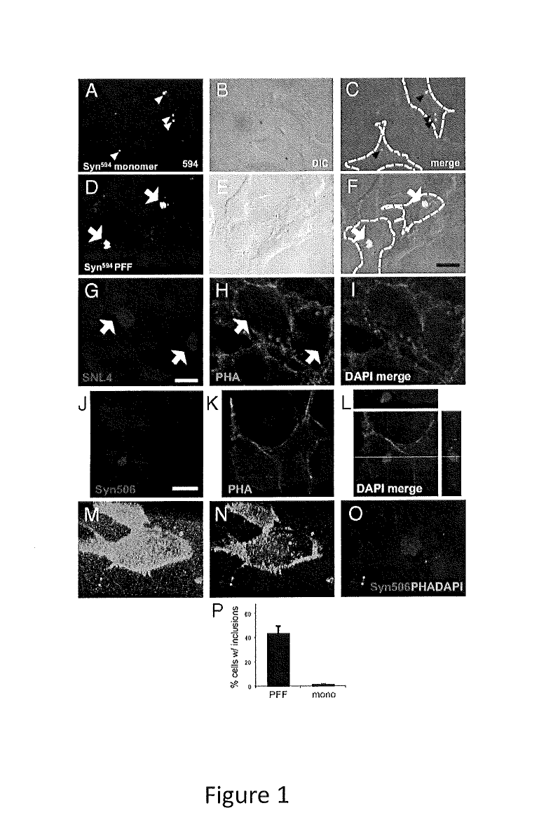

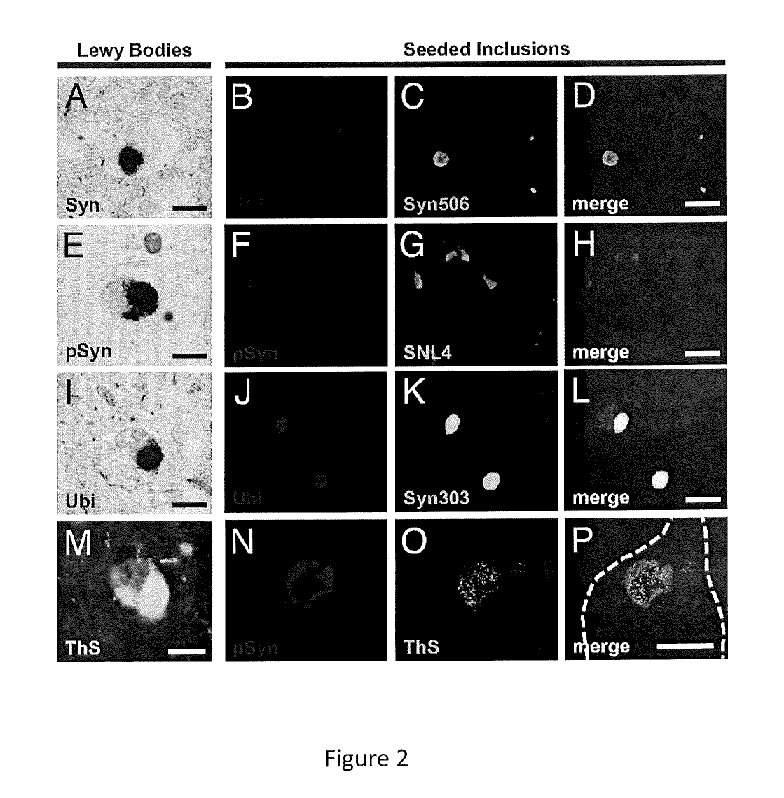

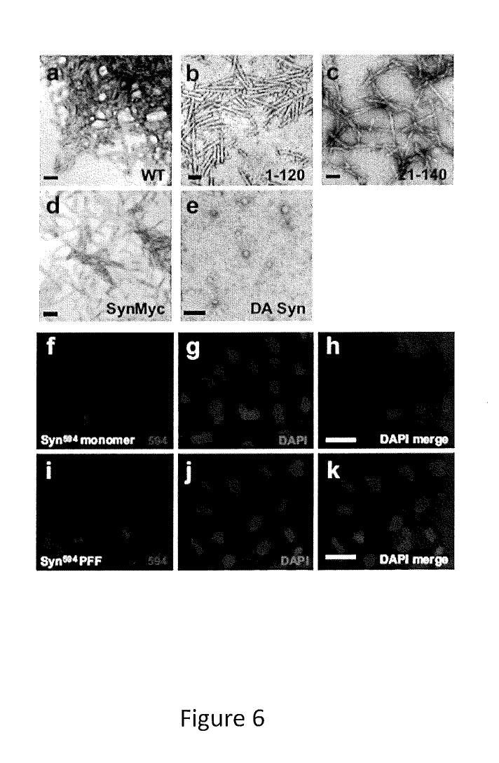

Experiments were designed to investigate whether monomeric and fibrillar forms of fluorescently labeled α-Syn (α-Syn⁵⁹⁴) could be efficiently introduced into the cytoplasm of QBI-293 cells to generate a cell culture model system of LB formation. In contrast to prior studies (Ren et al., 2009, Nat Cell Biol. 11:219-U232; Frost et al., 2009, J Biol. Chem. 284:3546-3551; Desplats et al., 2009, Proc Natl Acad Sci USA. 106:13010-13015), meaningful internalization of either monomeric or fibrillar α-Syn⁵⁹⁴ by mere addition of protein preparations to the culture medium were unsuccessful. However, intracellular α-Syn⁵⁹⁴ was readily detectable in cells by fluorescence- and differential interference contrast (DIC)-microscopy 24 h after transduction using cationic-liposomes optimized for intracellular protein delivery (FIG. 1A-1F; FIG. 6F-6K) (Zelphati et al., 2001, J Biol. Chem. 276:35103-35110). Transduced α-Syn⁵⁹⁴ monomers were transiently distributed throughout the cytoplasm as small punctate structures, whereas internalized α-Syn⁵⁹⁴ preformed-fibrils (PFFs) were present as large irregular foci. Although nearly all monomeric α-Syn⁵⁹⁴ was degraded by 48 h, α-Syn⁵⁹⁴ PFFs persisted for days within cells. When unlabeled WT α-Syn PFFs (FIG. 6A) were transduced into QBI-293 cells stably expressing WT α-Syn (QBI-WT-Syn cells), large cytoplasmic inclusions were observed within 48 h by using multiple antibodies against α-Syn that differed from the smaller irregular foci observed shortly after PFF transduction (FIGS. 1G-1L and FIG. 2) (Clayton et al., 1999, J Neurosci Res. 58:120-129). Confocal microscopy confirmed that these inclusions resided within the boundaries of the plasma membrane (FIG. 1J-1O). Similar results were also obtained in cells expressing a disease-associated α-Syn mutants (QBI-A53T-Syn) (Polymeropoulos et al., 1997, Science. 276:2045-2047).

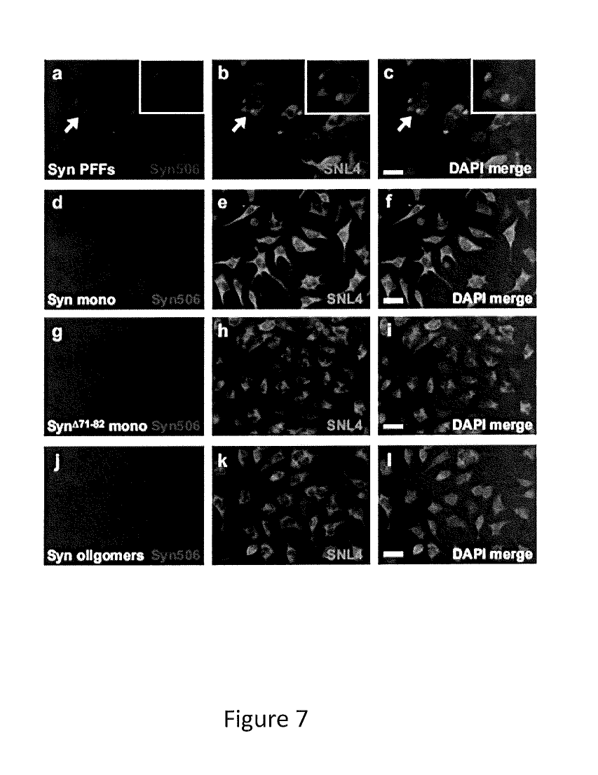

Inclusions were detectable as early as 24 h after PFF treatment and typically round in shape, ranging from 2 to 5 μm in diameter (FIGS. 1 and 2). More than 40% of PFF-transduced QBI-WT-Syn cells harbored these LB-like inclusions (FIG. 1P). Notably, only one inclusion was observed per cell, and, similar to LBs in PD, inclusions abutted or indented the nucleus in most cells. Interestingly, no inclusions were detected in cells transduced with nonfibrillar α-Syn comprised of either monomers or oligomers generated by incubation with dopamine (FIG. 6E and FIGS. 7D-7L) despite previous reports that certain species of oligomeric α-Syn may initiate intracellular α-Syn aggregation (Danzer et al., 2007, J. Neurosci. 27:9220-9232). Similarly, transduction with an unrelated soluble protein (β-galactosidase) did not result in inclusion formation. Thus, exogenously generated α-Syn fibrils specifically induce intracellular inclusion formation.

### Fibril-Seeded Intracellular α-Syn Inclusions Display Properties of LBs in Human Disease

Immunostaining of PFF-transduced QBI-WT-Syn cells with pan-α-Syn antibodies (e.g., SNL1 and SNL4) revealed intensely stained inclusions surrounded by diffuse cytoplasmic α-Syn, indicating that α-Syn was a major constituent of these aggregates (FIGS. 2B and 2G). Inclusions were also specifically labeled with antibodies recognizing misfolded forms of α-Syn (Syn506 and Syn303; FIGS. 2C and 2K), suggesting that α-Syn present within inclusions assumes pathological conformations similar to α-Syn in LBs of PD, but distinct from normal cellular α-Syn.

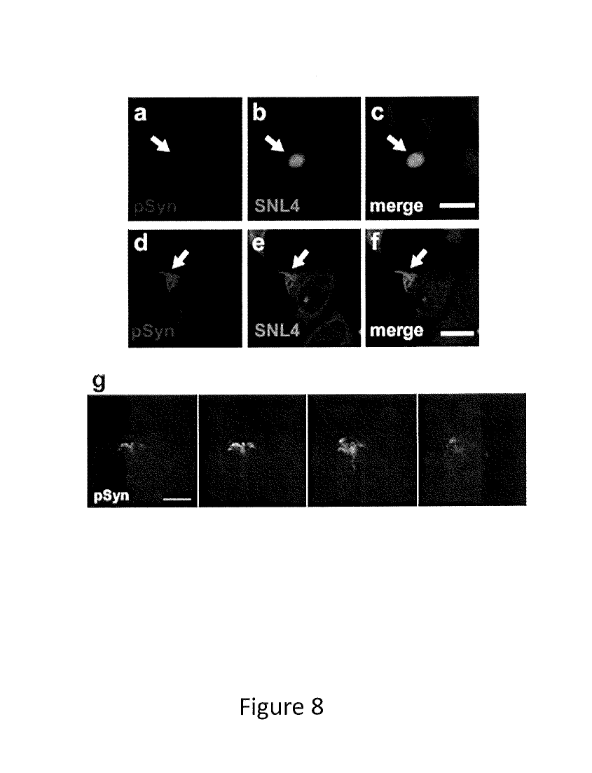

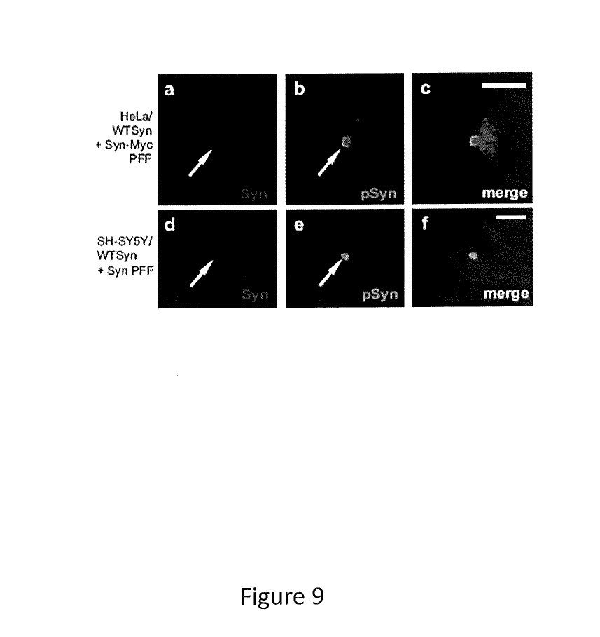

To further confirm that transduced α-Syn PFFs are intracellular and to determine whether the resulting seeded-inclusions recapitulate posttranslational modifications found in human LBs, the next series of experiments were performed to assess whether α-Syn within inclusions inside transduced cells underwent hyperphosphorylation (Fujiwara et al., 2002, Nat Cell Biol. 4:160-164) and ubiquitination (Spillantini et al., 1997, Nature. 388:839-840). Dual immunofluorescence with anti-α-Syn^{pSer129} revealed that nearly all inclusions showed strong immunoreactivity for α-Syn phosphorylated at Ser-129, thereby resembling authentic LBs in PD (FIGS. 2E-2H; FIG. 8). Similar phosphorylated α-Syn inclusions also formed after transduction with α-Syn PFFs in other cell lines overexpressing α-Syn, including HeLa and SH-SY5Y neuroblastoma cells (FIG. 9), indicating that this phenomenon extends to multiple cell types. The majority of the α-Syn inclusions were also ubiquitinated, as evidenced by ubiquitin-immunostaining (FIG. 2I-2L). Because recombinant α-Syn PFFs are neither phosphorylated or ubiquitinated before transduction, these modifications occur de novo after the transduction process and further confirm the intracellular location of α-Syn inclusions, as well as their verisimilitude to α-Syn fibrils found in PD brains. Staining with the amyloid-specific dye thioflavin S (ThS) revealed that most inclusions contained significant β-pleated sheet content like that found in authentic LBs (FIGS. 2M-2P). Together, these observations indicate that the intracellular α-Syn inclusions in the cell culture system of the invention closely resemble LBs in PD with respect to their key defining features.

### Exogenous Fibrils Seed Intracellular Inclusion Formation Via Recruitment and Conversion of Soluble Cytoplasmic α-Syn

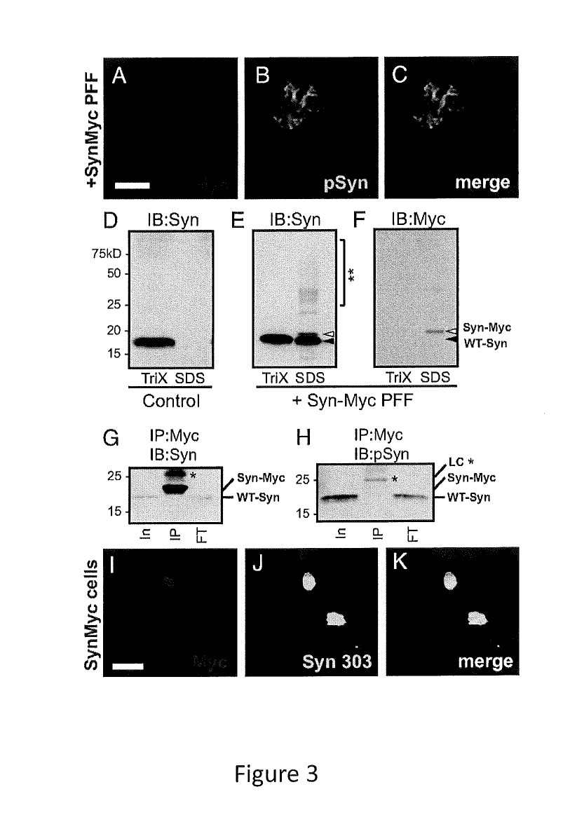

Given the substantial size of the intracellular inclusions within fibril-transduced cells and the observation that anti-α-Syn^{pSer129} primarily labeled the inclusion periphery, it was surmised that endogenously expressed α-Syn is recruited to the sites of internalized α-Syn PFFs. To test this hypothesis, and to determine the relative contributions of exogenous and cellular α-Syn to the formation of intracellular inclusions, QBI-α-Syn-A53T cells were transduced with fibrils assembled from recombinant Myc-tagged α-Syn (FIG. 6D). Double-immunofluorescence with anti-Myc (labeling the internalized α-Syn PFFs) and anti-α-Syn^{pSer129} revealed that Myc-positive PFF seeds are located at the center of inclusions (FIGS. 3A-3C). Also, Myc-labeled cores were surrounded by a region labeled with α-Syn^{pSer129} that was devoid of Myc staining, indicating that this phosphorylated α-Syn, which represents a significant proportion of inclusions, was comprised largely, if not exclusively, of endogenous α-Syn. Indeed, the lack of co-localization between Myc and α-Syn^{pSer129} suggested that the α-Syn PFF seeds were not significantly phosphorylated after internalization.

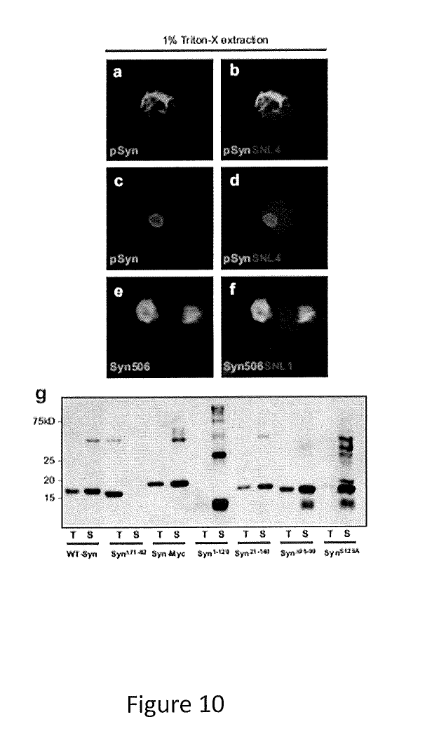

The α-Syn within LBs isolated from human PD brains has been demonstrated to be insoluble (Giasson et al., 2002, Neuron. 34:521-533; Baba et al., 1998, Am J Pathol. 152:879-884). Endogenous α-Syn from untransduced QBI-WT-Syn cells was highly soluble and entirely recovered after 1% Triton X-100 extraction (FIG. 3D). However, transduction with α-Syn-Myc PFFs led to the appearance of a significant amount of Triton-insoluble WT α-Syn as well as higher molecular weight species, which required SDS for solubilization (FIG. 3E). A weaker band consistent with α-Syn-Myc was also recovered in the SDS fractions of α-Syn-Myc PFF-transduced cells, the identity of which was confirmed on reprobing blots with anti-Myc antibody (FIGS. 3E and 3F). The relative intensities of the endogenous α-Syn band, which are several fold-higher than α-Syn-Myc in the SDS-soluble fraction from transduced cells (FIG. 3E), further support that the intracellular aggregates are comprised largely of recruited endogenous α-Syn. The insolubility of endogenous α-Syn recruited by α-Syn-Myc PFFs was also apparent by immunofluorescence after Triton X-100 extraction of transduced QBI-WT-Syn cells, which effectively removed all diffuse cytoplasmic α-Syn while leaving inclusions intact as shown by immunostaining with anti-α-Syn^{pSer129} or Syn506 (FIGS. 10A-10F).

### Inclusion Formation is Mediated by the Core Amyloid-Forming Region of α-Syn.

Concordant with the co-localization data, phosphorylation of Myc-tagged α-Syn PFFs after immunoprecipitation with anti-Myc and probing with α-Syn^{pSer129} antibody was not significantly detected, further confirming that exogenously introduced α-Syn fibrils are not phosphorylated (FIGS. 3G and 3H). Consistent with the majority of α-Syn found within detergent-insoluble inclusions representing newly recruited protein of cellular origin, cells stably expressing α-Syn-Myc also formed Myc-positive inclusions after transduction with WT α-Syn fibrils (FIGS. 3I-3K).

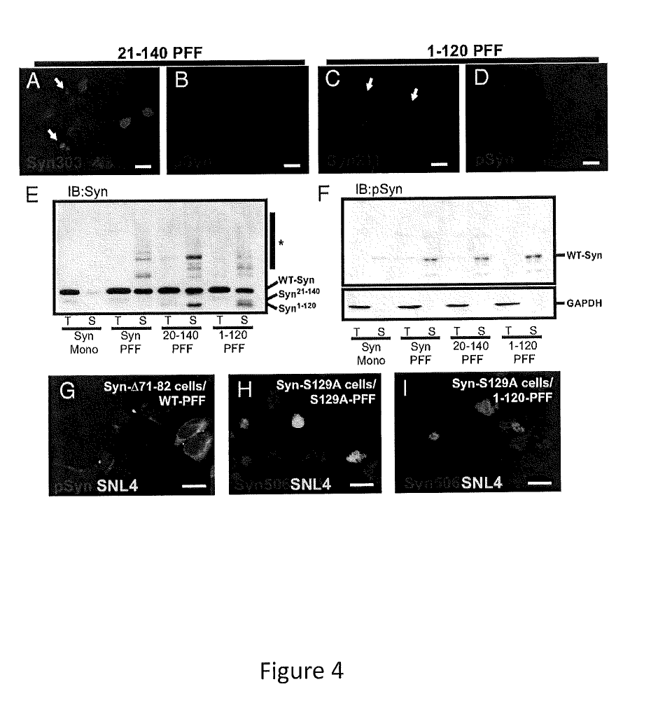

To better understand the molecular interaction between recruited endogenous α-Syn and the PFFs, QBI-A53T-Syn cells were transduced with fibrils lacking either the N-terminal (α-Syn²¹⁻¹⁴⁰) or C-terminal (α-Syn¹⁻¹²⁰) region of α-Syn. These PFFs are indistinguishable from WT α-Syn PFFs on EM evaluation (FIGS. 6A-6C and FIG. 10G). Transduction with N or C terminus truncated α-Syn PFFs resulted in robust formation of inclusions that were detected by antibodies that recognizing only endogenous full-length α-Syn. For example, Syn303 recognizes an N-terminal epitope not present in α-Syn²¹⁻¹⁴⁰ PFFs (FIGS. 4A and 4B), whereas α-Syn¹⁻¹²⁰ PFFs lack the epitope detected by anti-α-Syn^{pSer129} (FIGS. 4C and 4D). The abundance of endogenous α-Syn in the Triton-insoluble fractions from these cells confirms that recruitment has a central role in α-Syn inclusion formation (FIG. 4E).

Interestingly, phosphorylated α-Syn was found only in the Triton-insoluble fraction, which parallels studies of pathological α-Syn in PD brains (FIG. 4F). The ability of truncated α-Syn PFFs to seed further aggregate formation implies that the central portion of α-Syn is sufficient for the recruitment and subsequent incorporation of α-Syn into inclusions. Residing within this region is a hydrophobic sequence (residues 71-82) that forms the core of α-Syn fibrils (Giasson et al., 2001, J Biol. Chem. 276:2380-2386). To test whether this segment is required for the association of endogenous α-Syn, WT α-Syn PFFs were transduced into cells stably expressing α-Syn lacking this region. As predicted based on previous α-Syn fibrillization studies (Giasson et al., 2001, J Biol. Chem. 276:2380-2386), inclusions in transduced QBI-α-Syn^{Δ71-82} cells (FIG. 4G) were unable to be detected, thereby confirming that binding of endogenous α-Syn to PFFs requires the region of α-Syn critical for fibrillization in vitro.

The observation that phosphorylated α-Syn was present only in the Triton-insoluble fraction suggested that this modification occurs after recruitment to the growing inclusion. To confirm that phosphorylation of α-Syn at Ser-129 is not necessary for recruitment, either α-Syn¹⁻¹²⁰ or α-Syn^{S129A} PFFs were transduced into cells stably expressing phosphorylation-incompetent α-Syn^{S129A} Inclusions resembling those formed with WT α-Syn were detected, further revealing that phosphorylation is not required for inclusion seeding nor the subsequent recruitment of endogenous α-Syn (FIGS. 4H and 4I).

### Ultrastructure of Fibril-Seeded α-Syn Inclusions.

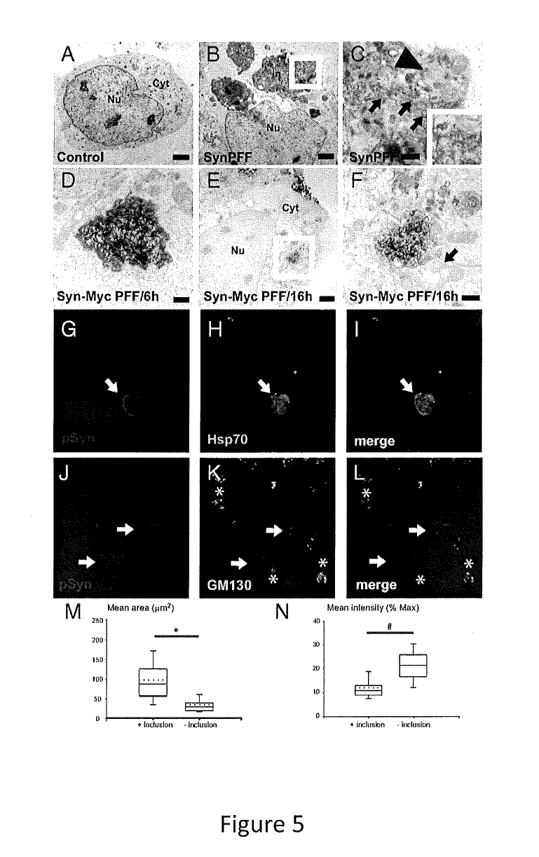

Cells containing α-Syn inclusions were next examined by EM. In monomer-transduced QBI-Syn-A53T cells, major organelles appeared intact and the cytoplasm was clear of any large accumulations (FIG. 5A). In contrast, electron-dense cytoplasmic inclusions were prominent after PFF-transduction (FIG. 5B). Inclusions were perinuclear, although no disruption of the nuclear membrane was observed. Examination at higher magnifications revealed the presence of distinct fibrillar structures ≈10-15 nm in diameter located at the center of inclusions (FIG. 5C). Inclusions also contained many vesicles, of which some were multilamellar and in contact with the fibrillar core. Immuno-EM was used to further monitor the localization of transduced α-Syn-Myc PFFs in QBI-Syn-A53T cells and to analyze the recruitment and incorporation of cellular α-Syn to fibrils. At 6 h after transduction, anti-Myc staining revealed accumulations of exogenous fibrils inside the cytoplasm within close proximity of the nucleus (FIG. 5D), where nearly all inclusions are subsequently located. Uranyl acetate counterstaining also revealed that transduced fibrils at this stage were not associated with vesicles typical of mature inclusions. By 16 h, PFF-transduced cells contained Myc-positive structures that were surrounded by various membrane bound organelles comparable with the arrangement observed in EM samples from cells obtained at later times (FIGS. 5e and 5F). These findings further verify that exogenously introduced PFFs reach the cytoplasmic space where they serve as a nidus for the formation of complex intracellular inclusions. Also, cytoplasmic vesicles appear to associate with fibrils during the inclusion formation process similar to what occurs in human LBs (Soper et al., 2008, Mol Biol Cell. 19:1093-1103). Because the immunofluorescence data presented herein indicate that insoluble α-Syn within inclusions extends considerably beyond the PFF core (FIGS. 3A-3C), these vesicles may contain hyperphosphorylated α-Syn and, thus, form an integral part of inclusions.

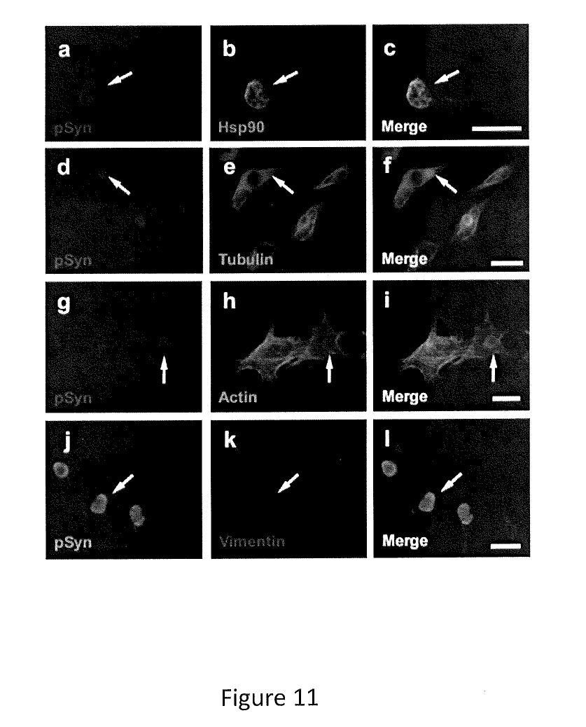

The consequences of LB-like α-Syn inclusions on cellular architecture and organization were next examined. Although colocalization of α-Syn^{pSer129}-positive inclusions were not detected with various cytoskeletal markers, inclusions were positive for Hsp70 and Hsp90 (FIGS. 5G-5I; FIG. 11), which are members of the heat-shock protein family widely reported to accumulate in LBs and misfolded protein inclusions in multiple neurodegenerative disorders. In contrast to phosphorylated α-Syn, which was distributed primarily at the periphery of inclusions but excluded from the core, Hsp70 was present throughout inclusions, suggesting that chaperones recognize both α-Syn PFF seeds and subsequently recruited endogenous α-Syn.

The next set of experiments were designed to examine whether the presence of LB-like inclusions in QBI-Syn-A53T cells disrupts trafficking pathways, including ER-Golgi transport and secretory function as recently shown in yeast models of α-Syn overexpression (Soper et al., 2008, Mol Biol Cell. 19:1093-1103; Cooper et al., 2006, Science. 313:324-328). Immunostaining for α-Syn^{pSer129} and the Golgi matrix protein GM130 in α-Syn PFF-transduced cells revealed that the majority of α-Syn inclusions were located near the cis-Golgi (FIGS. 5J-5L). Significantly, GM130 staining in inclusion-bearing cells exhibited a dispersed pattern compared with the dense stacked morphology in cells without inclusions. Quantitative analyses of the mean Golgi area and intensity also revealed significant differences between cells with and without α-Syn inclusions (FIGS. 5M and 5N). Thus, as in yeast models of synucleinopathies, seeded α-Syn inclusions alter normal cellular processes.

### Intracellular Inclusion Formation

The results presented herein demonstrate that intracellular inclusion formation in α-Syn overexpressing cells can be initiated by the presence of fibrillar α-Syn seeds. Once inside cells, fibrillar seeds actively recruit and convert soluble endogenous α-Syn into a misfolded state, leading to the formation and growth of detergent-insoluble structures closely resembling LBs in the brains of patients with PD and other synucleinopathies. Importantly, the α-Syn inclusions in the cell culture model of the invention also undergo several modifications characteristic of human LBs, including hyperphosphorylation, ubiquitination, and the accumulation of cytoplasmic vesicles around the periphery of the inclusions. The striking morphological and biochemical similarities between LBs and the intracellular accumulations in this model suggest that fibrillar seeds may have a fundamental role in the initial formation of LBs and other disease-associated filamentous inclusions. Also, the accumulation of assembly-competent α-Syn nucleation seeds may be an important rate-limiting factor for LB formation. Although the precise series of events leading to inclusion formation remain unclear, the data presented herein indicate α-Syn recruitment depends on the presence of an amyloidogenic sequence. Together with the observation that the majority of α-Syn within inclusions is endogenous, these findings suggest that endogenous α-Syn recruitment to fibrillar α-Syn seeds underlies the formation of these inclusions in the present cell culture system, and it is believed that similar processes lead to the formation and growth of LBs in PD and related synucleinopathies.

The absolute number of nucleation sites introduced into individual cells in the model has not been determined, although the biochemical data suggest that the amount of protein transduced represents a minor fraction of the endogenous α-Syn pool. Thus, small quantities of misfolded and fibrillar α-Syn may be sufficient to seed aggregation in the context of long-lived postmitotic cells such as neurons. However, little is known regarding how fibrillar nuclei initially arise in neurons and glia in vivo. Misfolded α-Syn could arise in a cell-autonomous manner via increased synthesis as seen in individuals with α-Syn gene amplification (Singleton et al., 2003, Science. 302:841) or by mutations that accelerate α-Syn misfolding itself (e.g., the familial A53T mutation) (Polymeropoulos et al., 1997, Science. 276:2045-2047). Generation of rapidly aggregating C-terminally truncated α-Syn species, as reported in PD brains (Li et al., 2005, Proc Natl Acad Sci USA 102:2162-2167), may also contribute to this process. Likewise, impairment of α-Syn degradation pathways or insults that alter the degradation or function of α-Syn could result in the accumulation of a critical mass of seeds. Indeed, the results presented herein indicate that, even in rapidly dividing cells, α-Syn fibrils remain longer in the intracellular space compared with soluble species, which may further promote its ability to recruit and convert endogenous α-Syn.

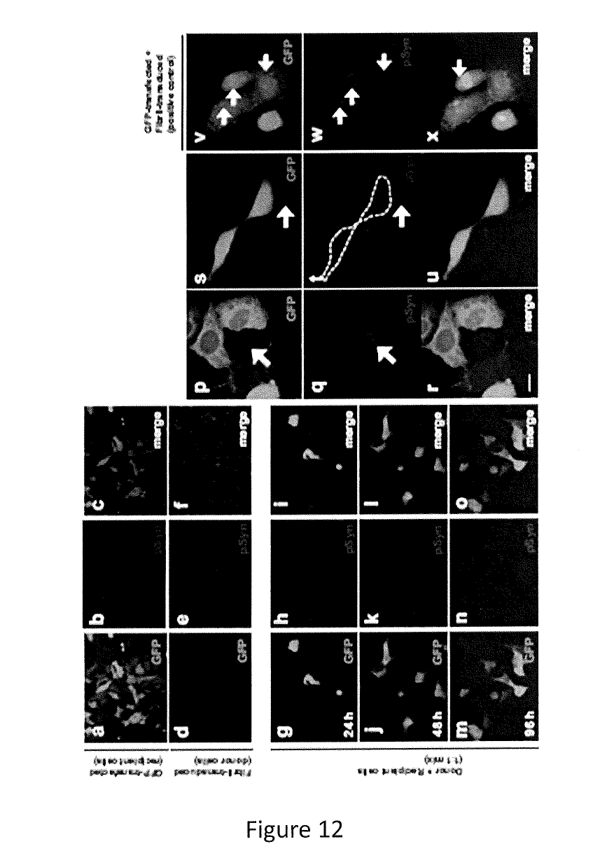

Another possibility is that α-Syn seeds enter from neighboring cells or the extracellular space as suggested by recent studies demonstrating that both neuronal and normeuronal cells participate in the release and uptake of soluble α-Syn species (Desplats et al., 2009, Proc Natl Acad Sci USA. 106:13010-13015). Supporting this notion, data from autopsied PD brains suggest that LBs appear in a progressive temporospatial pattern between closely connected regions of the nervous system (Braak et al., 2009, Adv Anat Embryol Cell Biol. 201:1-119). Also, recent studies indicate that embryonic dopaminergic neurons grafted into PD patients develop α-Syn inclusions, suggesting that pathology is conferred by proximity to pathological tissue (Kordower et al., 2008, Nat. Med. 14:504-506). However, there remains unclear whether released α-Syn or some other agent is responsible for the development of α-Syn deposits in the transplanted cells. Significantly, detection of inclusion formation in α-Syn overexpressing cells was not observed, even when cocultured in direct contact with cells already containing prominent inclusions (FIG. 12). The results presented herein, together with the observation that α-Syn pathology within grafts is seen after extended periods, suggest that transmission of misfolded seeds is a rare event.

The capacity for exogenously introduced amyloids to seed intracellular aggregation has also been recently reported for two neurodegenerative disease-related proteins. In contrast to the results with α-Syn, fibrils comprised of either tau (Frost et al., 2009, J Biol. Chem. 284:3546-3551) or polyglutamine-expanded proteins (Ren et al., 2009, Nat Cell Biol. 11:219-U232) appear to be actively taken into cells, including neurons, without reagent-mediated transduction. Significantly, when injected into the brains of transgenic mice expressing WT tau, which do not otherwise develop tau lesions, tissue homogenates containing misfolded mutant tau induce conformational changes and tau neuropathology, even in areas beyond the injection site (Clavaguera et al., 2009, Nat Cell Biol. 11:909-913). It is not clear why α-Syn fibrils were not efficiently introduced into cells in the absence of transduction reagent, although it is likely that not all fibrils are internalized in a similar manner. For example, it has been suggested that tau aggregates undergo endocytosis (Frost et al., 2009, J Biol. Chem. 284:3546-3551), whereas polyglutamine fibrils are internalized via an endosome-independent process (Ren et al., 2009, Nat Cell Biol. 11:219-U232). It is also possible that different cell types employ different mechanisms of fibril internalization.

The results presented herein focused on molecular processes that occur once α-Syn fibrils gain entry into cells. The results presented herein support the view that the sequence of events leading up to LB formation can be recapitulated in cultured cells. Within all cell types examined in this study, the vast majority of inclusions occupied a juxtanuclear position, although they failed to colocalize with any classically defined compartment. However, inclusions consistently colocalized with ubiquitin and multiple chaperones, which strongly suggests that they elicit the misfolded protein response and protein degradation pathways. Also, the α-Syn inclusions disrupt Golgi integrity, indicating that insoluble α-Syn inclusions are not benign. Further characterization of these intracellular changes should uncover how α-Syn inclusions influence key cellular processes. Intriguingly, although some previous studies suggest a possible link between α-Syn phosphorylation and cytotoxicity, the results presented herein show that inclusion formation does not require this modification. Nonetheless, the extent of α-Syn hyperphosphorylation observed in the model of the present invention and human LBs suggests that it may be an important postaggregation event.

The results presented herein, coupled with other recent results (Ren et al., 2009, Nat Cell Biol. 11:219-U232; Frost et al., 2009, J Biol. Chem. 284:3546-3551; Desplats et al., 2009, Proc Natl Acad Sci USA. 106:13010-13015; Clavaguera et al., 2009, Nat Cell Biol. 11:909-913), strongly suggest that several different amyloid fibrils can act as potent catalysts for the conversion of soluble proteins into amyloid fibrils, and highlight the importance of developing agents that prevent the formation of nucleating cores (or block further aggregate growth from existing seed structures) as a therapeutic strategy for the treatment of patients with neurodegenerative protein misfolding diseases. To this end, the model of the present invention provides significant insights into the events that regulate the formation of intracellular α-Syn inclusions and represents an invaluable tool for further elucidating the pathological mechanisms underlying this major family of diseases.

### Example 2

### Tau Aggregation

Filamentous aggregates comprised of misfolded proteins are neuropathological signatures of common sporadic and hereditary neurodegenerative diseases, α-Synuclein (α-syn) aggregates within neurons, known as Lewy bodies (LBs) and Lewy neurites (LNs), are the hallmark lesions of Parkinson's disease (PD). In addition to PD, many other neurodegenerative diseases also develop α-syn inclusions and they are collectively known as synucleinopathies. Similarly, fibrillar aggregates of tau protein are found as intraneuronal inclusions known as neurofibrillary tangles (NFTs) in Alzheimer's disease and a number of related “tauopathies”, including Pick's disease, supranuclear palsy and certain Frontotemporal lobar degenerative diseases. Previous studies have documented that filamentous α-syn and tau inclusions are linked to the onset/progression of synucleinopathies and tauopathies, and this is re-enforced by studies which demonstrated that transgenic mice engineered to express human mutant α-syn or mutant tau develop neuronal pathologies that recapitulate their human counterparts.

Purified recombinant human α-syn and tau readily assemble into amyloid fibrils in vitro under defined conditions but, thus far, there has been a paucity of robust mammalian cell-based models of α-syn or tau aggregation where the majority of the cells develop amyloid fibrils and inclusions. The results presented herein demonstrate a successful α-syn aggregation model in which preformed α-syn fibrils are introduced into cells that stably overexpress human α-syn. The intracellular fibrils serve as a nidus for the recruitment and association of endogenous α-syn, with the latter being phosphorylated and ubiquitinated as in LBs.

The following experiments were designed to develop a comparable tau aggregation model using similar methodology as that used in the α-syn cellular model discussed elsewhere herein. These cell-based models are extremely valuable in identifying molecules that modify intracellular pathways of α-syn and tau aggregation and degradation.

The approach used in generating intracellular tau inclusions is similar to that used for α-syn PFF as discussed elsewhere herein. To, generate tagged and untagged tau proteins, tau cDNAs are cloned into a pRK172 expression vector and expressed in E. coli. Recombinant tau was produced and purified as described in Li et al., 2006 Biochemistry 45 15692-15701). Tau fibrillization reactions to generate PFFs are performed as described (Crowe et al., 2009 Biochemistry 48: 7732-7745; Crowe et al., 2007 Biochemical and Biophysical Research Communications 358: 1-6) and the PFF integrity is monitored by amyloid binding dyes (e.g. ThT), sedimentation analyses and negatively stained electron microscopy (EM). To generate QBI293 stable clones expressing tau, the Lenti-X Tet-on Advanced inducible expression system (Clonetech) was utilized where transgene expression is tightly regulated. This is important because tau binds to and stabilizes MTs, making it difficult to achieve high levels of stable tau expression in QBI293 and other non-neuronal cells due to tau-mediated excessive MT stabilization which impedes cell division. To distinguish between tau PFFs and endogenous tau, cells stably expressing untagged T40 (for transduction using K18-myc PFFs) or Flag-tag T40 (for transduction with T40-myc PFFs) were generated. Tau expression in stable cell lines is induced using doxycycline, and PFFs are transduced using cationic-liposomal protein transduction reagent (Bioporter, Sigma) the next day, and the transduced cells were analyzed two days later. Transduced cells are extracted with 1% Triton-X 100 buffer to eliminate all soluble tau such that insoluble recruited tau is visualized using indirect immunofluorescence (IFs) with any of a variety of anti-tau antibodies to phosphorylation-dependent and -independent epitopes spanning the entire tau molecule. The use of different epitope tags allows for the ability to distinguish exogenous tau PFFs from endogenously expressed tau. A similar strategy can also be applied to primary cultures of neurons from tau transgenic mice that express human tau (Yoshiyama et al., 2007 Neuron 53: 337-351), except no transduction reagent is used and PFFs are added directly to the medium surrounding the neuronal cultures.

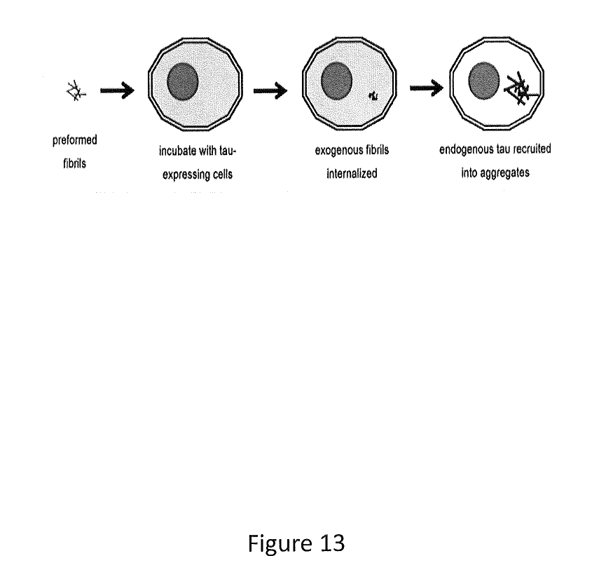

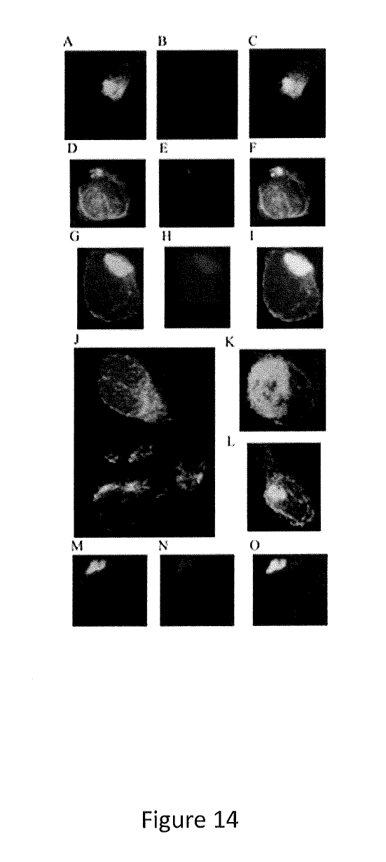

The cell culture models of tau inclusion formation of the present invention are based on the results presented elsewhere herein regarding α-syn. Briefly, pre-formed tau fibrils (PFFs), which have been produced from purified recombinant tau (Crowe et al., 2009, Biochemistry 48: 7732-7745) and have undergone sonication, are added to the medium surrounding cultured cells (see FIG. 13). Upon incubation, the cells internalize the PFF preparation and this exogenous misfolded tau serves to recruit endogenous tau expressed by the cells, resulting in aggregates with features that resemble NFTs and neuropil threads. As with the α-syn model, the efficiency of internalization of the PFF preparation can be facilitated by the use of a transduction reagent. The results presented herein demonstrate that this seeding phenomenon works with several cell types, including cell-lines such QBI293 cells or primary neuron cultures. As shown in FIG. 14, the addition of PFFs formed from full-length tau (T40) or truncated form of tau containing only the microtubule-binding domains (K18), with each containing a myc-tag to facilitate immunostaining, results in tau inclusion formation in QBI293 cells that express endogenous human tau.

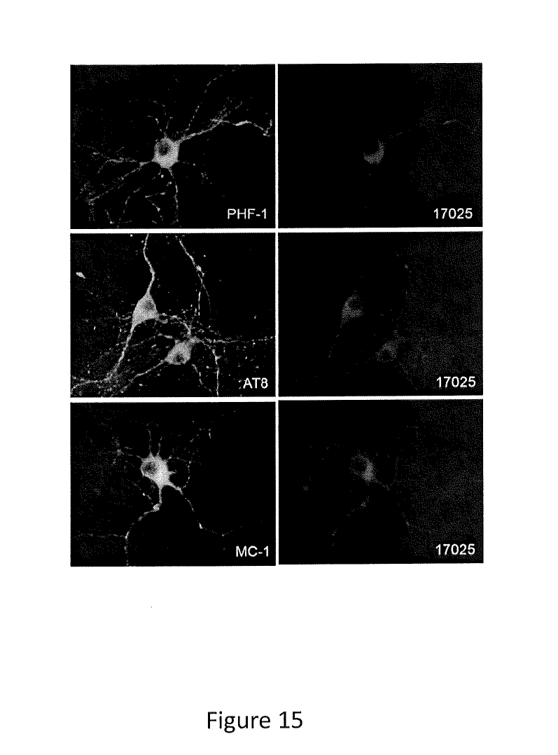

Moreover, similar tau inclusions can be induced in primary hippocampal neurons from Tg mice that express human tau with the P301 S mutation that causes FTDP-17 (PS19 mice) (Yoshiyama et al., 2007 Neuron 53: 33 7-351). This is demonstrated in FIG. 15, where hippocampal neuron cultures from PS19 embryos are treated with tau PFFs at day 6 in culture, followed by immunostaining 12 days later after permeabilization and fixing using a method in which only insoluble tau remains in the cells. As demonstrated in FIG. 15, the cell soma and processes are extensively stained by 17025 antibody that recognizes total tau. Moreover, the insoluble tau aggregates are phosphorylated, as evidenced by staining with the PHF-1 and AT8 antibodies that bind tau epitopes pS202/pT205 and pS396/pS404, respectively. Finally, the tau inclusions are stained with the MC-1 antibody (Jicha et al., 1997 Journal of Neuroscience Research 48: 128-132) that recognizes a misfolded epitope of tau. Thus, the inclusions that are formed in QBI293 cells and primary neurons have verisimilitude to NFTs found within the brains of those with a tauopathy. These cellular models thus provide a valuable research tool that can be exploited to identify molecules that affect either the formation or degradation of neuropathological tau aggregates.

### Example 3

### Elucidating the Pathogenesis of Neurofibrillary Tangles in a Cell Model with Tau Aggregation

Tau, a highly soluble microtubule (MT) associated protein, promotes the assembly and stabilization of MTs in healthy neurons, but forms hyperphosphorylated neurofibrillary tangles (NFTs) in Alzheimer's disease (AD) and related tauopathies. The lack of a cell model that develops robust NFTs is a major impediment in defining the underlying disease mechanisms in AD. The results presented herein demonstrate a cell model recapitulating key features of tauopathies via liposome-mediated delivery of preformed tau fibrils (tau PFFs) into tau-expressing cells. Significantly, internalization of minute quantity of PFFs can recruit endogenous tau into detergent-insoluble aggregates, which was greatly enhanced by the P301L tau mutation. Sequestration of soluble tau by PFF-induced tau aggregates attenuated MT over-stabilization in tau over-expressing cells, supporting the loss-of-function toxicity of NFTs. Intriguingly, spontaneous uptake of tau PFFs inducing substantial aggregation occurred through adsorptive endocytosis potentiated by WGA, demonstrating cellular transmissibility of tau pathology. Thus, the cell-based tauopathy model of the invention provides mechanistic insights into NFT pathogenesis and a system for identifying tau-based therapeutics.

The materials and methods employed in these experiments are now described.

### Materials and Methods

Reagents and Antibodies

BioPORTER® Quikease™ Protein Delivery Kit for fibril transduction, wheat germ agglutinin (WGA) and N-acetyl glucosamine (GlcNAc) were purchased from Sigma. Antibodies used in this study are listed and described in Table 4.

TABLE 4

| Antibody | Antigen | Host species | Dilution | Source |
|---|---|---|---|---|
| 17025 | Human | rabbit | 1:1000 (WB, ICC) | Ishihara et al., |
| | recombinant tau | polyclonal | | 1999 |
| PHF-1 | p-Tau | mouse | 1:1000 (WB, immuno- | Otvos et al., |
| | (phosphorylated | monoclonal | EM); 1:2000 (ICC) | 1994 |
| | at Ser 396 and | | | |
| | 404) | | | |
| AT8 | p-Tau | mouse | 1:500 (ICC) | Innogenetics |
| | (phosphorylated | monoclonal | | |
| | at Ser 202) | | | |
| MC-1 | Tau in the | mouse | 1:500 (ICC) | Gift from Dr. |
| | pathological | monoclonal | | Peter Davies |
| | conformation | | | Lab |
| Tau 5 | Tau (210-230aa) | mouse | 5 ug/ml as capture | Gift from Dr. |
| | | monoclonal | antibody in tau ELISA; | Lester Binder |
| | | | 6 ug for | Lab |
| | | | immunoprecipitating tau | |
| | | | from ~100 ug protein | |
| biotinylated | Tau (194-198aa) | mouse | 125 ng/ml as reporting | Pierce (Thermo |
| BT2 | | monoclonal | antibody in tau ELISA | Scientific) |
| | | | together with HT7 | |
| biotinylated | Tau (159-163aa) | mouse | 125 ng/ml as reporting | Pierce (Thermo |
| HT7 | | monoclonal | antibody in tau ELISA | Scientific) |
| | | | together with BT2 | |
| T46 | Tau (404-441aa) | mouse | 11 ug for | Kosik et al., |
| | | monoclonal | immunoprecipitating tau | 1988 |
| | | | from ~100 ug protein | |
| myc 9E10 | myc tag | mouse | 1:1000 (WB, ICC) | Developmental |
| | | monoclonal | | Studies |
| | | | | Hybridoma |
| | | | | Bank (DSHB) |
| Anti-c-myc | c-myc tag | rabbit | 1:5000 (regular ICC); | Sigma |
| | | polyclonal | 1:1000 (live staining) | |
| GAPDH (6C5) | Glyceraldehyde- | mouse | 1:3000 (WB) | Advanced |
| | 3phosphate | monoclonal | | Immunochemical |
| | dehydrogenase | | | |
| 12G10 | tubulin | mouse | 10 ug/ml as capture | DSHB |
| | | monoclonal | antibody in Ace-tub | |
| | | | ELISA | |
| Acetylated | Acetylated- | mouse | 1:4000 (ELISA); 1:1000 | Sigma |
| tubulin | tubulin | monoclonal | (ICC) | |
| (6-11 B-1) | | | | |

Recombinant Tau Purification and In Vitro Fibrillization

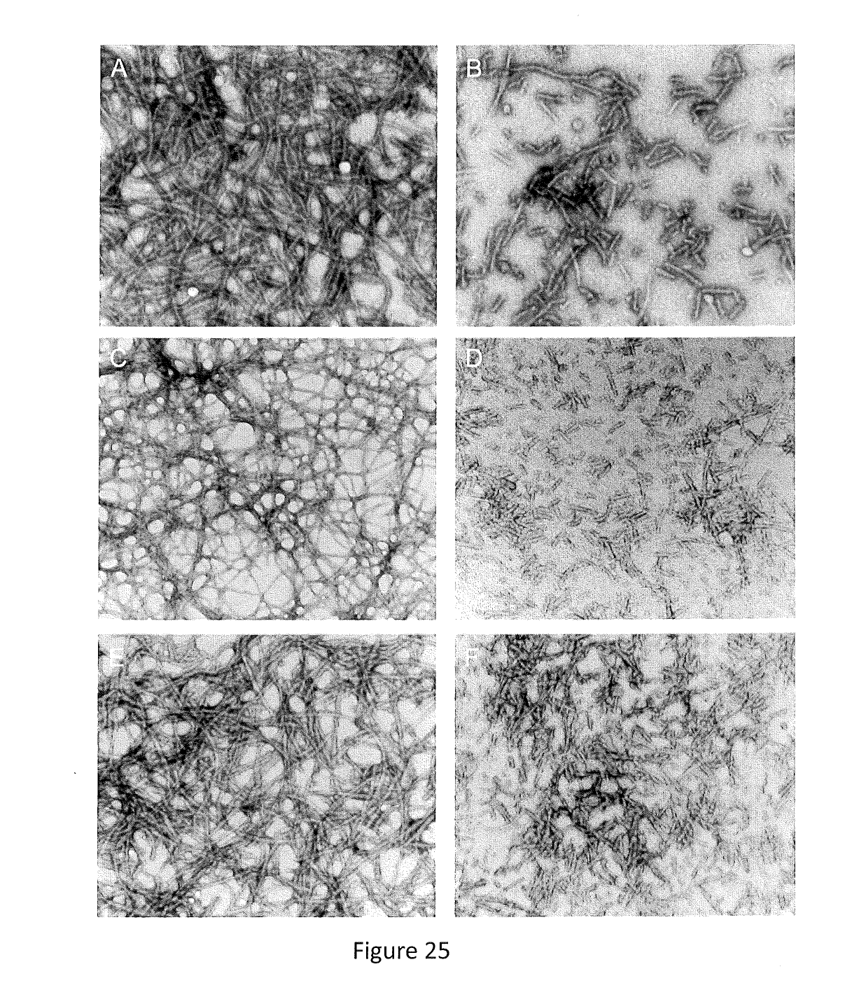

The cDNAs coding for (1) the longest isoform of tau (2N4R) with a myc tag at the 3′ end (3′myc-T40), (2) truncated tau containing only 4 MT-binding repeats with a myc tag at the 5′ end (5′myc-K18) and (3) 5′myc-K18 containing P301L mutation (5′myc-K18/P301L) were cloned into NdeI/EcoRI sites in pRK172 bacterial expression vector. Each protein was expressed in BL21 (DE3) RIL cells and purified by cationic exchange using a Fast Protein Liquid Chromatography (FPLC) column as previously described (Li, et al., 2006, Biochemistry. 45:15692-15701). In vitro fibrillization was conducted by mixing 40 μM recombinant tau with 40 μM low-molecular-weight heparin and 2 mM DTT in 100 mM sodium acetate buffer (pH 7.0). Except 3′myc-T40, which was agitated at 1,000 rpm for 5 days, the other proteins were incubated at 37° C. without shaking for 2-3 days. Successful fibrillization was confirmed using a ThT fluorescence assay, sedimentation test and negative stain electron microscopy (FIG. 25) as previously described (Crowe, et al., 2009, Biochemistry. 48:7732-7745). Before being used for transduction on cells, fibrillization mixtures were centrifuged at 100,000 g for 30 min at 4° C. The resulting pellet was resuspended in equal volume of 100 mM sodium acetate buffer (pH 7.0) without heparin and DTT, and frozen as single-use aliquots at −80° C.

Cell Culture and Fibril Transduction

QBI-293 cells (QBiogene) were grown in full media (DMEM, 10% FBS) supplemented with penicillin/streptomycin and L-glutamine. For fibril transduction, cells were plated at 400,000 cells/well in a 6-well tissue culture plate one day before transient transfection with WT or mutant tau in pcDNA5/TO or pcDNA3.1 (+) using FuGENE® 6 (Roche) as per manufacturer's instructions at 2 g DNA per well. About 16 hr after transfection, resuspended fibril aliquots were diluted to 10 M using 100 mM sodium acetate buffer (pH 7.0) and sonicated with 20-30 pulses. A volume of 80 μL of sonicated fibrils was added to one tube of BioPORTER reagent, gently vortexed for 5 sec and allowed to stand at room temperature (RT) for 5-10 min. During the formation of PFF/reagent complex, cells were washed once with 2 mL OptiMEM (Invitrogen) and placed on 500 μL OptiMEM before the fibril-reagent complex was diluted with 420 μL OptiMEM and added to cells. For experiments performed on cells plated on 12-well plates, all reagents were reduced by half. Six to seven hr after the addition of fibrils, cells were placed on starvation medium (DMEM, 0.5% FBS, penicillin/streptomycin, L-glutamine) to reduce cell division, 24 hr after which they were washed once with versene/EDTA, incubated with 0.05% trypsin/EDTA for 10 min at 37° C. to remove the majority of membrane-associated fibrils and replated onto poly-D-lysine-coated glass coverslips for immunostaining. Fixing was performed one night after replating.

For fibril-alone transduction without BioPORTER reagent, cells were placed on starvation medium right before the addition of fibrils. Fibrils were processed as described above and the same amount was used as in reagent-mediated transduction. Cells were trypsinized and replated either 4 hr or 30 hr after the initial addition of fibrils. For experiments using WGA, different doses of WGA were added to starvation medium together with PFFs. To inhibit endocytosis of WGA, 0.1 M GlcNAc was incubated with medium containing PFFs and 10 g/ml WGA for 1 hr at 37° C. before being added to cells which were pre-incubated in 0.1M GlcNAc-containing medium for 1 hr. Cells were placed on WGA-free starvation medium 5-6 hr after fibril addition and fixed after overnight incubation to minimize possible toxicity resulted from WGA treatment.

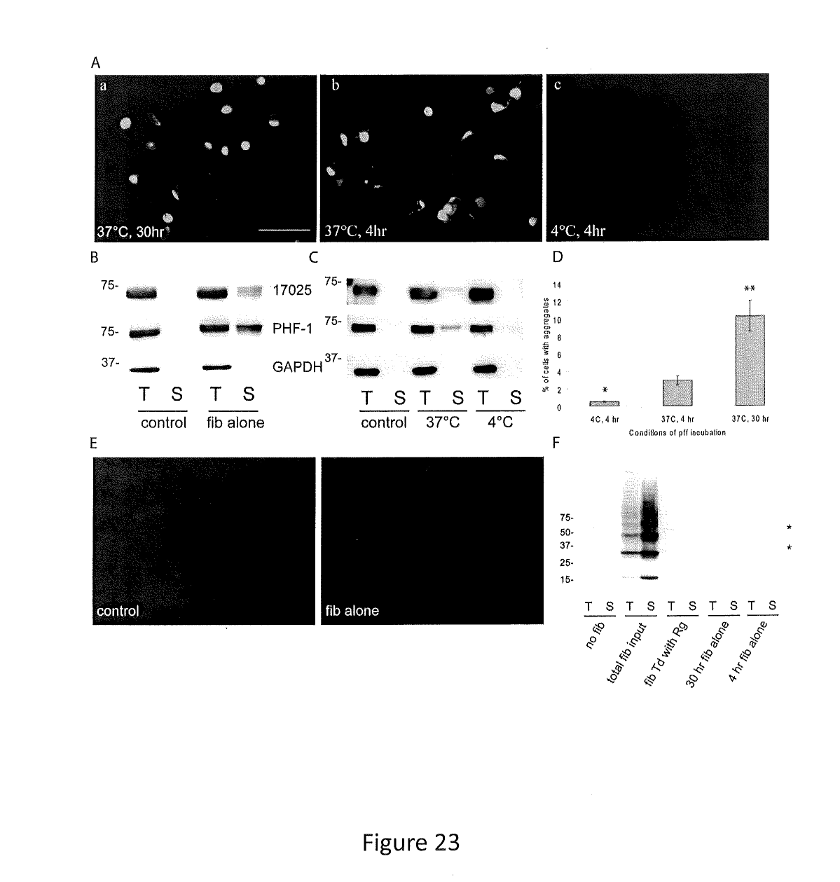

No replating was done for biochemical analysis except for experiments attempting to quantify cell-associated PFFs (FIG. 23F) and 4 hr fibril-alone transduction experiments (FIG. 23C) in which cells were transferred to new 6-well plates after the incubation period.

Immunocytochemistry

Immunocytochemistry was performed 48 hr after the initial addition of fibrils unless indicated otherwise. Cells were fixed in 4% paraformaldehyde (PFA) for 10 min and permeabilized with 0.1% Triton-X100 for 15 min, or fixed with 4% PFA containing 1% Triton-X100 for 15 min to remove soluble proteins. For visualization of microtubules, cells were fixed with 0.3% glutaraldehyde in PEM buffer (80 mM PIPES, pH6.8, 5 mM EGTA, 1 mM MgCl2) for 10 min, extracted with 1% Triton-X100 for 15 min before being quenched with 10 mg/ml sodium borohydride for 7 min followed by 0.1 M glycine for 20 min. After blocking with 3% BSA and 3% FBS for at least 1 hr at RT, cells were incubated with specific primary antibodies (see Table 4) for 2 hr at RT or overnight at 4° C. followed by staining with appropriate Alexa fluor 594- or 488-conjugated secondary antibodies (goat anti-mouse/rabbit/mouse IgG1/mouse IgG2b) for 1-3 hr at RT. DAPI was added to PBS wash to label cell nuclei. For ThS staining, cells were incubated with 0.005% ThS for 8 min and differentiated with 70% ethanol 4 times, 5 min each.

Two-stage immunostaining was employed to distinguish between membrane-associated and truly internalized PFFs. Live cells were incubated with rabbit polycloncal anti-myc Ab diluted in medium containing 25 mM HEPES for 1 hr at 4° C. After 3 washes with cold medium, cells were fixed with 4% PFA and permeabilized with 0.1% Triton-X100. Following blocking, cells were incubated with monoclonal anti-myc Ab 9E10 for 1-2 hr at RT. Rabbit myc Ab is expected to bind to cell membrane-associated PFFs only, where 9E10 would bind to both membrane-associated and intracellular PFFs. Therefore, truly internalized PFFs should only be recognized by 9E10 but not rabbit myc Ab.

Epifluorescence images were acquired using an Olympus BX 51 microscope (B&B Microscopes) equipped with a digital camera DP71 (Olympus) and DP manager (Olympus).

Sequential Extraction and Western Blot

Cells were washed once with PBS before being scraped into Triton lysis buffer (1% Triton-X100 in 50 mM Tris, 150 mM NaCl, pH 7.6) containing protease and phosphatase inhibitors. Following sonication, lysates were centrifuged at 100,000 g for 30 min at 4° C. Supernatants were kept as “Triton fraction”, while pellets were washed once in Triton lysis buffer, resuspended and sonicated in SDS lysis buffer (1% SDS in 50 mM Tris, 150 mM NaCl, pH 7.6). After centrifugation at 100,000 g for 30 min at 22° C., supernatants were saved as “SDS fraction”. Equal proportions of Triton and SDS fractions were resolved on 10% SDS polyacrylamide gels, transferred to nitrocellulose membranes, and blocked in 5% milk in TBS before probing with specific Abs (see Table 4). Protein-Free block (ThermoFisher) was used for PHF-1 blots.

Pulse Chase

Reagent-mediated transduction was performed as described above.

One day after transduction, cells were placed on methionine-free medium for 15 min and then pulsed with methionine-free medium containing [³⁵S]-methionine (150 μCi for each well on 12-well plate) for 20 min, after which cells were switched into complete medium containing 5 mM cold methionine and chased for different durations. During harvest, cells were washed once in HBSS and sequentially extracted using 1% Triton-X followed by 1% SDS lysis buffer. Tau was immunoprecipitated from cell lysates diluted in RIPA using a combination of mAbs T46 and Tau 5.

Electron Microscopy (EM)

To visualize PFFs made in vitro, 6 μL unsonicated or sonicated fibrils was placed on 300 meshed Formvar/carbon film-coated copper grids (Electron Microscopy Sciences) for 3 min, washed twice with 50 mM Tris buffer (pH 7.6) for 5 min each, stained with 10-15 drops of 2% uranyl acetate, and visualized with Jeol 1010 transmission electron microscope (Peabody).

Immuno-EM of reagent alone treated cells and fibril-transduced cells were performed as described previously (Luk, et al., 2009, Proc. Natl. Acad. Sci. U.S.A. 106:20051-20056) using PHF-1 as the primary antibody.

Sandwich ELISA for Quantifying Ac-Tub and Tau in Cell Lysates

Levels of acetylated tubulin (Ac-tub) and tau in cell lysates were quantified using a sandwich ELISA in 384-well plate format. For Ac-tub ELISA, tubulin mAb 12G10 was used as the capture antibody, and horseradish peroxidase (HRP)-conjugated Ac-tub mAb was used as the reporting antibody. Standard curves were generated using serially diluted brain lysates from wt mouse. Cell lysates (18 g) diluted in EC buffer (0.02 M sodium phosphate buffer pH7.0, 2 mM EDTA, 0.4M NaCl, 0.2% BSA, 0.05% CHAPS, 0.4% Blockace, 0.05% NaN3) were loaded in each well and incubated overnight at 4° C. On the following day, after 4 hr incubation with reporting antibody at room temperature, trimethy benzidine (TMB) peroxidase substrate solution was added and the plate was read using SpectraMax at 450 nm absorbance. For quantifying tau in Triton and SDS fractions, cell lysates from sequential extractions were diluted 1,000-foldin EC buffer. Serially diluted recombinant human T40 was used to generate standard curves. Diluted samples (30 μL) were loaded into each well of the plate coated with capture antibody Tau 5. After overnight incubation at 4° C., captured proteins were reported using biotin-labeled BT2 and HT7 antibodies which were left to incubate overnight at 4° C. On the next day, following 1 hr incubation with HRP-conjugated streptavidin at 25° C., the plate was developed using TMB peroxidase substrate solution.

Quantifications and Statistical Analysis

For quantifying the percentage of cells developing tau aggregates with different combinations of endogenous tau and PFFs, 10-20 random-field images were taken at 40× magnification for each combination and at least two independent experiments were performed. For quantification of MT-bundling and PFF-induced tau aggregation in the absence of bioPORTER reagent under various conditions (i.e. different temperatures, with or without WGA/GlcNAc), 10 random-field images were taken at 20× magnification for each condition in each experiment and 3-4 independent experiments were performed. Two-tailed paired student's t-test was used for all the comparisons in the study and differences with p values less than 0.05 were considered significant.

The results of the experiments are now described.

### Intracellular Tangle-Like Aggregates can be Induced by Preformed Fibrils (Pffs)

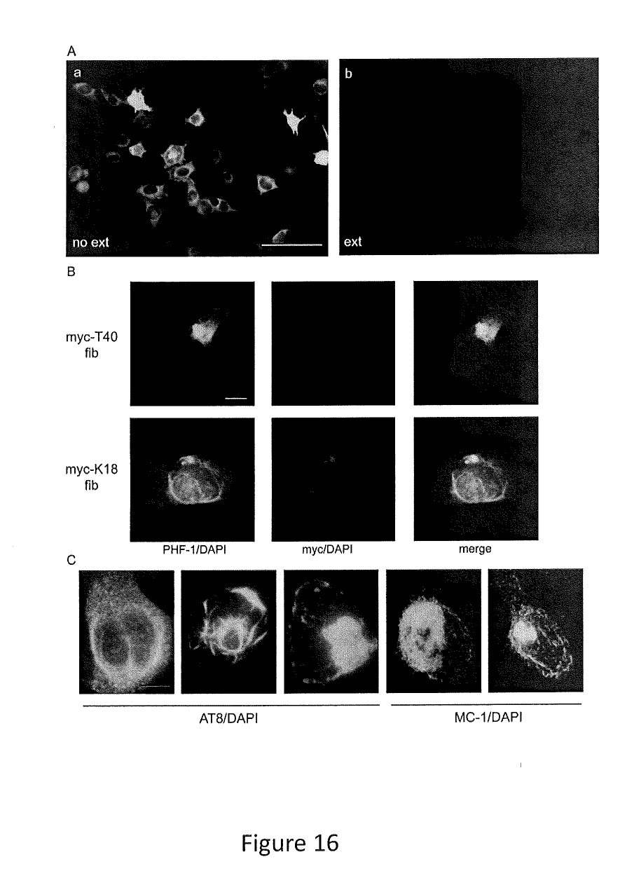

To explore the capability of tau PFFs to seed intracellular fibrillization of soluble tau, QBI-293 cells were transiently transfected with wild type T40 (wt-T40), the longest human tau isoform (2N4R), and transduced with PFFs generated from myc-tagged full-length human tau (myc-T40) or truncated tau containing only the 4 MT-binding repeats (myc-K18) (FIG. 25) using BioPORTER protein delivery reagent. Unlike wt-T40-expressing cells treated with transduction reagent alone, in which phosphorylated tau recognized by monoclonal antibody (mAb) PHF-1 (pSer396/404) was completely soluble and extracted by 1% Triton-X100 during fixation (FIG. 16A), cells transduced with either myc-T40 or myc-K18 tau PFFs showed accumulations of Triton-insoluble fibrillar phospho-tau aggregates (FIG. 16B). These induced aggregates were also detected by anti-phospho-tau mAb AT8 (pSer202/pThr205) and conformation-dependent mAb MC-1 (FIG. 16C) which specifically recognizes tau in a pathological conformation (Jicha, et al., 1997, J. Neurosci. Res. 48:128-132). Interestingly, PFF-induced aggregates exhibited a variety of morphologies, ranging from widely distributed short fibrils, skein-like accumulations, to localized dense NFT-like inclusions and sometimes a mixture of phenotypes (FIG. 16C).

Significantly, double-labeling immunofluorescence in wt-T40-expressing cells using anti-myc antibody to detect myc-K18 and myc-T40 pffs, and PHF-1 to detect phosphorylated T40, showed that anti-myc only labeled a fraction of the fibrils detected by PHF1, thus supporting a “seeding” and recruitment mechanism of tau aggregate formation (FIG. 16B). Moreover, phosphorylated tau was non-existent or rare in non-transfected cells transduced with myc-T40 PFFs, suggesting a lack of phosphorylation of internalized full-length fibrils (data not shown). Furthermore, since epitopes recognized by these three tau specific mAbs (PHF-1, AT8 and MC-1) are absent from myc-K18, their immunoreactivities in myc-K18 fibril transduced cells can be entirely attributed to endogenously expressed wt-T40. Thus, exogenously supplied tau PFFs do not constitute the insoluble phospho-tau species that were detect in wt-T40 cells treated with PFFs. Finally, accumulation of insoluble tau aggregates failed to occur when PFFs were transduced into cells expressing fibrillization-incompetent tau with a K311D mutation, further confirming the de novo fibrillization of soluble tau in wt-T40-expressing cells.

### P301L Mutation Enhances Seeded Recruitment of Endogenous Tau

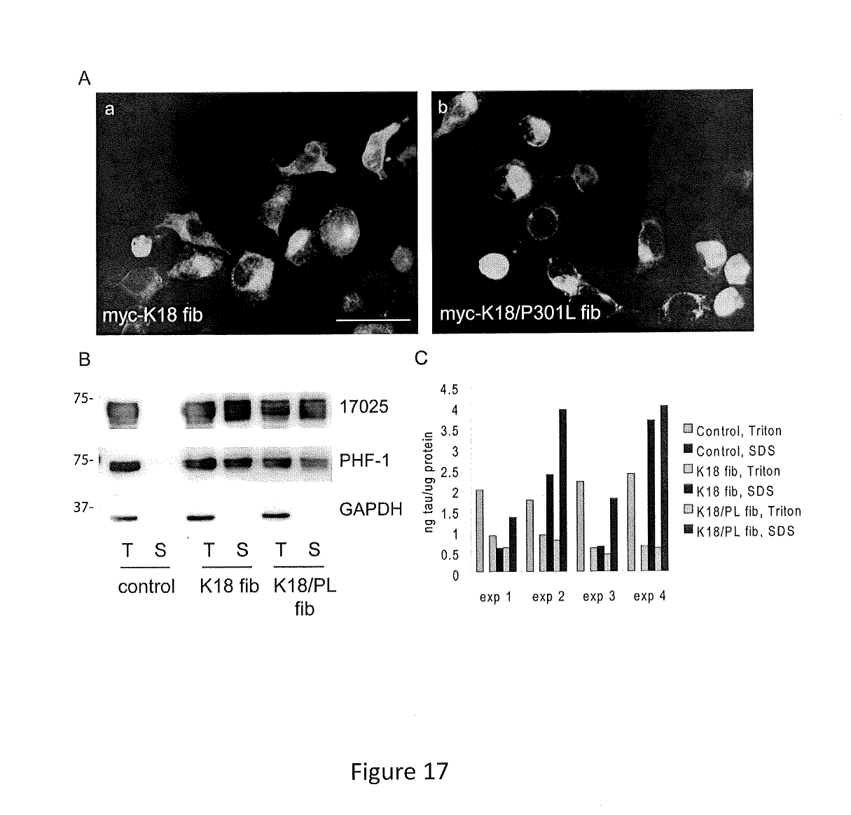

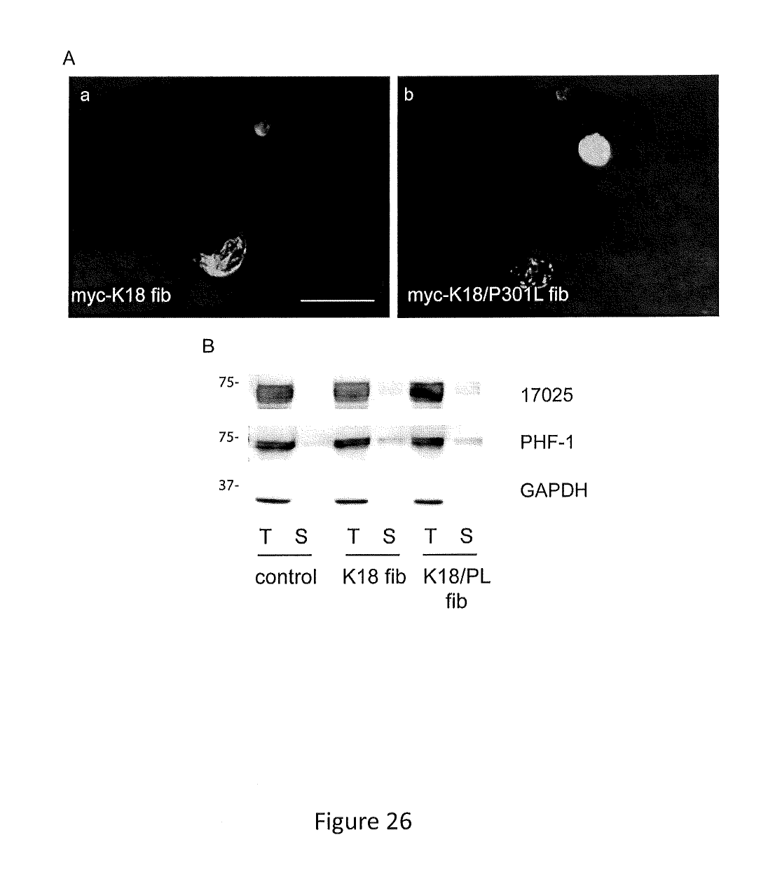

To assess the efficacy of recruitment by pffs of 4R tau, with or without N-terminal inserts, as well as disease-associated mutant tau, fibril transduction was performed on QBI-293 cells transiently transfected with human T43 (0N4R), T40/AK280 (FTDP-17 mutation in the MT-binding domain), T40/P301L (FTDP-17 mutation in the MT-binding domain), and T40/R406W (FTDP-17 mutation outside the MT-binding domain). Quantification of the percentage of aggregate-bearing cells induced by these various combinations revealed a similar extent of aggregation in T43- and T40-transfected cells upon myc-K18 fibril transduction (about 8% of cells had aggregates with either treatment), suggesting the number of N-terminal inserts does not affect fibrillization (Table 5). Among the dominantly inherited tau mutations in FTDP-17 studied here, T40 with the P301L mutation demonstrated the highest propensity of PFF-induced aggregation. Specifically, transduction of myc-K18 PFFs and myc-K18 PFFs with P301L mutation (myc-K18/P301L fibrils) led to about 20% and 35% of cells bearing aggregates, respectively (Table 5), corresponding to −40% and—70% of the transfected cells assuming 50% transfection efficiency (FIG. 17A). Although myc-K18/P301L fibrils appeared to be better “seeds” for T40/P301L recruitment as monitored by number of aggregate-bearing cells, they were comparable to, or even slightly less efficient than, myc-K18 fibrils in nucleating the fibrillization of wt-T40 (Table 5). Enhanced aggregation of T40/P301L as compared to wt-T40 corresponded to significantly more accumulation of Triton-insoluble tau as shown on immunoblots (compare FIG. 17B with FIG. 26B).

Table 5: Percentage of Aggregate-Bearing Cells with Specific Combinations of Endogenous Tau Transfection (Tf) and Fibril Transduction

TABLE 5
Percentage of aggregate-bearing cells with specific combinations
of endogenous tau transfection (Tf) and fibril transduction

| | Tf | | | | |
|---|---|---|---|---|---|
| | wtT40 | T43 | T40/ | T40/ | T40/ |
| Fibrils | (2N4R) | (0N4R) | P301L | ΔK280 | R406W |
| 5′myc-K18 | ~8% | ~8% | ~20% | ~5% | ~6% |
| 5′myc-K18/P301L | ~5% | NT^{a} | ~35% | NT^{a} | NT^{a} |

^{a}not tested

Quantification by tau sandwich ELISA indicated that myc-K18/P301L PFFs always resulted in higher amounts of Triton-insoluble T40/P301L tau than myc-K18 PFFs, confirming a more efficient recruitment of soluble tau with the combination of myc-K18/P301L PFFs and T40/P301L-expressing cells (FIG. 17C). Interestingly, both myc-K18 and myc-K18/P301L PFF transduction on T40/P301L-expressing cells invariably led to a reduction of Triton-soluble tau when compared with non-transduced cells, further supporting that the accumulation of insoluble tau is through sequestration/recruitment of soluble tau.

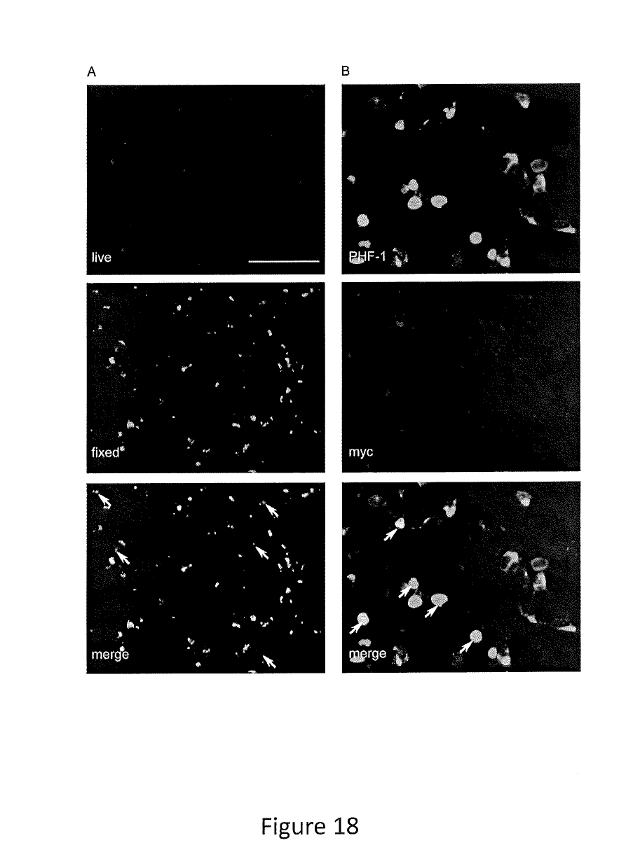

Intriguingly, live immunostaining followed by immunofluorescence after fixation and permeabilization (two-stage staining) revealed that the majority of PFFs associated with cells were merely membrane-associated despite extensive trypsinization before replating cells to coverslips, while only a small amount of PFFs were truly internalized (FIG. 18A). Moreover, double labeling using anti-myc antibody and PHF-1 in cells extracted with 1% Triton X-100 showed that less than half of the induced aggregates contained detectable colocalizing PFFs (FIG. 18B), suggesting that small amounts of PFF seeds are sufficient to induce robust endogenous tau fibrillization.

### PFF-Induced Tau Aggregates are Comprised of Filamentous NFT-Like Amyloid Structures

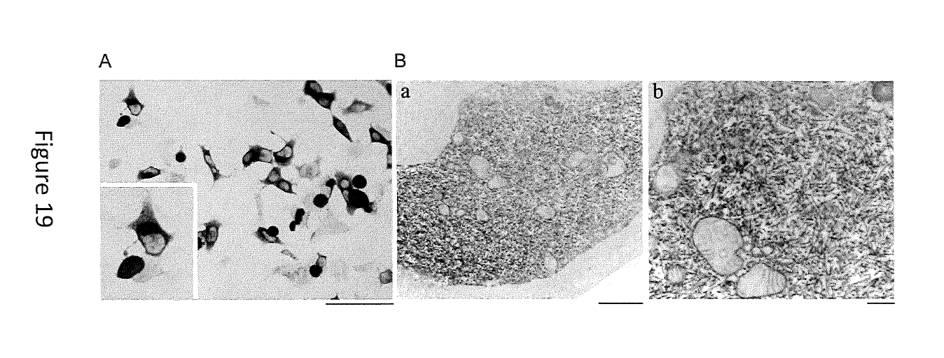

Immunocytochemical staining with PHF-1 in myc-K18/P301L PFF-transduced T40/P301L-expressing cells revealed inclusions that appeared remarkably similar to tau tangles in tauopathies morphologically. Interestingly, the formation of dense aggregates was often accompanied by clearance of diffuse cytoplasmic tau (FIG. 19A). Immuno-EM using PHF-1 revealed abundant cytoplasmic filaments in PFF-transduced cells (FIG. 19B), but not in the cells treated with transduction reagent alone. Moreover, diverse organelles, including mitochondria, lysosomes and other vesicular structures, were detected within and adjacent to PHF-1 positive filamentous structures (FIGS. 19B (a),(b)). The lack of diffuse PHF-1 staining in the cytoplasm outside of the PHF-1 inclusions when examined by both immunocytochemistry and immuno-EM further supports the concept of “seeding” and recruitment of soluble tau to form NFT-like structures in PFF-transduced cells.

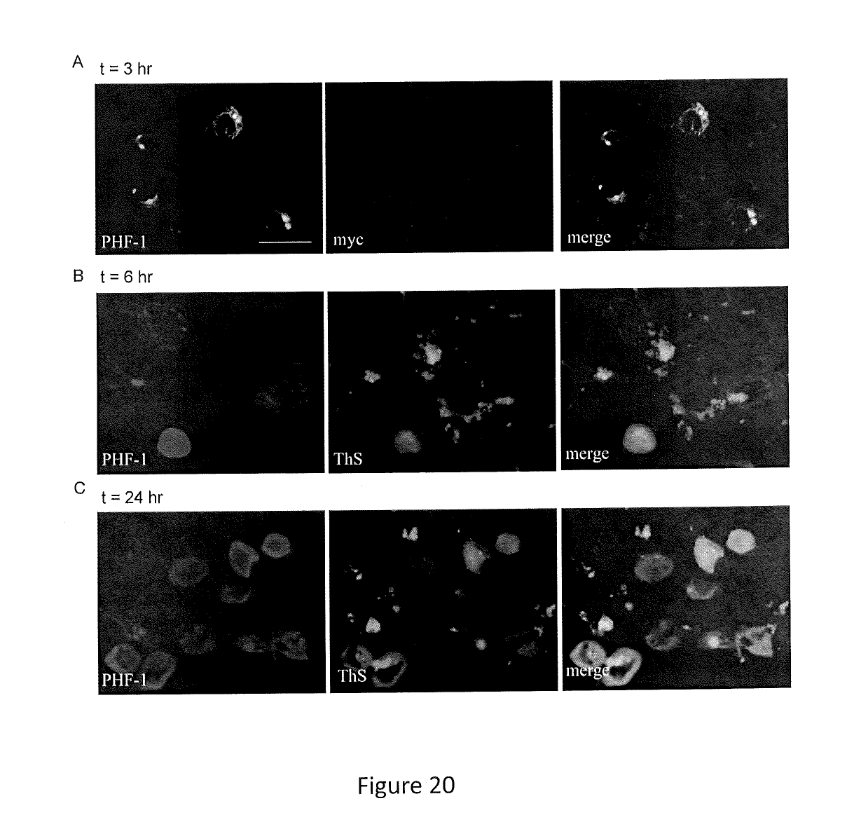

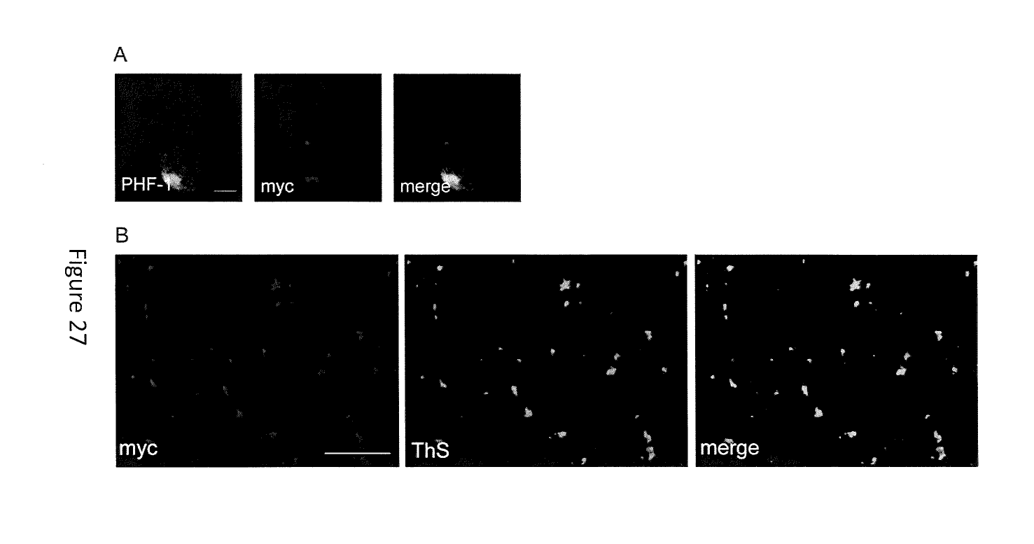

To examine the progression of PFF-induced endogenous tau aggregation, fixed cells at different time points after the addition of PFF/reagent complex were immunostained with PHF-1 and myc antibodies. At t=1 hr, there was little accumulation of Triton-insoluble tau, except for a few rare cells showing focal PHF-1 immunoreactivities with small but intensely labeled inclusions surrounded by slender, discrete fibrillar structures (FIGS. 20A and 27A). At t=3 hr, focal inclusions became more frequent and a small population of cells developed skein-like accumulations throughout the cytoplasm (FIG. 20A). It is noteworthy that the earliest focal accumulations were almost always colocalized with myc immunoreactivities, suggesting that aggregation first begins in cells containing more abundant fibril seeds which rapidly initiate the conversion of endogenous tau into insoluble aggregates through physical recruitment. At t=6 hr, insoluble tau was found in about 4% of total cells; although diffuse fibrillar accumulations remained the most common phenotype at this stage, some aggregates started showing signs of coalescence, and large compact inclusions appeared in a small subset of cells (FIG. 20B). Twenty-four hr after fibril transduction, about 25% of total cells were replete with Triton-insoluble tau, with a significant portion of cells bearing large, densely packed aggregates (FIG. 20C). Thioflavin S (ThS) was used to determine if PFF-induced tau aggregates form amyloid fibrils. Up to t=3 hr, ThS staining completely overlapped with myc immunoreactivities, indicating exclusive recognition of β-pleated sheet in myc-K18/P301L PFFs (FIG. 27B). ThS staining that colocalized with PHF-1 positive endogenous tau aggregates first emerged at t=6 hr, and became more abundant at t=24 hr (FIGS. 20B, 20C). Interestingly, only the large compact aggregates, but not diffuse skein-like inclusions, were recognized by ThS. Without wishing to be bound by any particular theory, it is believed that dispersed fibrillar aggregates represent early fibril species which eventually coalesce and mature into P3 sheet-rich inclusions.

Newly Synthesized Tau is Recruited Rapidly into Insoluble Tau Fibrils

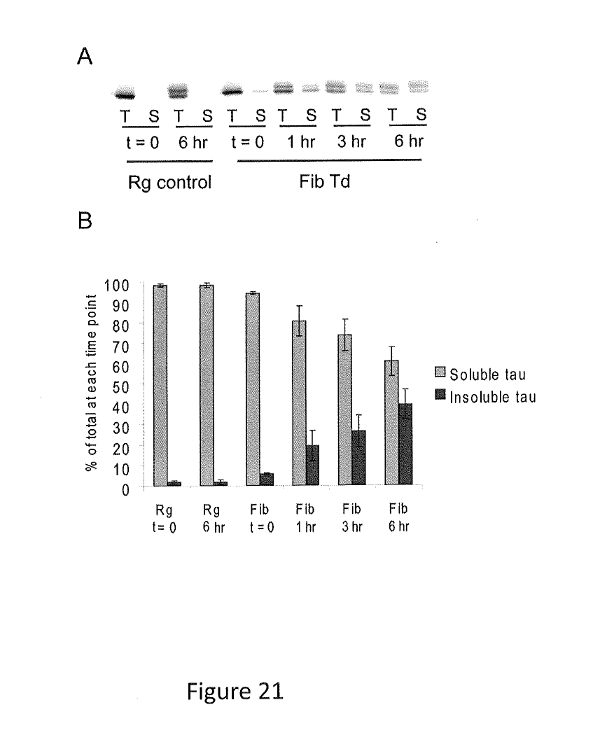

To directly monitor the recruitment of newly synthesized tau into insoluble aggregates, pulse-chase experiments on myc-K18/P301L PFF-transduced T40/P301L-expressing cells was performed. In cells treated with transduction reagent alone, [³⁵S]-radiolabeled tau remained soluble over a 6 hr chase period despite a time-dependent electrophoretic mobility shift indicative of phosphorylation of the newly synthesized tau. In contrast, an increasing proportion of radiolabeled T40/P301L tau emerged in the Triton-insoluble fraction of fibril-transduced cells, accompanied by more rapid reduction of soluble tau than in the control cells, demonstrating active recruitment of newly synthesized tau into an insoluble pool (FIG. 21). After a 1 hr chase, an appreciable amount of T40/P301L tau was already detected in the insoluble fraction, highlighting the rapid solubility change of tau in the presence of misfolded seeds. Moreover, the detection of slower migrating insoluble radiolabeled tau suggests continuous recruitment of soluble tau that is being modified by phosphorylation over time.

Reduced MT Stability is Associated with Tau Aggregation

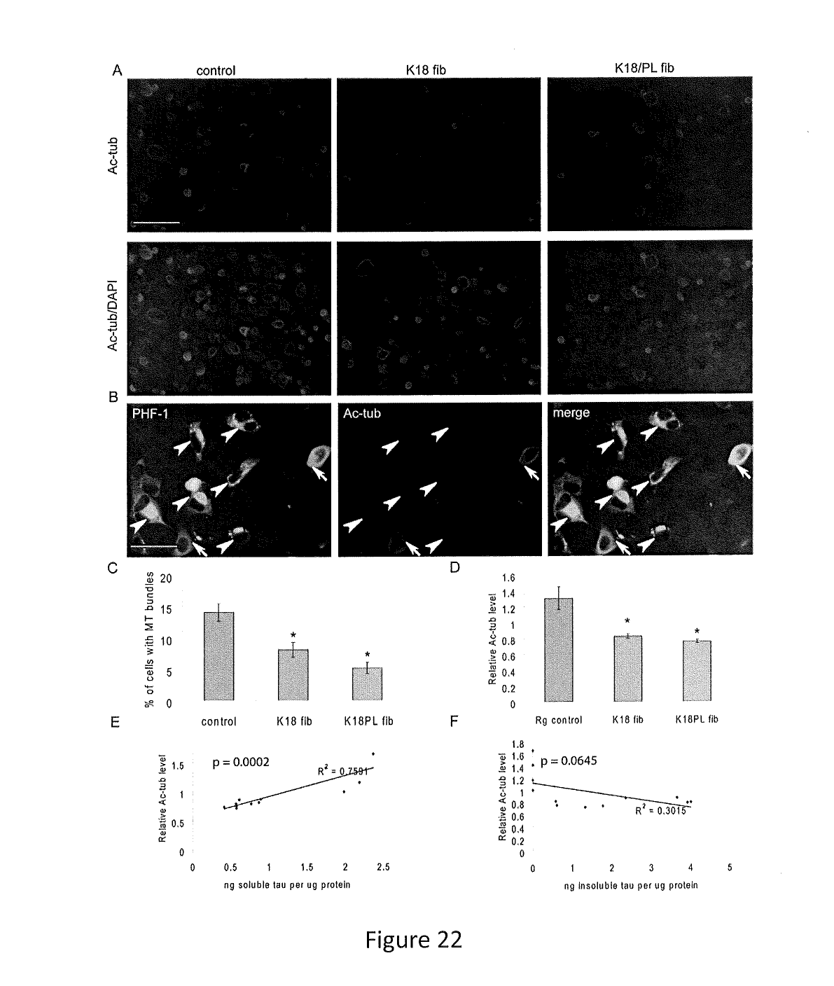

Consistent with previous studies in other cell lines, overexpression of tau in QBI-293 cells led to the formation of thick MT bundles due to excessive stabilization of MTs (Kanai, et al., 1989, J. Cell Biol. 109:1173-1184; Matsumura, et al., 1999, Am. J. Pathol. 154:1649-1656). Without wishing to be bound by any particular theory, it is believed that seeded recruitment of tau into insoluble fibrils may attenuate the over-stabilization of MTs by sequestering the soluble pool of tau. As expected, T40/P301L-expressing cells that were transduced with myc-K18 or myc-K18/P301L PFFs showed significantly less MT bundling as revealed by immunostaining of acetylated-tubulin (Ac-tub), a marker of stable MTs, as compared to those treated with transduction reagent alone (˜40% and ˜60% reduction in the percentage of cells with MT bundles by myc-K18 and myc-K18/P301L PFF transduction, respectively) (FIGS. 22A, 22C). The majority of cells with aggregates showed an absence of MT bundles and cells that retained bundles typically showed diffuse tau staining (FIG. 22B). Reduced Ac-tub levels associated with PFF-induced aggregation was further confirmed using an Ac-tub sandwich ELISA (FIG. 22D). In addition, there was a significant correlation between Ac-tub level and soluble tau, but not insoluble tau, suggesting that the loss of soluble tau rather than aggregation per se was responsible for the reduced MT stability (FIGS. 22E, 22F). This observation likely accounts for the occasional MT bundles observed in cells carrying aggregates.

### Spontaneous Fibril Uptake is Mediated by Endocytosis

To determine if tau PFFs can be taken up spontaneously in the absence of protein delivery reagent, T40/P301L-transfected cells were incubated with myc-K18/P301L PFFs for 30 hr, followed by trypsinization and incubation for an additional 18 hr. Aggregation of endogenous tau was detected in about 10% of total cells, accompanied by appreciable accumulation of Triton-insoluble tau detected on immunoblots (FIGS. 23A (a), 23B). Interestingly, exogenous PFFs were barely visible by immunostaining (FIG. 23E), suggesting that internalization of minute quantities of PFFs without transduction reagent is sufficient to induce recruitment of endogenous tau into fibrils. To investigate whether this uptake of tau PFFs occurs by endocytosis, T40/P301L-expressing cells were incubated with myc-K18/P301L PFFs for 4 hr at either 37° C. or 4° C.; the latter temperature blocks endocytosis. Since the amount of PFFs internalized by cells is too low to be easily detected by either immunostaining or immunoblotting (FIGS. 23E, 23F), Triton-insoluble endogenous tau aggregates was quantified as an indirect readout of PFF entry. PFF incubation at 37° C. for 4 hr prior to trypsinization and an additional 44 hr incubation resulted in 3% of total cells with tau inclusions, whereas 4 hr pff incubation at 4° C. followed by exactly the same treatments resulted in a dramatic and significant reduction in the percentage of tau aggregate-bearing cells (FIGS. 23A, 23C, 23D), suggesting PFF uptake is a temperature-dependent process. The small number of cells with tau aggregates (˜0.5% of total cells) detected after 4° C. PFF incubation could be due to the internalization of residual membrane-associated fibrils following trypsinization and additional incubation. Moreover, the difference in the extent of aggregation induced by 4 hr versus 30 hr incubation of PFFs (˜3% vs. ˜10% aggregate-bearing cells) indicated a time-dependent internalization of fibrils (FIG. 23D). These results suggest spontaneous uptake of PFFs probably occurs by endocytosis.

### Adsorptive Endocytosis Enhances Tau Pff Uptake

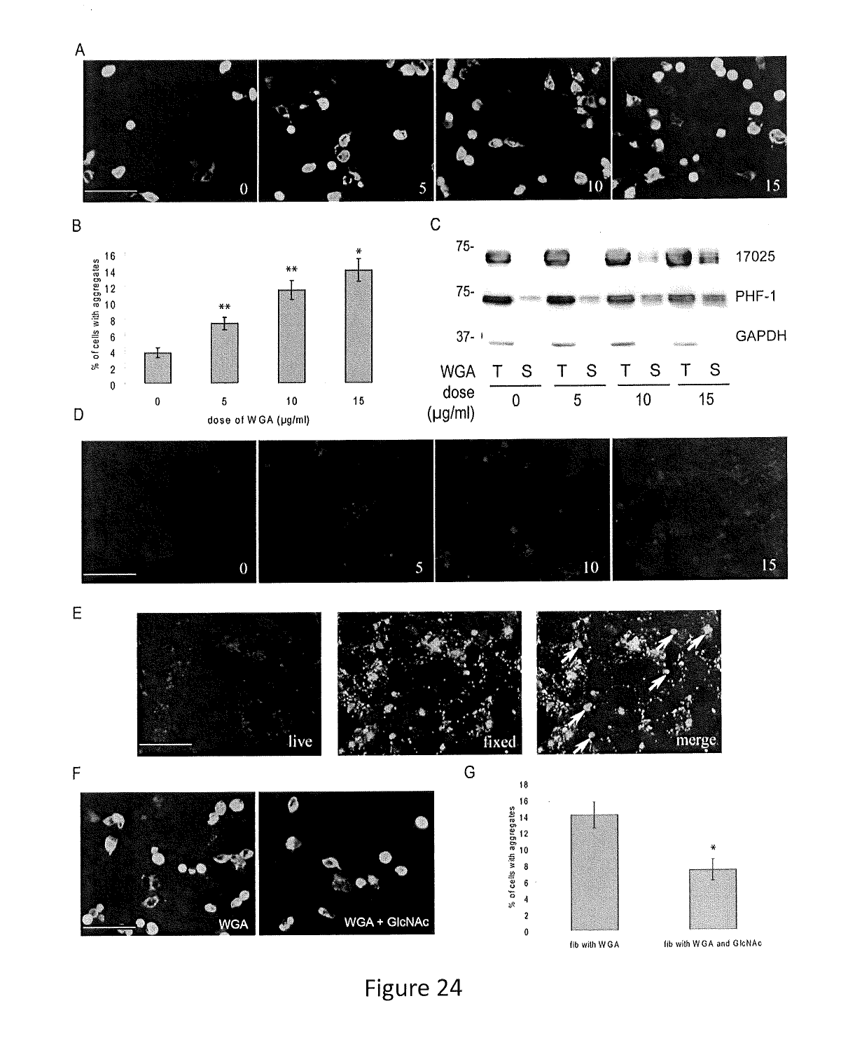

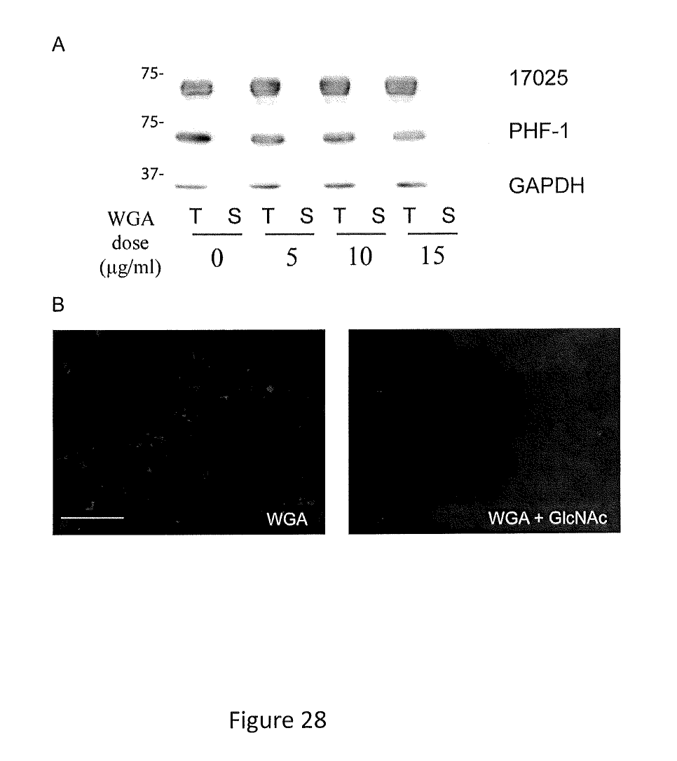

To further explore potential mechanisms of endocytosis-mediated PFF entry, cells were incubated with PFFs in the presence of WGA, a plant lectin that has high affinity for N-acetyl glucosamine (GlcNAc) and sialic acid on the plasma membrane and is readily internalized by cells via adsorptive endocytosis (Gonatas, et al., 1973, J. Cell Biol. 59:436-443; Broadwell, et al., 1988, Proc. Natl. Acad. Sci. U.S.A. 85:632-636). Apart from being a substrate of the endocytic pathway, WGA was also shown to potentiate the uptake of other proteins mediated by the same endocytic pathway (Banks, et al., 1997, Life Sci. 61:PL119-25; Banks, et al., 1998, J. Cell. Sci. 111 (4):533-540). Interestingly, it was found that WGA increased the prevalence of tau aggregate-bearing cells (FIGS. 24A, 24B) and the amount of Triton-insoluble tau in fibril transduced cells (FIG. 24C) in a dose-dependent manner, which paralleled increased cellular association with PFFs (FIG. 24D). Without WGA treatment, there was minimal fibril staining even without trypsinization. Increasing the dose of WGA not only led to progressively more abundant cell-associated PFFs, but two-stage staining revealed that a substantial percentage of these fibrils were indeed intracellular (FIG. 24E). Furthermore, the effects of WGA on PFF uptake and tau aggregation were significantly attenuated by adding 0.1 M GlcNAc to the medium, which reduces WGA-plasma membrane interactions through competition with GlcNAc on the plasma membrane (Gonatas, et al., 1973, J. Cell Biol. 59:436-443), suggesting that WGA-mediated induction of adsorptive endocytosis is necessary for increased PFF uptake and augmented tau aggregation (FIG. 28B and FIGS. 24F, 24G). On the other hand, up to 15 g/ml of WGA does not increase the expression level of tau, nor does it induce accumulation of insoluble tau in the absence of PFFs (FIG. 28A). Therefore, increased intracellular tau aggregate formation by WGA treatment is primarily due to enhanced internalization of PFFs. Taken together, spontaneous PFF entry seems to occur at least in part through adsorptive endocytosis which can be potentiated by WGA.

Elucidating Pathogenesis of NFTs in a Cell Model with Tau Aggregation

The results presented herein demonstrates that exogenously supplied tau PFF seeds can recruit soluble endogenous tau into insoluble fibrillar aggregates which bear resemblance to NFTs based on a variety of criteria, including immunofluorescence, amyloid dye binding, immuno-EM and biochemical analysis. Interestingly, a small quantity of internalized tau PFFs appears sufficient to recruit and sequester large amounts of endogenous tau into filamentous tau inclusions. Sequestration of soluble tau by PFF-induced aggregation significantly attenuates MT over-stabilization resulting from tau overexpression, supporting the concept that there is a reduction of soluble tau in neurodegenerative tauopathies. Intriguingly, substantial aggregation of soluble tau can also be induced by the spontaneous uptake of PFFs, which seems to occur via adsorptive endocytosis that is potentiated by the plant lectin WGA, demonstrating a cellular transmissibility of tau pathology.

Although the uptake and “seeding” capacity of preformed tau fibrils in cultured cells was reported recently (Frost, et al., 2009, J. Biol. Chem. 284:12845-12852; Nonaka et al., 2010, J Biol. Chem. 285(45):34885-98), the induction and formation of intracellular aggregates in the results presented herein are fundamentally different from these previous studies in several ways. First, the intracellular aggregates induced in the present model are morphologically different from previous studies and are comprised primarily of phosphorylated endogenous tau. Second, unlike previous models, detectable PFF seeds rarely coincide with the intracellular inclusions in the instant model; instead, the majority of aggregates that are composed of recruited endogenous tau greatly exceed the physical size of the seeds. Third, similar to tau tangles detected in AD and other tauopathies, the intracellular aggregates exhibit a diversity of morphologies, ranging from spatially distributed skein-like accumulations to large and densely packed aggregates, which probably represent different stages of aggregation. Fourth, the inclusions observed are clearly fibrillar in nature and mature into β-pleated sheet rich fibrils recognized by ThS. Fifth, the use of protein delivery reagent to augment introducing PFFs into cells provides better efficiency of tau PFF entry as compared to previous reports. In addition, unlike previous studies that expressed endogenous tau tagged with a fluorescent protein for recruitment by tau PFFs, the present system employs untagged tau which facilitates recruitment since the large fluorescent protein tag interferes with the conformational change required for efficient tau fibril assembly.

For the past 20 years, significant efforts have been devoted to produce cell-based models that develop tau aggregates similar to those observed in tauopathies but with little success. This is because tau is a highly soluble and naturally unfolded protein and therefore it has been notoriously difficult to make it aggregate in cultured cells. Strategies have been used in the past with minimal success to produce tau tangles in cell culture, including overexpression of aggregation-prone tau mutants (Vogelsberg-Ragaglia, et al., 2000, Mol. Biol. Cell. 11:4093-4104), overexpression of truncated tau that fibrillizes more readily than full-length tau (Khlistunova, et al., 2006, J. Biol. Chem. 281:1205-1214; Wang, et al., 2007, Proc. Natl. Acad. Sci. U.S.A. 104:10252-10257), addition of pre-aggregated amyloid-beta fibrils (Ferrari, et al., 2003, J. Biol. Chem. 278:40162-40168), treatment conditions that promote tau phosphorylation (Sato, et al., 2002, J. Biol. Chem. 277:42060-42065), and the addition of aromatic dyes such as Congo red to overcome the kinetic barrier of fibrillization (Bandyopadhyay et al., 2007, J. Biol. Chem. 282:16454-16464). In model of the present invention, however, tau aggregation could be rapidly induced within hours after the introduction of small quantities of misfolded tau PFFs which serve as templates to seed the recruitment of endogenous normal tau, resulting in the accumulation of large amounts of insoluble tau fibrils. Thus, such robust induction of authentic tangle-like accumulations suggests that a seeding-recruitment process is a highly plausible mechanism underlying NFT formation in vivo in the absence of tau overexpression. Conceivably, PFF-induced aggregation that occurs within hours/days in tau-overexpressing cultured cells could be prolonged into a pathological event that happens over years or even decades under physiological conditions, i.e. in human brains.

The results presented herein also demonstrate that the formation of intracellular tau tangles can be accelerated by FTDP-17 genetic mutations that were shown previously to increase the fibrillization propensity and/or reduce the MT-binding affinity of tau (Hasegawa, et al., 1998, FEBS Lett. 437:207-210; Hong, et al., 1998, Science. 282:1914-1917; Nacharaju et al. 1999, FEBS Lett. 447:195-199; von Bergen, et al., 2001, J. Biol. Chem. 276:48165-48174). Among the FTDP-17 associated mutations (AK280, P301L and R406W) tested in the present model, only P301L, the most aggressive disease causing mutation found in FTDP-17 patients, dramatically promoted aggregation. This correlates well with the rapid clinical onset and progression of neurodegenerative tauopathy observed in FTDP-17 patients (Spillantini, et al., 1998, Proc. Natl. Acad. Sci. U.S.A. 95:7737-7741; Mirra, et al., 1999, J. Neuropathol. Exp. Neurol. 58:335-345; van Swieten, et al., 1999, Ann. Neurol. 46:617-626) and transgenic mice harboring P301L mutant tau (Lewis, et al., 2000, Nat. Genet. 25:402-405; Gotz, et al., 2001, J. Biol. Chem. 276:529-534; review by Lee, et al., 2005, Biochim. Biophys. Acta. 1739:251-259). However, unlike a previous study that was unable to demonstrate recombinant P301L mutant tau seeding wt tau in a cell-free system (Aoyagi, et al., 2007, J. Biol. Chem. 282:20309-20318), the results presented herein demonstrate that wt and P301L mutant tau can cross-seed each other readily although fibril seeds composed of P301L mutant are slightly less efficient in recruiting wt-T40 than wt seeds. In addition, the difference in aggregation propensity between wt tau and P301L tau observed in the present cellular model may be further magnified in a physiological setting where tau is not over-expressed. This may explain the absence of insoluble wt tau deposition in FTDP-17 patients with P301L mutation (Miyasaka, et al., 2001, J. Neuropathol. Exp. Neurol. 60:872-884), where the overwhelmingly higher fibrillization propensity of P301L mutant tau could mask the much slower aggregation of wt tau that may not occur to an appreciable extent during a short disease course.

Despite lower MT-binding affinity of T40/P301L compared with wt-T40, QBI-293 cells transiently over-expressing T40/P301L mutant tau are still capable of MT bundling. Interestingly, PFF-induced aggregation resulted in significant attenuation of this phenotype. Reduced stable MTs is a well-established feature of tauopathy brains (Paula-Barbosa, et al., 1987, Brain Res. 417:139-142; Hempen, et al., 1996, J. Neuropathol. Exp. Neurol. 55:964-972; Boutte, et al., 2005, J. Alzheimers Dis. 8:1-6), but studies exploring the relationship between compromised MT stability and tau aggregation were based on postmortem brain examination and therefore are only correlative. The results presented herein is the first to demonstrate a direct causal link between tau aggregation and reduced MT stability, although it has been shown that MT stability is reduced following phosphatase treatment of cultured neurons which increases tau phosphorylation (Merrick, et al., 1997, J. Neurosci. 17:5726-5737). Thus, both tau aggregation and hyperphosphorylation could act synergistically to reduce MT stability. Significantly, a strong correlation between MT stability and the amount of soluble tau, but not insoluble tau, was observed suggesting depletion of soluble tau into fibrillar aggregates rather than aggregates themselves are responsible for a diminution of stable MTs. Although these observations were made in an artificial cell system with overexpression of tau, it is highly plausible that tangle formation in human brains, where the majority of tau is normally bound to MTs, could impair the integrity of MTs by a similar mechanism of soluble tau sequestration. Given the critical role of MTs in axonal transport and consequently neuronal survival, the results presented herein support the loss-of-function hypothesis of NFT toxicity. Consistent with this interpretation are studies demonstrating the beneficial effects of MT-stabilizing drugs in mouse models of tauopathy without direct modulation of tau aggregation (Zhang, et al., 2005, Proc. Natl. Acad. Sci. U.S.A. 102:227-231; Brunden, et al., 2010, J. Neurosci. 30:13861-13866).

Although the results presented herein did not address the origination of misfolded seeds in vivo, it was discovered that the amount of internalized PFFs required for robust aggregation to occur was quite small and barely detectable in the present system. This finding holds important implications for tauopathies: it is conceivable that a small amount of misfolded seeds of tau slowly accumulated over decades in post-mitotic neurons can eventually initiate a full-blown cascade of massive aggregation of soluble tau, rendering tangle formation a self-perpetuating event once initiated. The sonicated PFFs that were introduced into cells contain a heterogeneous mixture of fibrillar species with varying dimensions, and intracellular aggregates that formed without coincident detectable PFFs were likely seeded by species that were too small to be visualized by light microscopy.

Consistent with recent studies pointing to the possible transmissibility of disease-associated amyloid fibrils (reviewed by Aguzzi et al., 2009, Neuron. 64:783-790; Goedert, et al., 2010, Trends Neurosci. 33:317-325), it was found that preformed tau fibrils in the absence of protein delivery reagent can also induce formation of tangle-like accumulations in a substantial population of cells, albeit to a lower extent than in the presence of transduction reagent. This result provides further evidence of the “prion-like” properties of tau, and may explain the stereotypical propagation of tau pathology in AD brains (Braak, et al., 1991, Acta Neuropathol. 82:239-259) and the successful application of immunotherapy in ameliorating tau pathology in a mouse model (Asuni, et al., 2007, J. Neurosci. 27:9115-9129). Moreover, spontaneous uptake of tau fibrils appears to occur via endocytic pathways that are temperature- and time-dependent. The potentiation of tau inclusions formation by WGA treatment suggests that fibril entry is probably mediated, at least partly, by adsorptive endocytosis. This represents a potential mechanism through which aggregated tau released from dying/dead neurons or from extracellular ‘ghost tangles’ may gain access into surrounding healthy neurons where it nucleates fibrillization of soluble tau.

In summary, the results demonstrate a simple, and yet highly reproducible and robust, cell model of tau aggregation recapitulating key features of tauopathies. This system has been employed to gain important insights into the onset and progression of tau pathology. The present model not only provides an invaluable tool for studying the pathogenesis of tau aggregation, but also offers a potential system for identifying therapeutic strategies against neurodegenerative tauopathies, such as small molecules inhibiting fibrillization of tau or antibodies blocking the spontaneous cellular uptake of fibril seeds.

### Example 4

### Exogenous α-Synuclein Fibrils Induce Lewy Body Pathology Leading to Synaptic Dysfunction and Neuron Death

Inclusions composed of α-synuclein (α-syn), i.e., Lewy bodies (LBs) and Lewy neurites (LNs), define synucleinopathies including Parkinson's disease (PD) and dementia with Lewy bodies (DLB). As described herein, preformed fibrils generated from full-length and truncated recombinant α-syn enter primary neurons and promote recruitment of soluble endogenous α-syn into insoluble PD-like LBs and LNs. Remarkably, endogenous α-syn was sufficient for formation of these aggregates, and overexpression of wild-type or mutant α-syn was not required. LN-like pathology first developed in axons and propagated to form LB-like inclusions in perikarya. Accumulation of pathologic α-syn led to selective decreases in synaptic proteins, progressive impairments in neuronal excitability and connectivity, and, eventually, neuron death. Thus, data described herein contribute important insights into the etiology and pathogenesis of PD-like α-syn inclusions and their impact on neuronal functions, and they provide a model for discovering therapeutics targeting pathologic α-syn-mediated neurodegeneration.

Experiments were designed to evaluate whether α-syn pffs, formed from purified recombinant human WT α-syn (α-syn-hWT), recruit endogenous α-syn into pathologic, insoluble inclusions. As described herein, α-syn pffs are internalized and induce endogenous α-syn expressed in primary neurons to aggregate into inclusions resembling LBs and LNs in human PD brains. LN-like accumulations are initially detected in axons and α-syn pathology then propagates to the cell body where LB-like inclusions develop. Formation of these PD-like α-syn LNs and LBs causes selective reductions in synaptic proteins, and progressive impairments in neuronal network function and excitability that culminate in neuron death.

The materials and methods employed in these experiments are now described.

### Materials and Methods

Primary Neuronal Cultures

Primary neuronal cultures were prepared from E16-E18 C57BL/6 mouse brains (Charles River, Wilmington, Mass.) and α-syn −/− mice (Abeliovich et al., 2000, Neuron 25:239-252). All procedures were performed according to the NIH Guide for the Care and Use of Experimental Animals and were approved by the University of Pennsylvania Institutional Animal Care and Use Committee. Hippocampi were dissected, rinsed with Hank's Balanced Salt Solution (HBSS) and digested in HBSS containing papain (20 U/mL; Worthington Biochemical Corporation; Lakewood, N.J.) containing DNase (20 μg/mL; Worthington Biochemical Corporation; Lakewood, N.J.). Following rinses in plating media (10% fetal bovine serum, 1% penicillin/streptomycin, Glutamax, B27, Neurobasal media; Invitrogen, Carlsbad, Calif.) and HBSS, dissociated hippocampal neurons were plated in plating media onto poly-D-lysine (0.1 mg/mL, diluted in borate buffer, 0.1M, pH8.4) coated coverslips (Carolina Biological Supply, Burlington, N.C.) in 24 well trays at 20,000-40,000 cells/cm² or poly-D-lysine coated 60 mm dishes at 70-100,000 cells/cm2. The media was then changed to neuronal media (1% penicillin/streptomycin, Glutamax, B27, Neurobasal media). Most experiments were performed at 19 days in vitro (DIV).

Preparation and Fibril Transduction

Recombinant full-length and truncated α-syn with and without a C-terminal myc-tag, were purified as previously described (Giasson et al., 2001, J. Biol. Chem. 276:2380-2386). α-syn pffs were generated by incubating purified α-syn (5 mg/mL in PBS) at 37° C. with constant agitation for 5 days, followed by aliquoting and storage at 80° C. The presence of amyloid was confirmed using Thioflavin T fluorometry. Fibrils of α-syn synthetic NAC peptide (amino acids 61-95) (Biotechnology Resource Center, Yale University) were generated as described (Giasson et al., 2001, J. Biol. Chem. 276:2380-2386). Pffs were diluted in PBS at 0.1 mg/mL, sonicated several times, and diluted in neuronal media. For a 24-well tray, 1 μg/mL of pffs were added, and 5 μg/mL of α-syn pffs were added to a 60 cm dish. For WGA experiments, α-syn pffs were incubated with 1 μg/mL or 5 μg/mL WGA or preincubated for 1 hr (h) with 0.1 M GlcNAC followed by incubation with media containing α-syn pffs, GlcNAC, and WGA.

Indirect Immunofluorescence

Neurons were fixed with 4% paraformaldehyde in PBS containing 4% sucrose followed by permeabilization with 0.1% Triton X-100. To determine if the pathologic α-syn aggregates were detergent-insoluble, neurons were fixed with 4% paraformaldehyde, 4% sucrose and 1% Tx-100 (Luk et al., 2009, Proc. Natl. Acad. Sci. USA 106:20051-20056). Following fixation, neurons were blocked with 3% bovine serum albumin (BSA) in PBS, and incubated in primary antibodies (Table 6) followed by Alex fluor 594 or 488-conjugated secondary antibodies (anti-mouse, anti-rabbit, anti-IgG1 and anti-IgG2a; Invitrogen; Carlsbad, Calif.) diluted in blocking buffer. To assess cell viability, cells were incubated with ethidium homodimer (2 μM, Sigma Aldrich, St. Louis, Mo.) and Hoechst 33342 (1:1000, Invitrogen, Carlsbad, Calif.) and the percent overlap between both dyes was determined using Metamorph software (Molecular Devices, Sunnyvale, Calif.).

Two-stage immunofluorescence was performed as described previously (Guo and Lee, 2011, J. Biol. Chem. 286:15317-15331) to distinguish between extracellular and intracellular α-syn-hWT pffs using mABs LB509 and Syn204. For two-stage immunofluorescence, neurons were treated with 100 ng/mL α-syn-hWT pffs and immunofluorescence was performed 2 weeks later. Live cells were labeled with mAB Syn204 (IgG2a) which selectively recognizes human pffs, followed by fixation, permeabilization and labeling with human α-syn specific antibody, LB509 (IgG1). Neurons were incubated in Alexa fluor 594 anti-IgG2a and Alexa fluor 488 IgG1-specific secondary antibodies. Coverslips were mounted on to slides using Prolong gold antifade reagent (Invitrogen; Carlsbad, Calif.). Epifluorescent images were acquired using an Olympus BX 51 microscope equipped with a digital camera DP71 and DP manager (Olympus; Center Valley, Pa.). Confocal laser scanning images were captured with a Zeiss LSM10 microscope (Thornwood, N.Y.). Unless otherwise stated, all experiments were performed a minimum of 3-10 times.

TABLE 6
Primary antibodies used

| Antibody | Source/Reference |
|---|---|
| α-syn, mouse | Unpublished, polyclonal rabbit antibody |
| specific | generated against amino acids 115-125 of |
| | mouse α-syn |
| CSPα | Synaptic Systems, Goettingen Germany |
| Complexin | Synaptic Systems, Goettingen Germany |
| Dynamin I | Epitomics, Burlingame, CA |
| GAPDH | Millipore, Billerica, MA |
| GluRI | Santa Cruz Biotechnology, Santa Cruz, CA |
| 81A (p-α-syn) | Waxman et al., 2008, J Neuropathol Exp Neurol. |
| | 67: 402-16 |
| Syn202 (total syn) | Giasson, et al., 2000, J Neurosci Res. 59: 528-33 |
| Syn207 (β-syn) | Tu et al., 1998, Ann Neurol. 44: 415:22 |
| Syn204 (human | Giasson, et al., 2000, J Neurosci Res. 59: 528-33 |
| α-syn) | |
| Syn214 (α and β syn) | Giasson, et al., 2000, J Neurosci Res. 59: 528-33 |
| MAP2 | Unpublished, rabbit polyclonal raised to bovine |
| | MAP2. |
| Myc | Sigma, St. Louis MO, rabbit polyclonal |
| p-α-syn 6.1 | Unpublished, rabbit polyclonal generated against |
| | peptide CAYEMPSEEGYQ (underline denotes |
| | phosphorylated residue) |
| PSD95 | NeuroMab, University of California, Davis, CA |
| Snap25 | Synaptic Systems, Goettingen Germany |
| SNL1 (α-syn | Giasson, et al., 2000, J Neurosci Res. 59: 528-33 |
| C-terminus) | |
| SNL4 (α-syn | Giasson, et al., 2000, J Neurosci Res. 59: 528-33 |
| N-terminus) | |
| Synapsin I | Invitrogen, Carlsbad, CA |
| Synapsin II | Epitomics, Burlingame, CA |
| Synaptophysin | Millipore, Billerica, MA |
| Syntaxin I | Sigma, St. Louis MO, clone HPC1 |
| T49 (tau) | Kosik et al., 1988, Neuron. 1: 817-25 |
| Tyrosine Hydroxylase | Pelfreeze, Rogers, AR |
| Ubiquitin | Dako, Carpinteria, CA |

EM and Immuno-EM

Primary hippocampal neurons were seeded onto 25 mm Thermanox coverslips (Nunc) and fixed 14 days following addition of pffs. Neurons for transmission EM were fixed for 2 hours (h) in 2.5% glutaraldehyde in 0.1 M cacodylate buffer, pH 7.4, post-fixed for 1 h in 1% OsO4 and 1.5% potassium ferrocyanide in 0.05 M cacodylate buffer, dehydrated in ethanol and embedded in Epon. For immuno-EM, neurons were fixed for 4 h in periodate-lysine-paraformaldehyde, followed by rinses with 50 mM NH₄Cl in PBS. Neurons were permeabilized with 0.05% saponin in PBS with 2% fish gelatin (PBS-FG) and 0.05% thimerosal followed by incubation overnight at 4° C. in mAB 81A diluted in 0.0005% saponin in PBS-FG. Neurons were then incubated in biotinylated horse anti-mouse IgG (Vector Laboratories, Burlingame, Calif.) followed by Avidin-Biotin Complex Elite (Vector Laboratories, Burlingame, Calif.) and post-fixed for 15 min in 1.5% glutaraldehyde in 0.1 M cacodylate buffer with 5% sucrose. Following rinses in 50 mM Tris, pH 7.6 containing 7.5% sucrose and 0.05% thimerosal, neurons were developed in 0.2% DAB containing 10 mM imidazole with 0.01% H2O2 followed by post-fixation and embedding as for transmission EM. For immunogold labeling, neurons were fixed and incubated in mAB 81A as for HRP immuno-EM followed by incubation with goat anti-mouse IgG coupled to nanogold (Nanoprobes, Yaphank, N.Y.). The nanogold labeled neurons were post-fixed for 20 min, gold toned with 0.05% gold chloride, and post-fixed, dehydrated in ethanol and embedded in Epon. Sections were counterstained with uranyl acetate and lead citrate. EM images were collected using a Jeol 1010 electron microscope at the University of Pennsylvania's Biomedical Imaging Core.

Sequential Extraction and Immunoblot Analyses

Neurons were scraped into 1% Tx-100 in Tris-buffered saline (TBS) (50 mM Tris, 150 mM NaCl, pH 7.4) and protease and phosphatase inhibitor cocktail at 4° C. Lysates were sonicated and centrifuged at 100,000 3 g for 30 min. The pellet was washed and suspended in 2% SDS in TBS. Samples were separated by SDS-PAGE and immunoblotting was performed using primary antibodies described in Table 6. Following incubation in primary antibody, blots were incubated in horseradish peroxidase-conjugated secondary antibodies (Jackson Immunoresearch; West Grove, Pa.) and developed using enhanced chemiluminescence. Digital images were acquired using a Fuji Film Intelligent Darkbox II (Fuji Systems, Tokyo, Japan) and quantified using the Multi Gauge program (Fujifilm). All immunoblots were performed a minimum of 3-8 times. Data were analyzed by ANOVA with Dunnett's posthoc test.

Microfluidic Chambers

Microfluidic neuronal culture devices consisted of a prefabricated poly-dimethylsiloxane (PDMS) neuronal device attached to a glass coverslip (Taylor et al., 2005, Nat Methods 2:599-605). Devices patterned with 2 100 μm high somal compartments connected by a series of microgrooves (3 μm high, 10 μm wide, 150 μm long) were obtained from Xona Microfluidics (Temecula, Calif.). Glass coverslips (No. 1, 24×40 mm, Corning Inc.), were coated with poly-D-lysine (0.4 mg/mL in borate buffer), and affixed to neuronal devices as per the manufacturer's instructions. Assembled devices were pre-wetted with media and verified to ensure consistent flow in chambers and microchannels. A total of 10,000 freshly dissociated hippocampal neurons were loaded per somal compartment. After a 30 min attachment period, additional media was added to each compartment. A 50 μL difference in media volume was maintained between the two compartments to regulate the direction of flow via microgrooves. Neurons were used for experiments at >7 DIV. Media levels were maintained throughout experiments to prevent passive flow of pffs to the opposite compartment. α-syn 1-120-myc pffs (2 g) were added to the neuritic compartment (retrograde experiments) or the somal compartment (anterograde experiments) and were fixed 7-12 days later. The retrograde experiments were repeated 4 times and anterograde experiments repeated 3 times, each in triplicates.

Calcium Imaging and Network Analyses

Hippocampal neurons were plated on 35 mm MatTek dishes at a density of 300,000 cells/dish and treated with either PBS (control) or 5 g α-syn-hWT pffs for varying lengths of time. Thirty minutes prior to scheduled observation, neurons were loaded with the calcium-sensitive fluorescent dye, Fluo-4-AM (1 μM, Invitrogen, Carlsbad, Calif.) at room temperature. Immediately prior to imaging, neuronal cultures were rinsed in buffered calcium saline solution (CSS in mM: 126 NaCl, 5.4 KCl, 1 MgCl₂, 1.8 CaCl₂, 10 HEPES, 25 glucose, pH 7.4)) and placed on a Nikon TE2000-U microscope, equipped with a Yokogawa spinning disk confocal unit (CSU-10B, Solamere Technologies, Inc.), Coolsnap HQ2 CCD camera (Roper Scientific), and operated with MetaMorph software. Spontaneous calcium activity from ˜200 neurons in the field of view was recorded for 5 min, at 10 Hz acquisition speed for 5 min using a 488-nm excitation laser and Nikon 10×/0.45 Plan Apo objective. Excitatory tone in the network was determined using a modified approach (Breskin et al., 2006). Synchronous oscillations were forced via network disinhibition (bicuculline, 100 μM, Tocris) and by slowly adding increasing doses of the AMPAR antagonist, NBQX, until synchronous oscillations stopped. The final concentration of NBQX required to disintegrate the propagation of activity within the excitatory network relative to the published IC50 (47 nm, (Dev et al., 1996, J Neurochem 67:2609-2612)) was used as an indication of excitatory tone (Breskin et al., 2006, Phys Rev Lett 97(18):188102). At the end of an imaging experiment, NMDA (100 μM+10 M glycine) was added to discriminate neurons from glia.

Custom-coded MATLAB scripts were used to analyze the images. Individual neurons in the field of view were identified, based on their response to NMDA, and the mean fluorescence within each soma was computed across the image stack. After removing high-frequency noise (Pardo et al., 2006, Ultrasonics 44(Suppl 1):e1063-1067), a wavelet-based peak detection algorithm was applied to identify the timestamps of calcium transient onset (Nenadic and Burdick, 2005, IEEE Trans Biomed Eng 52:74-87). The timestamps were used to generate a raster plot, which provides a compact visual representation of the population activity over time. The level of coordinated activity, or synchronicity, was determined using synchronization cluster analysis (Bialonski and Lehnertz 2006, Phys Rev E Stat Nonlin Soft Matter Phys 74(5 Pt 1):051909; Li et al., 2010, Biophys J 98:1733-1741). Briefly, an equal-time correlation was used to estimate the correlation between all pairs of neurons and to produce a correlation matrix. Eigenvalue and eigenvector decomposition of this matrix yielded a global synchronization index, which ranges from 0 (random, non-coordinated activity) to 1 (perfectly synchronous activity). Functional connectivity was determined by using surrogate time-series to test for significant interactions between all pairs of neurons (Li et al., 2010, Biophys J 98:1733-1741). The average number of functional connections in the network was then determined by computing the expected value of the degree distribution. Data were analyzed by ANOVA with Tukey's posthoc test.

The results of the experiments are now described.

### Induction of Insoluble α-syn Aggregates in Primary Neurons by α-syn Pffs

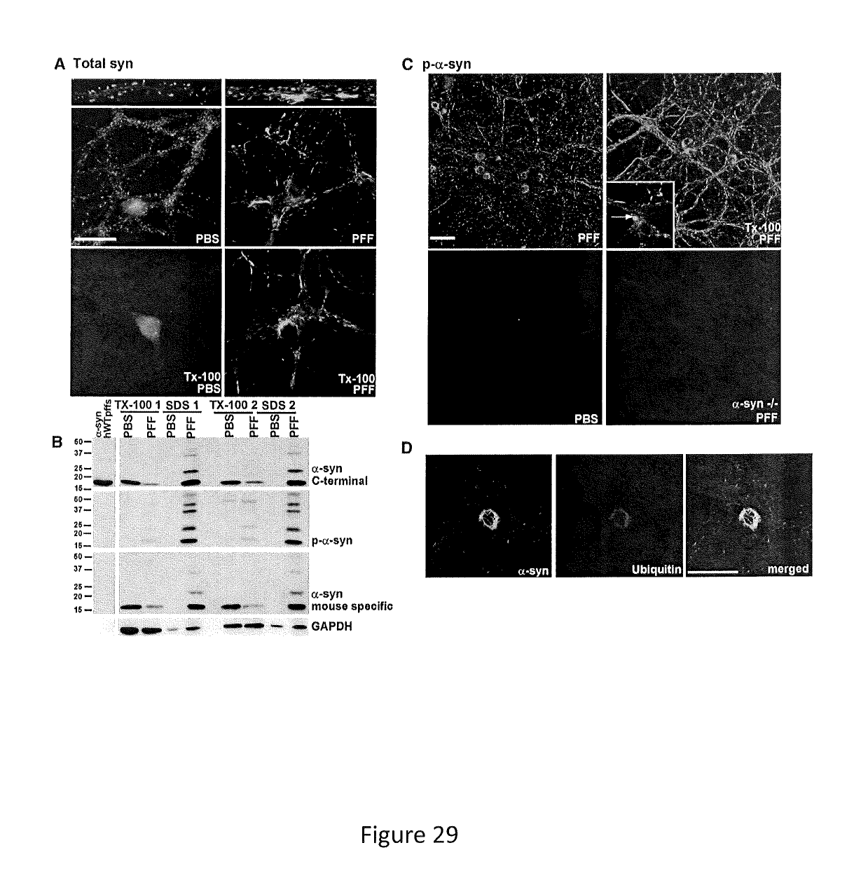

To determine whether exogenous human α-syn pffs can seed recruitment of endogenously expressed mouse α-syn into insoluble LB-like and LN-like fibrillar aggregates, α-syn pffs generated from full-length recombinant α-syn-hWT were added to primary hippocampal neurons derived from WT C57BL6 mice after culturing them for 5-6 days in vitro (DIV). These neurons were examined 2 weeks after the addition of α-syn-hWT pffs, when synapses are mature, and α-syn is normally localized to presynaptic terminals (Murphy et al., 2000, J. Neurosci. 20:3214-3220). In PBS-treated hippocampal neurons, endogenous mouse α-syn localized to presynaptic puncta as visualized using monoclonal antibody (mAB) Syn202, a pansynuclein antibody (Giasson et al., 2000, J. Neurosci. Res. 59:528-533) (FIG. 29A, top panels). In contrast, in α-syn-hWT pff-treated neurons, α-syn did not localize to the presynaptic terminal (FIG. 29A), but instead formed fibrillar LN-like inclusions. To determine whether the α-syn aggregates were detergent insoluble, PBS and α-synhWT pff-treated neurons were extracted with buffer containing 1% Triton X-100 (Tx-100) during fixation. Under such conditions, endogenous α-syn within neuronal processes in PBS-treated neurons was soluble in Tx-100, but cells incubated with α-synhWT pffs showed Tx-100-insoluble aggregates (FIG. 29A).

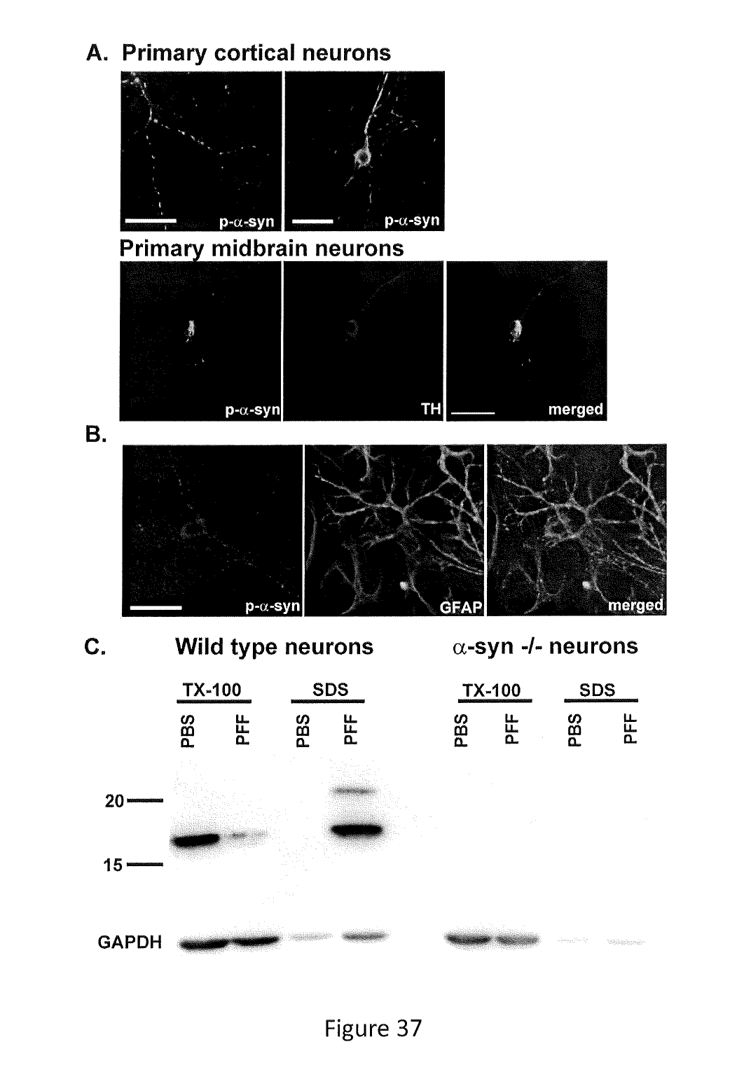

α-syn recruited into pathologic inclusions undergoes extensive phosphorylation at Ser129 (pSer129); thus antibodies against pSer129 selectively recognize α-syn pathology (Fujiwara et al., 2002, Nat. Cell Biol. 4:160-164). Furthermore, as this modification is absent in recombinant α-syn pffs (FIG. 29B, first lane on left, Luk et al., 2009, Proc. Natl. Acad. Sci. USA 106:20051-20056), the accumulation of phosphorylated α-syn (p-α-syn) reflects an intracellular modification. PBS-treated neurons did not show staining with 81A, a mAB specific for pSer129 (FIG. 29C, Waxman and Giasson, 2008, J Neuropathol. Exp. Neurol 67:402-416). However, neurons treated with α-syn-hWT pffs showed intense 81A immunostaining that was Tx-100 insoluble (FIG. 29C). Pff-induced aggregates exhibited morphologies ranging from small puncta to LN-like inclusions of variable lengths within neurites (FIGS. 29C, 29D, 30, and 32-35). Within neuronal perikarya, these α-syn accumulations resembled LBs observed in human PD brains (FIG. 29C inset). The α-syn-hWT pff-induced aggregates also occurred in primary cultures of cortical and midbrain dopaminergic neurons (FIG. 37A). Furthermore, neurons generated from other strains of mice as well as rats developed LB- and LN-like inclusions when treated with α-syn-hWT pffs, demonstrating that induction of α-syn pathology is a general feature of primary rodent neurons. P-α-syn positive aggregates (as detected by 81A) did not form in astrocytes (FIG. 37B). Moreover, the appearance of α-syn pathology required the presence of endogenous α-syn since α-syn-hWT pffs did not induce any pathology in primary neurons from α-syn −/− mice (FIG. 29C). Furthermore, monomeric α-syn did not induce α-syn inclusions, demonstrating that α-syn pffs alone seed the aggregates.

Immunoblot analyses were conducted on neuron lysates sequentially extracted with 1% Tx-100, followed by 2% SDS (FIG. 29B). In contrast to PBS-treated neurons, those treated with α-syn-hWT pffs for 14 days showed >80% reduction of α-syn in the Tx-100-soluble fraction accompanied by a concomitant appearance of α-syn in the SDS-extractable fraction. Immunoblots of the SDS-extractable fraction also showed insoluble p-α-syn. A mouse specific anti-α-syn antibody did not detect α-syn-hWT pffs (FIG. 29B, first lane on left), but detected bands in the neuron lysates similar to those labeled by the C terminus specific α-syn antibody and mAB 81A. In addition, higher-molecular weight species of α-syn were detected in the SDS fraction of all α-syn pffs-treated cultures, and likely correspond to oligomeric and/or ubiquitinated α-syn (Li et al., 2005, Proc. Natl. Acad. Sci. USA 102:2162-2167; Luk et al., 2009, Proc. Natl. Acad. Sci. USA 106:20051-20056; Sampathu et al., 2003, Am. J. Pathol. 163:91-100). Sequential extractions of primary hippocampal neurons from α-syn −/− mice 14 days after addition of α-syn-hWT pffs confirmed the absence of pathological α-syn or any other species of immunoreactive α-syn (FIG. 37C). Thus, these data demonstrate that α-syn pffs induced recruitment of soluble endogenous α-syn into insoluble, hyperphosphorylated α-syn aggregates.

Since α-syn is ubiquitinated in LBs and LNs, α-syn aggregates that formed 14 days after addition of α-syn-hWT pffs were studied, which showed that they were also ubiquitin positive (FIG. 29D), and colocalized with p-α-syn. Because the exogenous α-syn-hWT pffs are not ubiquitinated or phosphorylated (Luk et al., 2009, Proc. Natl. Acad. Sci. USA 106:20051-20056), these posttranslational modifications must occur intracellularly as endogenous mouse α-syn accumulates. Thus, these α-syn aggregates share hallmark features of PD-like LNs and LBs allowing us to conclude that misfolded α-syn pffs seed and recruit normal, endogenous α-syn to form pathologic aggregates.

Pffs from the NAC Domain of α-syn Are Sufficient to Seed Intracellular α-syn Aggregates

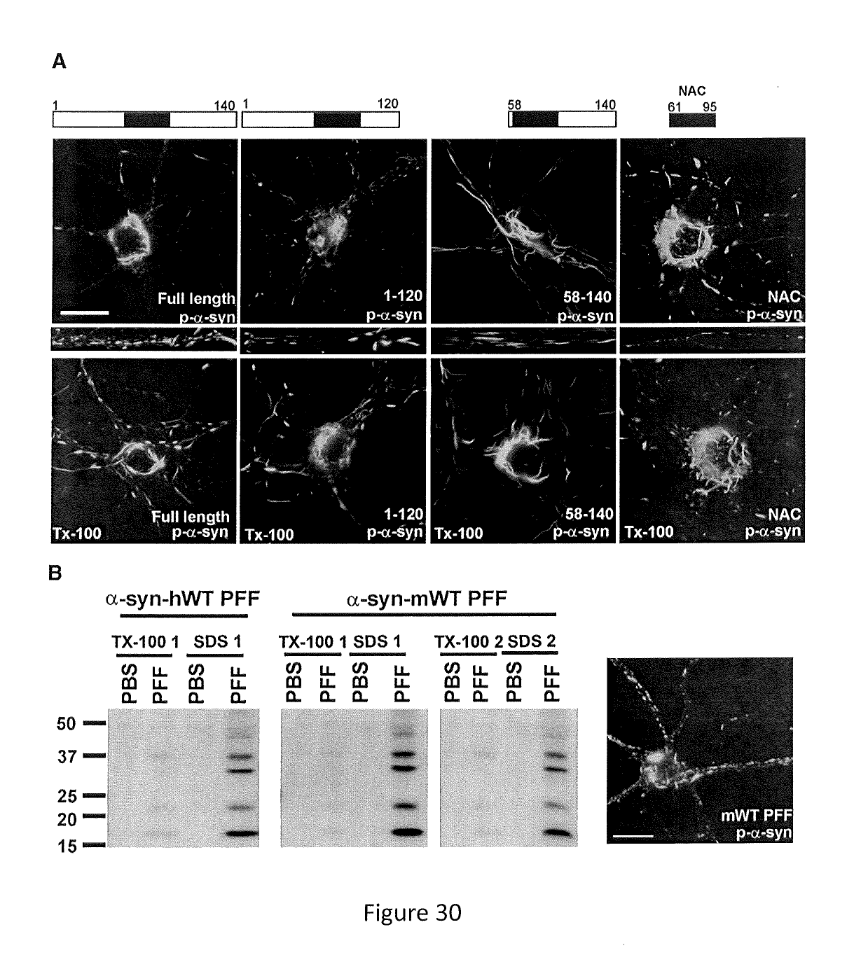

Previous in vitro studies have shown that recombinant α-syn protein lacking N- or C-terminal residues, or a synthetic peptide containing only the NAC domain (amino acid residues 61-95), assemble into α-syn amyloid fibrils, and nucleate full-length α-syn fibrillization (Giasson et al., 2001, J. Biol. Chem. 276:2380-2386; Han et al., 1995, Chem. Biol. 2:163-169; Kessler et al., 2003, Biochemistry 42:672-678; Luk et al., 2009, Proc. Natl. Acad. Sci. USA 106:20051-20056; Murray et al., 2003, Biochemistry 42:8530-8540; Serpell et al., 2000, Proc. Natl. Acad. Sci. USA 97:4897-4902). Thus, it was examined whether human α-syn pffs composed of α-syn-1-120, α-syn-1-89, α-syn-58-140, or α-syn-NAC could seed formation of LBs and LNs in neurons. It was observed that α-syn-1-120 and α-syn-1-89 pffs induced robust accumulation of endogenous p-α-syn aggregates that were Tx-100-insoluble (FIG. 30A), and they are morphologically indistinguishable from those formed by α-syn-hWT pffs. α-syn-58-140 pffs also seeded formation of endogenous mouse α-syn aggregates that were hyperphosphorylated (FIG. 30A). Moreover, pffs composed of only the central hydrophobic α-syn-NAC domain also resulted in endogenous mouse α-syn fibrillar LB-like aggregates that were Tx-100-insoluble. Overall, data presented herein demonstrate that α-syn pffs containing only the central, hydrophobic portion of α-syn-hWT are sufficient to seed conversion of endogenous α-syn into pathological aggregates.

Mice typically do not develop LBs except in the case of transgenic lines overexpressing mutant human α-syn. It was thus examined whether the formation of LB-like aggregates required human α-syn or whether they can be seeded by α-syn pffs generated from recombinant mouse WT α-syn (α-syn-mWT) (Touchman et al., 2001, Genome Res. 11:78-86). Immunoblots demonstrated that 14 days treatment of primary neurons with α-syn-mWT pffs induced appearance of p-α-syn in the Tx-100-insoluble fraction (FIG. 30B). Immunofluorescence also showed that α-syn-mWT pffs induced formation of p-α-syn aggregates in neurites and somata. Thus, pathological PD-like α-syn aggregates can be induced by α-syn-mWT pffs and does not require the human protein.

### Ultrastructure Analysis of α-syn Aggregates

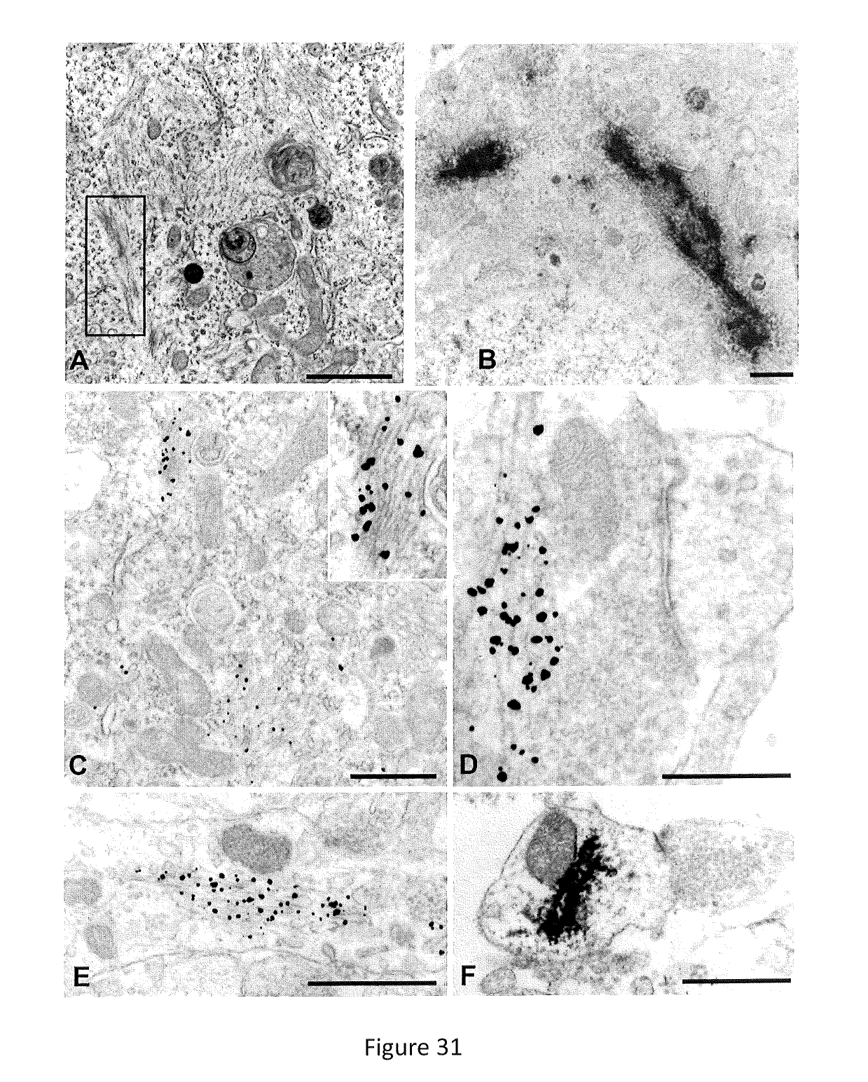

Examination of the α-syn aggregates using transmission and immuno-EM demonstrated abundant filaments in neurons treated with either α-syn-hWT or α-syn-1-120 pffs (FIG. 31A) for 14 days, but not PBS-treated neurons. Remarkably, inclusions composed of 14- to 16-nm-thick filaments were seen throughout the cytoplasm, visualized by transmission EM. Two different immuno-EM detection systems, horseradish peroxidase (HRP) and immunogold amplification, were used to demonstrate that fibrils composed of p-α-syn are found throughout the neuron. P-α-syn-positive fibrils were seen in the soma (FIGS. 31B and 31C), adjacent to the active zone of presynaptic terminals (FIG. 31D) and the postsynaptic terminal (FIG. 31F) and throughout processes (FIG. 31E). These data establish that the seeding and recruitment of endogenous mouse α-syn into hyperphosphorylated insoluble, filamentous aggregates recapitulate features of LBs and LNs in PD and other human synucleinopathies.

### Time and Concentration Dependence of α-syn Aggregate Formation

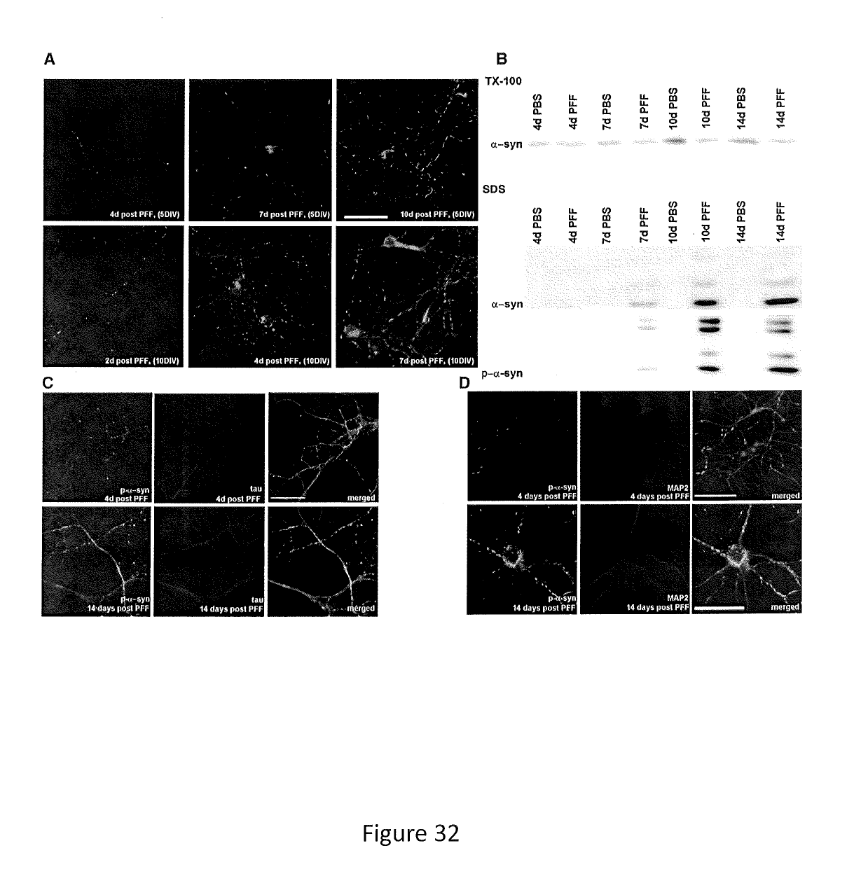

To determine the temporal sequence of α-syn aggregate formation, α-syn-hWT pffs were added to the neurons at DIV5. P-α-syn immunostaining was not detectable until 4 days later when small aggregates began to appear, exclusively in the neurites, albeit at low levels (FIG. 32A, upper series). By 7 days after α-syn-hWT pffs addition, there was an increase in α-syn pathology with some cell bodies showing α-syn accumulations. By 10 days post α-syn-hWT pff addition, the overall p-α-syn immunostaining was more intense, and p-α-syn aggregates in the neuritis appeared both punctate and fibrillar resembling LNs that were longer than the aggregates observed 4 or 7 days after α-synhWT pffs addition.

The sequence of events revealed by immunofluorescence was confirmed by biochemical experiments of sequentially extracted neurons (FIG. 32B). Four days after α-syn-hWT pffs addition, the majority of α-syn was found in the Tx-100-soluble fraction and showed levels similar to PBS-treated neurons. In PBS treated control neurons, there was an increase in α-syn levels by DIV10 as demonstrated previously (Murphy et al., 2000, J. Neurosci. 20:3214-3220). In contrast, 7-10 days after α-syn-hWT pff treatment, soluble levels of α-syn were reduced, accompanied by a concomitant increase of α-syn into the Tx-100-insoluble fraction. Thus, these data indicate that α-syn-hWT pff-induced recruitment of mouse α-syn into the insoluble fraction with a lag phase of a few days followed by a progressive increase in insoluble p-α-syn.

Since levels of α-syn and its concentration at the presynaptic terminals increase as primary neurons mature, (Murphy et al., 2000, J. Neurosci. 20:3214-3220; FIG. 32B, day 4 PBS versus day 10 PBS), it was examined whether adding pffs to mature neurons would enhance the rate of aggregation. When α-syn-hWT pffs were added to DIV10 neurons, aggregates were visible in neurites 2 days later (FIG. 32A, lower series), in contrast to 4 days required after addition of pffs to DIV5 neurons. By 4 days after α-syn-hWT pff treatment of DIV10 neurons, small punctate aggregates were detected throughout the neurites and some somata also showed accumulations, again unlike 4 days after adding pffs to DIV5 neurons in which α-syn pathology was exclusively in neurites. Seven days after α-syn-hWT pff treatment of DIV10 neurons, the pathology was extensive, similar to 10 days α-syn-hWT pff treatment of DIV5 neurons (FIG. 32A). Thus, α-syn aggregates develop faster in mature neurons, consistent with in vitro studies demonstrating that the rate of fibril formation positively correlates with α-syn concentrations (Wood et al., 1999, J. Biol. Chem. 274:19509-19512).

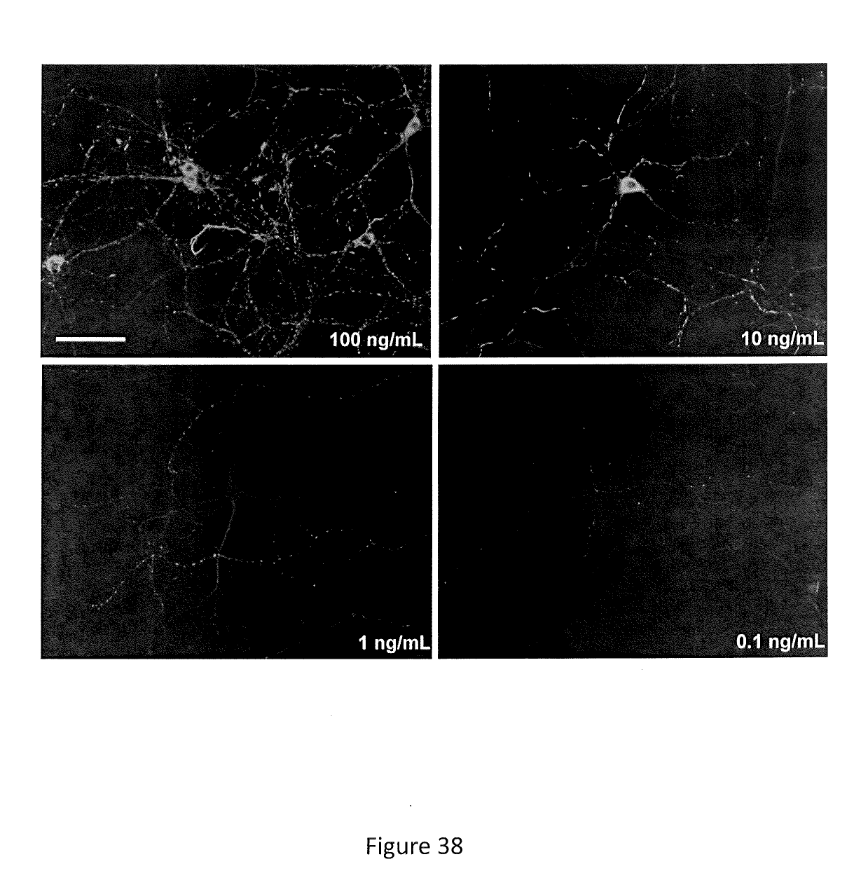

It was next examined whether the amount of α-syn pathology correlated with the amount of fibrils added. Progressive decreases in the amount of somatic and neuritic pathology correlated with 10-fold serial dilutions of α-syn-hWT pffs added (in ng/mL: 100, 10, 1, 0.1; FIG. 38). Thus, the rate and extent of pathology depends on the amount of α-syn pffs, and that small quantities of α-syn pffs are sufficient to seed α-syn aggregate formation, consistent with in vitro studies showing that the rate of seeded assembly depends on the initial concentrations of α-syn pffs (Wood et al., 1999, J. Biol. Chem. 274:19509-19512).

Initial Formation of α-syn Pathology within Axons

Because α-syn normally localizes to the presynaptic terminal and since α-syn puncta initially appeared in neurites, it was examined whether α-syn-hWT pffs recruited presynaptic α-syn into insoluble aggregates that then propagate from the axons to the cell bodies. To demonstrate that the pathology initiated in axons, double labeling immunofluorescence studies were conducted using a mAB specific for mouse tau (T49, an axonal marker) and 81A. P-α-syn aggregates colocalized predominately with tau 4 days after pff addition (FIG. 32C; upper panel), but not with the dendritic marker, microtubule associated protein 2 (MAP2) (FIG. 32D, upper panel), indicating that α-syn accumulations were initiated in axons. However, by 14 days, when more accumulations appeared in the somata, the α-syn aggregates were seen in axons (FIG. 32C, lower panel), in cell bodies, and proximal dendrites where they colocalized with MAP2 (FIG. 32D, lower panel). Thus, α-syn is recruited away from the presynaptic terminal with subsequent spread via axons to other parts of the polarized neuron.

### Enhanced Endocytosis of α-syn Pffs Increases the Extent of Pathology

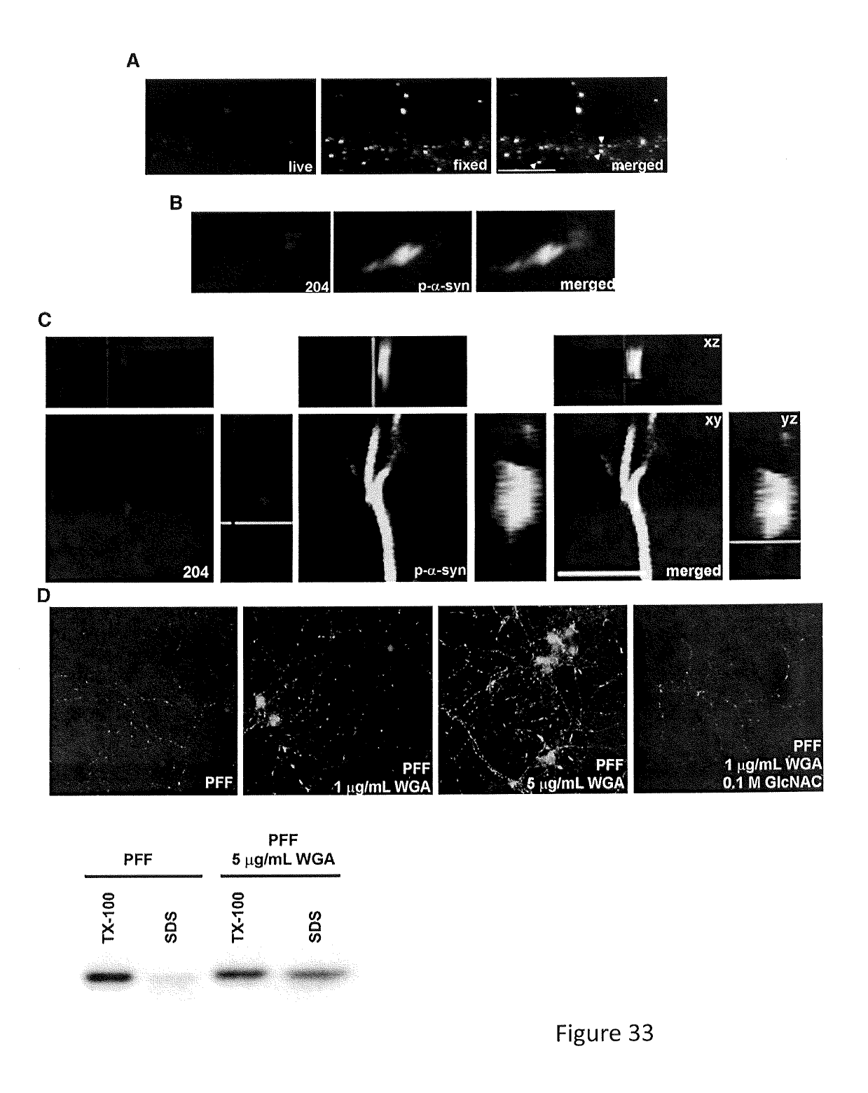

To determine whether α-syn-hWT pffs can gain access to the cytoplasm to seed recruitment of endogenous α-syn, two-stage immunofluorescence was performed using antibodies that recognize only human α-syn pffs. Live neurons were labeled at 4° C. with mAB Syn204 followed by fixation, permeabilization, and incubation with the antibody, LB509 (Giasson et al., 2000, J. Neurosci. Res. 59:528-533). Thus, mAB Syn204 labeled only extracellular hWT pffs whereas LB509 recognized both extracellular and intracellular hWT pffs. Many α-syn-hWT pffs remained outside the neuron and were double-labeled with both mAB Syn204 and LB509 (yellow in the merged image, FIG. 33A). However, significant amounts of small puncta labeled exclusively with LB509 (green, arrowheads highlight examples in the merged image), suggesting that α-synhWT pffs gain entry inside the neuron, as demonstrated previously for both α-syn and tau amyloid fibrils (Luk et al., 2009, Proc. Natl. Acad. Sci. USA 106:20051-20056; Guo and Lee, 2011, J. Biol. Chem. 286:15317-15331). Furthermore, double-labeling immunofluorescence in fixed, permeabilized neurons with mAB 81A and mAB Syn204 showed p-α-syn accumulating near seeds of α-syn-hWT pffs (FIG. 33B). A 3D view constructed from serial confocal images demonstrated colocalization between α-synhWT pffs (Syn204) and p-α-syn (81A) in the XY, XZ, and YZ planes (FIG. 33C), further confirming that intracellular pffs seed recruitment of endogenous α-syn. Since p-α-syn is exclusively intracellular, data presented herein indicate that pffs enter the cytoplasm where they initiate accumulation of pathologic p-α-syn.

To begin assessing the mechanism by which pffs gain entry to the cytoplasm, neurons were treated with α-syn-hWT pffs in the presence of wheat germ agglutinin (WGA) which binds N-acetylglucosamine (GlcNAC) and sialic acids at the cell surface and induces adsorptive-mediated endocytosis (Banks et al., 1998, J. Cell Sci. 111:533-540; Broadwell et al., 1988, Proc. Natl. Acad. Sci. USA 85:632-636; Gonatas and Avrameas, 1973, J. Cell Biol. 59:436-443). To determine the effects of WGA on formation of α-syn aggregates, neurons were treated at DIV5 and fixed for immunofluorescence 4 days later. When incubated with α-syn-hWT pffs alone, few p-α-syn puncta were visible in a subset of neurites (FIG. 33D). Co-incubation of pffs with WGA dose-dependently increased the extent of p-α-syn pathology. In addition to small puncta, longer, continuous p-α-syn filaments were visible, and α-syn pathology was present in the cell body, particularly with 5 μg/mL of WGA treatment. Furthermore, the addition of 0.1 M GlcNAc, a competitive inhibitor of WGA, reduced the effects of WGA on α-syn pff-induced aggregate formation. Immunoblots of sequentially extracted neurons confirm that WGA-mediated endocytosis enhances formation of pathologic α-syn. Four days after treatment with α-syn-hWT pffs alone, the majority of α-syn remained in the Tx-100 extractable fraction, whereas coincubation of α-syn-hWT pffs with 5 g/mL of WGA increased the amount of Tx-100 insoluble α-syn. Taken together, the data presented herein indicates that α-syn pffs gain access to the neuronal cytoplasm by adsorptive endocytosis.

### Intracellular Propagation of Pathologic α-syn

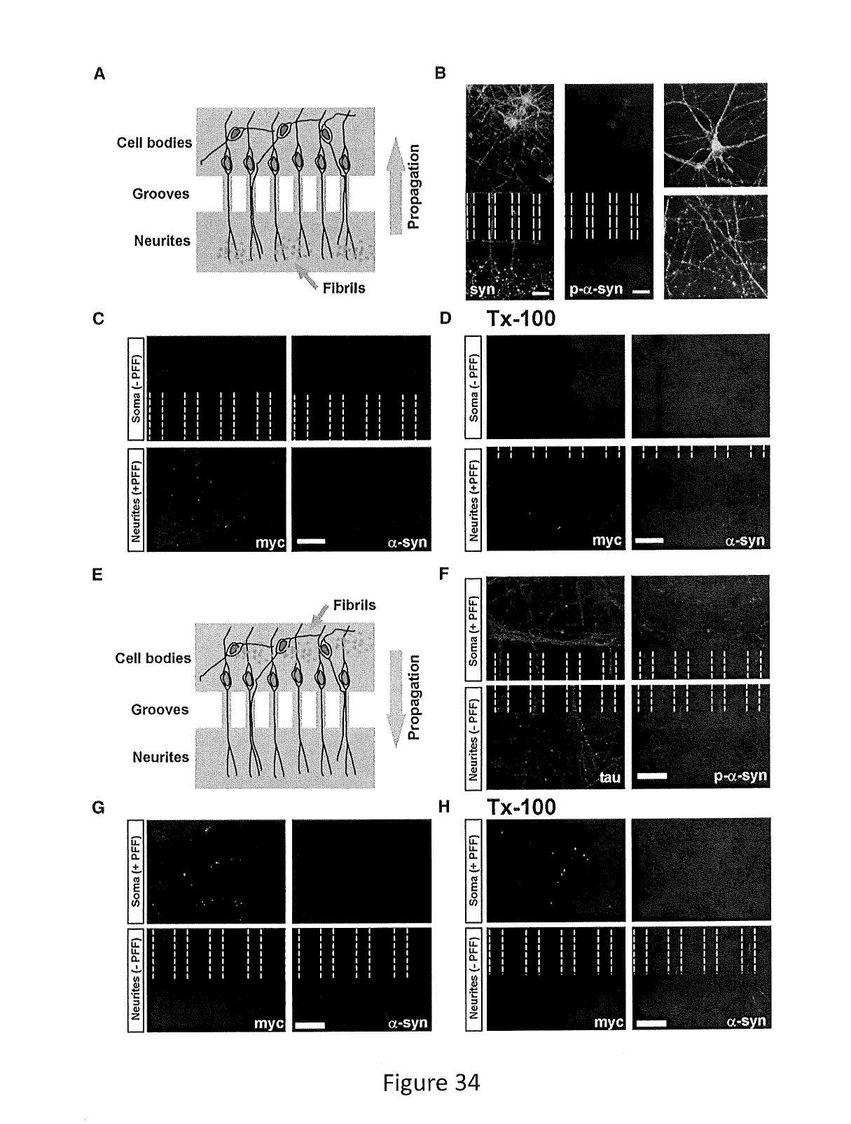

To determine whether direct addition of α-syn pffs to either neurites or somata leads to propagation of pathologic α-syn aggregates throughout the neuron, microfluidic culture devices that isolate the neuronal processes from the cell bodies via a series of interconnected microgrooves were utilized (Taylor et al., 2005, Nat. Methods 2:599-605). C-terminally myc-tagged α-syn-1-120 pffs added to the neuritic chamber (FIG. 34A) resulted in p-α-syn-positive aggregates within axons and cell bodies (FIGS. 34B and 34C). Aggregates were morphologically identical to those seen in primary neurons directly exposed to pffs, and they were also insoluble in Tx-100 (FIG. 34D). Anti-myc immunostaining suggested that pffs did not enter into the somal compartment (FIGS. 34C and 34D) or microgrooves. Thus, these data indicate that pathological p-α-syn can form within isolated neurites and is propagated retrogradely to the cell bodies.

Neuronal somata that were isolated from neurites in the microfluidic devices were also exposed to α-syn-1-120-myc pffs and the extent of α-syn pathology in the processes was assessed (FIG. 34E). As expected, neurons treated with α-syn-1-120-myc pffs formed somatic p-α-syn pathology (FIG. 34F). P-α-syn aggregates were also detected in axons that extended through the microgrooves into the neurite chamber, as revealed by colabeling with tau (FIG. 34F). Again, α-syn aggregates throughout the axon were Tx-100-insoluble, and immunofluorescence using the anti-myc antibody demonstrated that α-syn-1-120-myc pffs were confined to the somatic compartment (FIGS. 34G and 34H). Thus, pathologic p-α-syn aggregates also propagate in the anterograde direction.

### Formation of Aggregates Leads to Neuron Loss and Diminished Levels of Select Synaptic Proteins

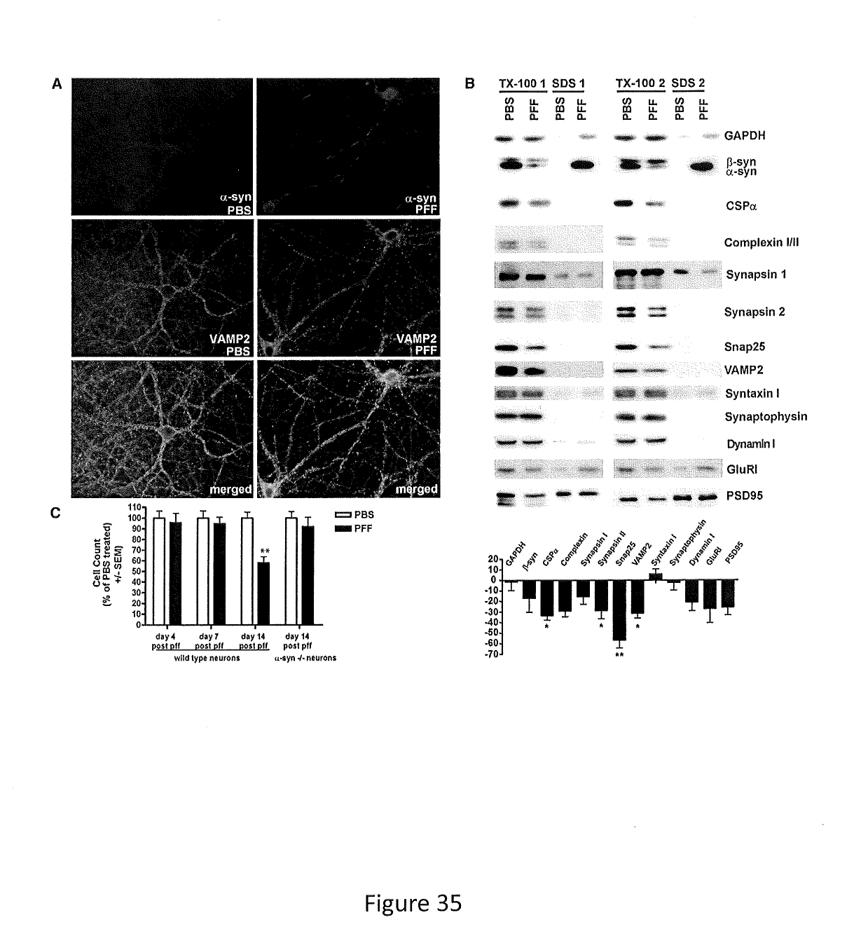

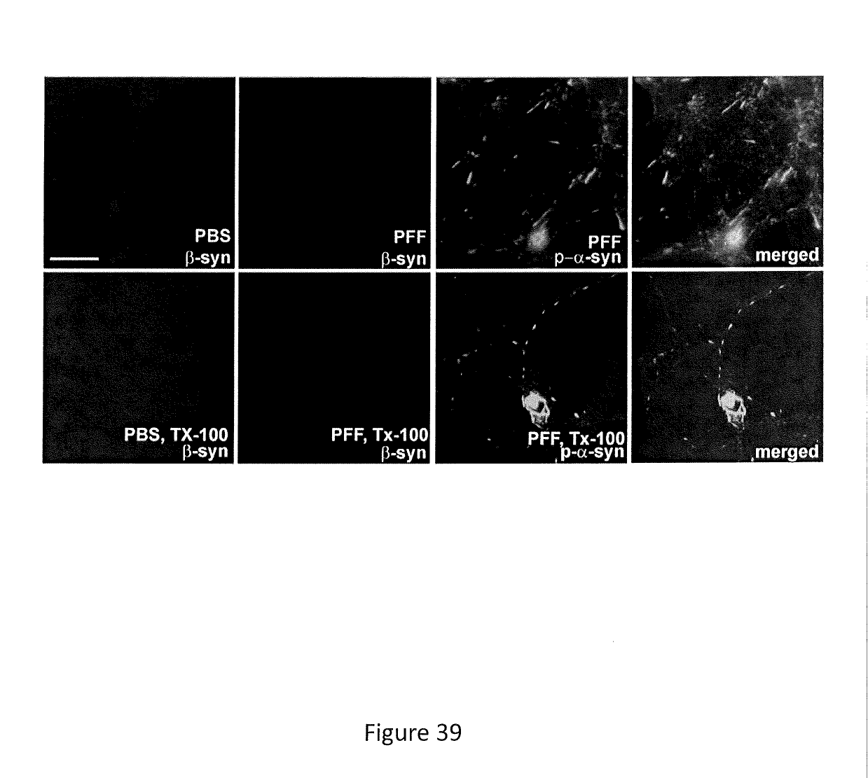

α-syn resides predominantly at the presynaptic terminal and previous reports indicate that it acts as a co-chaperone, in concert with another chaperone, cysteine-string protein a (CSPα), to maintain SNARE complex formation by binding to VAMP2/synaptobrevin 2 (Burre et al., 2010, Science 329:1663-1667; Chandra et al., 2005, Cell 123:383-396; Greten-Harrison et al., 2010, Proc. Natl. Acad. Sci. USA 107:19573-19578). Thus, the consequences of recruitment of α-syn into insoluble aggregates on synaptic protein distribution and expression were examined. In PBS-treated control neurons, α-syn colocalized with VAMP2 at the presynaptic terminal. Addition of α-syn-hWT pffs led to a depletion of α-syn from the presynaptic terminal such that it showed minimal colocalization with presynaptic VAMP2 (FIG. 35A). To further investigate the molecular consequences of recruitment of endogenous α-syn into insoluble aggregates, additional synaptic proteins that could be impacted by the pathological sequestration of α-syn into aggregates and away from the presynaptic terminal were examined. Although β-synuclein (β-syn), another member of the same family of neuronal proteins as α-syn, but lacking the NAC domain, colocalized with α-syn at presynaptic terminals in control neurons (Murphy et al., 2000, J. Neurosci. 20:3214-3220), α-syn-hWT pff addition did not change the presynaptic localization of β-syn (FIG. 39). Furthermore, Tx-100 extraction showed that, unlike pathological α-syn, which localized to detergent insoluble aggregates, β-syn remained soluble (FIG. 39). Immunoblot analyses showed that endogenous β-syn was Tx-100 soluble 14 days after adding α-syn pffs (FIG. 35B) and protein levels in pff-treated neurons were not statistically significantly different from PBS-treated neurons. Thus, like LBs in PD brains, the aggregates that developed in primary neurons are composed of insoluble α-syn, but not β-syn (Spillantini et al., 1998, Neurosci. Lett. 251:205-208). Importantly, this is consistent with the selective recruitment of α-syn by pffs as opposed to the indiscriminate disruption of adjacent presynaptic components.

Nonetheless, statistically significant reductions were able to be detected in a subpopulation of synaptic proteins two weeks after the addition of α-syn-hWT pffs, including the synaptic vesicle-associated SNARE proteins, Snap25 and VAMP2, as well as soluble proteins that participate in SNARE complex assembly or the exo-endocytic synaptic vesicle cycle such as CSPα, and synapsin II (FIG. 35B). Levels of other synaptic proteins showed slight, but not statistically significant reductions. Changes were not observed in GAPDH, the plasma membrane-associated SNARE protein, syntaxin 1, or the transmembrane synaptic protein synaptophysin.

Since loss of synaptic proteins may correlate with neurodegeneration, it was examined whether the accumulation of α-syn aggregates leads to neuron loss. NeuN-positive neurons were counted in cultures treated with PBS or α-syn-hWT pffs 4, 7, or 14 days after α-syn pff addition. While there was a slight but not statistically significant decrease in number of neurons 7 days after α-syn-hWT pff treatment, by 14 days after pff treatment, there was a significant 40% decrease in neurons relative to PBS controls (FIG. 34C). Cell death did not occur in α-syn-hWT pff-treated neurons derived from α-syn −/− mice, demonstrating that intracellular aggregates, rather than the mere addition of exogenous pffs, caused neuron death. Finally, using ethidium homodimer to detect dead cells and Hoechst 33342 to detect total cells, we demonstrated a ˜68% increase in cell death 14 days after pff-treatment (56.6%) versus PBS-treated (33.6%) neurons.

### Disruption in Network Activity Matches the Progression of α-syn Pathology

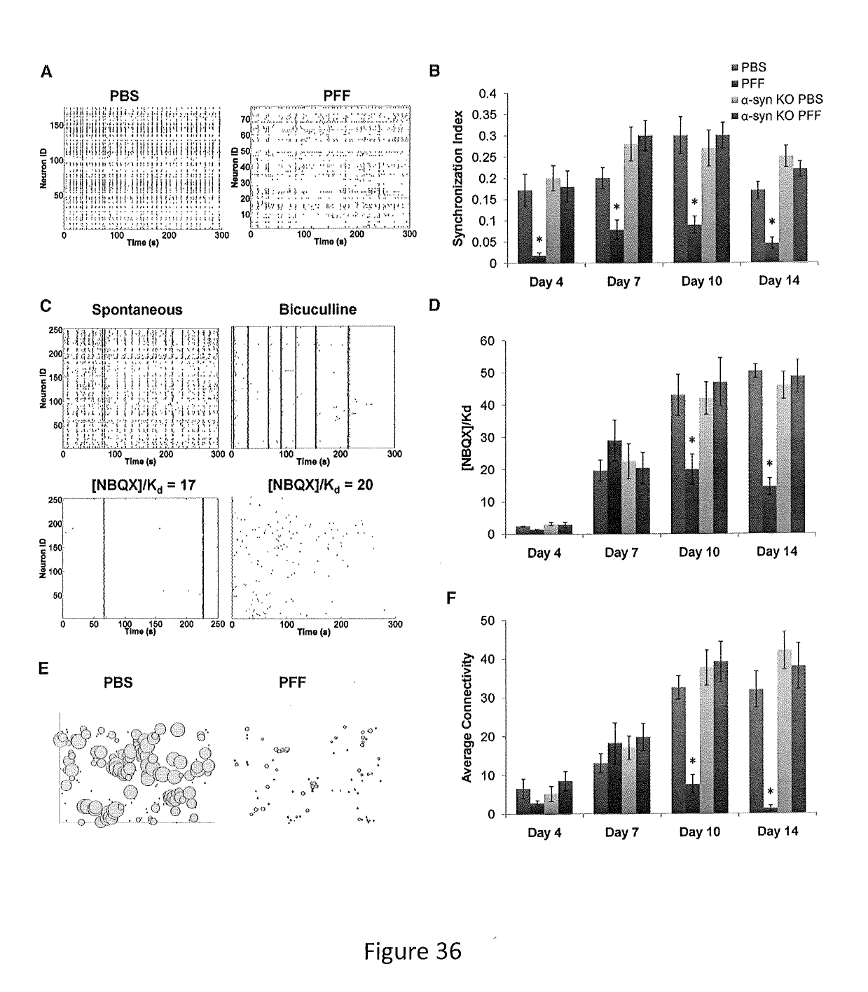

The decreased levels of synaptic proteins suggest impairment in neural network activity following accumulation of α-syn inclusions. Calcium imaging of hippocampal neurons loaded with the calcium-sensitive fluorescent dye, Fluo-4 AM, was performed to investigate the effect of α-syn aggregates on the activity patterns of the in vitro neural network established by these cultured neurons. The spontaneous activity of neurons treated with PBS was characterized by flickering events, intermixed with network-wide bursts when nearly all the neurons were simultaneously firing as reflected by a high synchronization index (FIG. 36B). In contrast, neurons treated with α-syn-hWT pffs showed a significant decrease in synchronized activity as early as 4 days after treatment. At this time point, low levels of α-syn aggregates were visualized exclusively in axons by immunofluorescence microscopy, and no pathological α-syn was detected biochemically (FIGS. 32A and 32B). Yet, this was sufficient to impair coordinated network activity. This reduction in synchronized activity persisted at 7, 10, and 14 days after α-syn-hWT pff treatment (FIG. 36B). In contrast, α-syn-hWT pff-treated neurons from α-syn −/− mice showed no impairments in the synchronization index, indicating that these effects are selective for neurons harboring α-syn aggregates and do not result from exogenously added pffs. It was next determined whether the progressive recruitment of α-syn into pathologic aggregates correlated with changes in the excitatory tone of the network. First, synchronous oscillations were forced using the GABA(A) antagonist, bicuculline, to abolish inhibitory input, followed by increasing doses of the AMPA receptor antagonist, NBQX, until synchronous oscillations stopped (FIG. 36C). The final concentration of NBQX required to impair activity within the excitatory network determined the excitatory tone. No significant changes in excitatory tone was detected in cultures 4 or 7 days after α-syn-hWT pff treatment but by 10 and 14 days after treatment, when increasing accumulation of neuritic and perikaryal pathology was observed, there were significant reductions in excitatory tone (FIG. 36D), reflecting compromised synaptic activity. Again, neurons from α-syn −/− mice did not show impairments in excitatory tone at 10 and 14 days after pff treatment, confirming that the effects result from the accumulation of endogenous α-syn aggregates.

Since spatiotemporal patterns of activity are shaped by the underlying connectivity architecture and the relative balance of excitation and inhibition, network activity patterns were used to determine the functional connectivity in PBS and α-synhWT pff-treated neurons. As neurons matured in vitro, the number of functional connections increased and eventually plateaued (FIG. 36F). The timeframe for this correlated well with neurite sprouting and synapse stabilization based on previous studies of developing connections in vitro (Soriano et al., 2008, Proc. Natl. Acad. Sci. USA 105:13758-13763). However, in α-syn-hWT pff-treated WT, but not α-syn −/− neurons, the maturation of functional connections never reached the level achieved in PBS-treated cultures, as a significant reduction was observed 10 days after α-syn-hWT pff treatment (FIG. 36F). This functional connectivity was severely compromised 14 days after treatment and the network consisted of just a few sparse connections at this time point (FIGS. 36E and 36F). In summary, the formation of insoluble aggregates of endogenous α-syn results in early disruption in coordinated network activity. Later, as more α-syn inclusions develop and propagate throughout the neuron, excitatory tone is decreased and functional connectivity is greatly reduced.

### A Neuronal Culture Model of PD-like α-syn Inclusion Formation

As demonstrated herein seeds derived from α-syn amyloid fibrils generated with pure recombinant full-length and truncated human WT α-syn, when directly added to mouse primary hippocampal neurons, are internalized and induce the recruitment of endogenous soluble α-syn into insoluble pathologic LB-like and LN-like α-syn aggregates resembling those found in human synucleinopathies. Indeed the verisimilitude of these α-syn aggregates in cultured mouse neurons to LBs and LNs found in PD brains is striking because they are found in perikarya and extensively in neurites, insoluble, filamentous by EM and immuno-EM, hyperphosphorylated, and ubiquitinated, and they exclude β-syn (Baba et al., 1998, Am. J. Pathol. 152:879-884; Duda et al., 2000, J. Neuropathol. Exp. Neurol. 59:830-841; Fujiwara et al., 2002, Nat. Cell Biol. 4:160-164; Spillantini et al., 1998, Neurosci. Lett. 251:205-208). Notably, these aggregates initially formed within axons, sequestering endogenous α-syn away from presynaptic terminals, followed by propagation into the somata. Over time, formation of these α-syn aggregates leads to selective alterations in synaptic proteins, compromises neuronal excitability and connectivity, and culminates in neuron death. Thus, as described herein, a neuronal culture model of PD-like α-syn inclusion formation has been developed, which allows for the dissection of the mechanisms leading to the formation of LBs and LNs, as well as for studies of the impact of these inclusions on the function and viability of affected neurons. Moreover, since the majority of PD and DLB cases are sporadic and are not caused by mutations or overexpression of α-syn, this neuronal model system provides a means to study the pathogenesis of α-syn in sporadic PD, as well as other α-synucleinopathies.

As described herein, α-syn pffs made from pure, recombinant protein are highly potent in the recruitment of the endogenously expressed protein into LB-like and LN-like α-syn pathology, in contrast to previous studies that have relied on experimental manipulations such as protein overexpression of WT and mutant proteins, and/or extrinsic factors to introduce pffs into cells (Clavaguera et al., 2009, Nat. Cell Biol. 11:909-913; Frost et al., 2009, J. Biol. Chem. 284:12845-12852; Guo and Lee, 2011, J. Biol. Chem. 286:15317-15331; Luk et al., 2009, Proc. Natl. Acad. Sci. USA 106:20051-20056). These results thus provide support that α-syn amyloid fibrils alone are sufficient to seed and drive α-syn pathology in healthy neurons. Indeed, the presently described findings can plausibly account for the observation that fetal grafts of embryonic neurons in diseased PD brains develop LBs over time, since this could be caused by the direct uptake of fibrillar α-syn seeds from diseased neurons in the brains of these patients (Li et al., 2008, Nat. Med. 14:501-503; Kordower et al., 2008a, Nat. Med. 14:504-506; Kordower et al., 2008b, Mov. Disord. 23:2303-2306). While not being held to any particular theory, it is believed that the data presented herein suggest a pathological mechanism whereby misfolded α-syn species can amplify and propagate in the CNS. The results presented herein, provides evidence that small amounts of misfolded α-syn pffs can trigger the spread of α-syn pathology throughout the entire neuron.

Two-stage immunofluorescence was used to distinguish extracellular from internal pffs. Confocal microscopy was used to demonstrate colocalization between pffs and p-α-syn suggest that small amounts of α-syn pffs gain access to the neuronal cytoplasm where they can seed α-syn misfolding and accumulation into hyperphosphorylated α-syn inclusions. Coincubation of α-syn pffs with WGA enhances the extent of pathology, implicating adsorptive-mediated endocytosis as a potential mechanism by which pffs gain entry to the neuron. Without wishing to be bound by any particular theory, it is believed that α-syn pffs are internalized and released into the cytosol and thereby efficiently induce pathology. High concentrations of α-syn are present in presynaptic terminals, where it associates with vesicular membranes and undergoes rapid exchange between bound and unbound states (Fortin et al., 2010, Mov. Disord. 25(Suppl 1):S21-S26). Thus, high local concentrations of presynaptic α-syn, coupled with its dynamic characteristics, may facilitate recruitment of endogenous mouse α-syn by the internalized α-syn pffs to form insoluble fibrils.

As described elsewhere herein, formation of α-syn pathology is more efficient in mature neurons with higher levels of α-syn expression at presynaptic terminals. Interestingly, levels of α-syn increase with age (Chu and Kordower, 2007, Neurobiol. Dis. 25:134-149) and α-syn gene duplication and triplication can lead to PD (Singleton et al., 2003, Science 302(5646):841). The data presented herein demonstrates that normal neuronal expression of α-syn is sufficient for seeding of α-syn pathology after exposing these neurons to α-syn pffs.

The presently described model has allowed the determination of some of the consequences of potential α-syn dysfunction resulting from its recruitment into inclusions, which include reduced levels of synaptic proteins, impaired neuronal function and eventual death of affected neurons. Diminished neuronal synchronization begins early after pff addition when small aggregates are visible only in axons, suggesting that even a minor burden of α-syn pathology can have a major impact on the coordinated activation of neuronal ensembles. By 10 days and 14 days after pff treatment, when pathology is extensive, neuronal excitability and connectivity is substantially reduced, which may be accounted for by the reductions in presynaptic proteins. Alterations in the expression and localization of these proteins occurs upon ablation of all three synuclein family members or overexpression of α-syn (Burre et al., 2010, Science 329:1663-1667; Greten-Harrison et al., 2010, Proc. Natl. Acad. Sci. USA 107:19573-19578; Nemani et al., 2010, Neuron 65:66-79). Sequestration of α-syn away from the presynaptic terminal into insoluble inclusions may impair the homeostasis of presynaptic proteins and consequently, synaptic vesicle exocytosis, as suggested by previous studies demonstrating that α-syn, in cooperation with CSPα, may act as a chaperone to maintain presynaptic SNARE complex assembly (Burre et al., 2010, Science 329:1663-1667; Chandra et al., 2005, Cell 123:383-396). Over time, disruptions in the synaptic vesicle exo-endocytic cycle may contribute to neurodegeneration found in PD and DLB.

For enigmatic reasons, LB pathology in sporadic PD disease progresses in a temporally and topologically sequential manner. Data presented herein suggest that LB/LN pathology can be induced by misfolded α-syn and is propagated within neurons. As shown herein, small amounts of α-syn pffs directly induce endogenous α-syn to form pathological aggregates that are spread throughout the neuron and accumulate as LB-like and LN-like inclusions support this unifying mechanism of disease progression and therefore have important implications for understanding the onset and progression as well as etiopathogenesis of sporadic PD and other neurodegenerative disorders. The results presented herein opens up new avenues of research into understanding mechanisms underlying the development LB and LN pathology, their impact on neuronal function, and discovering therapies for PD and other α-synucleinopathies.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A cell culture model for alpha synuclein inclusion formation, said model comprising a cell population and exogenous alpha synuclein fibrils.

2. The cell culture model of claim 1, wherein said cell population comprises a neuronal cell.

3. The cell culture model of claim 1, wherein said cell population comprises a non-genetically modified cell.

4. The cell culture model of claim 1, wherein said cell population comprises a cell engineered to express a nucleic acid encoding alpha synuclein.

5. The cell culture model of claim 4, wherein said cell further comprises a nucleic acid encoding a detectable protein.

6. The cell culture model of claim 1, comprising a culture medium comprising exogenous alpha synuclein fibrils.

7. The cell culture model of claim 1, further comprising wheat germ agglutinin (WGA).

8. The cell culture model of claim 1, wherein said exogenous alpha synuclein fibrils are derived from a mammalian alpha synuclein.

9. The cell culture model of claim 8, wherein said mammalian alpha synuclein is selected from the group consisting of mouse, rat, primate, and human.

10. The cell culture model of claim 1, wherein said exogenous alpha synuclein fibrils is derived from a human alpha synuclein or a fragment thereof selected from the group consisting of full-length alpha synuclein (α-syn), α-syn-1-120, α-syn-1-89, α-syn-58-140, α-syn-61-95, and any combination thereof.

11. A cell culture model for tau inclusion formation, said model comprising a cell engineered to express a nucleic acid encoding tau.

12. The cell culture model of claim 11, wherein said tau has a P301L mutation.

13. The cell culture model of claim 11, wherein said cell is a neuronal cell.

14. The cell culture model of claim 11, wherein said cell further comprises a nucleic acid encoding a detectable protein.

15. The cell culture model of claim 11, comprising a culture medium comprising exogenous tau.

16. A method of identifying a test agent that inhibits filament aggregation, said method comprising contacting a cell culture model for filament aggregation with said test agent and comparing the amount of aggregation in cells in said cell culture model with the amount of aggregation in cells in an otherwise identical cell culture model not contacted with said test agent, wherein a lower level of aggregation in the presence of said test agent identifies the test agent as an inhibitor of filament aggregation.

17. The method of claim 16, wherein said cell culture model is a model for alpha synuclein inclusion formation, wherein said model comprises a cell population and exogenous alpha synuclein fibrils.

18. The method of claim 16, wherein said cell culture model is a model for tau inclusion formation comprising a cell engineered to express a nucleic acid encoding tau.

19. A method of identifying a gene product that modulates filament aggregation in a cell, the method comprising contacting a cell culture model for filament aggregation with a modulator of gene expression and comparing the amount of aggregation in cells in said cell culture model with the amount of aggregation in cells in an otherwise identical cell culture model not contacted with said modulator of gene expression, wherein a change in the level of aggregation in the presence of said modulator of gene expression identifies the modulator of gene expression as a gene product that modulates filament aggregation.

20. The method of claim 19, wherein said cell culture model is a model for alpha synuclein inclusion formation, wherein said model comprises a cell population and exogenous alpha synuclein fibrils.

21. The method of claim 19, wherein said cell culture model is a model for tau inclusion formation comprising a cell engineered to express a nucleic acid encoding tau.
